SEQUENCE LISTING (122 sequences)

SEQ ID NO: 1 - protein, 155 aa, Escherichia coli
MSQVILDLQLACEDNSGLPEESQFQTWLNAVIPQFQEESEVTIRVVDTAESHSLNLTYRGKDKPTNVLSFPFEVPPGMEMSLLGDLVICRQVVEKEAQEQGKPLEAHWAHMVVHGSLHLLGYDHIEDDEAEEMEALETEIMLALGYEDPYIAEKE

SEQ ID NO: 2 - protein, 155 aa, Shigella flexneri; identical to SEQ ID NO: 1

SEQ ID NO: 3 - protein, 155 aa, Shigella sonnei; identical to SEQ ID NO: 1

SEQ ID NO: 4 - protein, 155 aa, Shigella boydii; identical to SEQ ID NO: 1

SEQ ID NO: 5 - protein, 155 aa, Shigella dysenteriae
MSQVILDLQLACEDNSGLPEESQFQTWLNAVIPQFQEESEVTVRVVDTAESHSLNLTYRGKDKPTNVLSFPFEVPPGMEMSLLGDLVICRQVVEKEAQEQGKPLEAHWAHMVVHGSLHLLGYDHIEDDEAEEMEALETEIMLALGYEDPYIAEKE

SEQ ID NO: 6 - protein, 155 aa, Escherichia coli
MSQVILDLQLACEDNSGLPEESQFQTWLNAVIPQFQEESEVTIRVVDTAESHSLNLTYRGKDKPTNVLSFPFEVPPGMEMSLLGDLVICRQMVEKEAQEQGKPLEAHWAHMVVHGSLHLLGYDHIEDDEAEEMEALETEIMLALGYEDPYIAEKE

SEQ ID NO: 7 - protein, 155 aa, Escherichia albertii
MSQVILDLQLACEDNSGQPEESQFQTWLNAVIPQFQEESEVTIRLVDTAESHSLNLTYRGKDKPTNVLSFPFEVPPGMEMSLLGDLVICRQVVEQEALEQGKPLEAHWAHMVVHGSLHLLGYDHIEDDEAEEMEALETEIMLALGYEDPYIAEKE

SEQ ID NO: 8 - protein, 157 aa, Salmonella enterica
MSQVILDLQLACENHAGLPDEAQFQRWLDGVIPQFQEEAEVTIRLVDEAESHDLNLTYRGKDKPTNVLSFPFEAPPGIEMPLLGDLIICRQVVEQEAQEQSKPLEAHWAHMVVHGSLHLLGYDHIDDDEAEEMESLETEIMLAMGYEDPYIAEKIAE

SEQ ID NO: 9 - protein, 157 aa, Salmonella typhi; identical to SEQ ID NO: 8

SEQ ID NO: 10 - protein, 157 aa, Salmonella choleraesuis; identical to SEQ ID NO: 8

SEQ ID NO: 11 - protein, 157 aa, Salmonella paratyphi
MSQVILDLQLACENHAGLPDEAQFQRWLDGVIPQFQEEAEVTIRLVDEAESHDLNLTYRGKDKPTNVLSFPFEAPPGIEMPLLGDLIICRQVVEQEAQEQSKPLEAHWAHMVVHGSLHLLGYDHIDDDEAEEMESLETEIMLAMGYEDPYIAEKITE

SEQ ID NO: 12 - protein, 155 aa, Enterobacter sakazakii
MSQVILDLQVACEDTTGLPDEAQFQTWLNAVVPQFQEESEVTVRLVDEAESHDLNLTYRGMDKPTNVLSFPFEAPPGIDMPLLGDLIICRQVVEREAREQEKPLEAHWAHMVVHGSLHLLGYDHIEDDEAEEMEGIETEIMLALGFDDPYIAEKA

SEQ ID NO: 13 - protein, 157 aa, Salmonella typhimurium
MSQVILDLQLACENHAGLPDEAQFQRWLDGVIPQFQEEAEVTIRLVDEAESHDLNLTYRGKDKPTNVLSFPFEAPAGIEMPLLGDLIICRQVVEQEAQEQSKPLEAHWAHMVVHGSLHLLGYDHIDDDEAEEMESLETEIMLAMGYEDPYIAEKIAE

SEQ ID NO: 14 - protein, 157 aa, Klebsiella pneumoniae
MSQVILDLQLACEETSGLPDEALFQRWVDAVIPPFQEESELTIRLVDVAESHELNLTYRGKDKPTNVLSFPFEAPPGIEMPLLGDLIICRQVVEQEASEQGKPLEAHWAHMVVHGSLHLLGYDHIEDDEAEEMEGLETEIMLALGYEDPYISEKIAE

SEQ ID NO: 15 - protein, 155 aa, Citrobacter koseri
MSQVILDLQLACEDHSGLPEESQFQTWLNAVIPQFQEESEVTIRLVDEAESHDLNLTYRGKDKPTNVLSFPFEAPPGMEMPLLGDLIICRQVVEQEAKEQDKPLEAHWAHMVVHGSLHLLGYDHIEDDEAEEMEALETEIMLALGYEDPYISEKD

SEQ ID NO: 16 - protein, 155 aa, Enterobacter sp.
MSQVILDLQLACEDNSGLPEESQFQKWLDAVIPQFQEESEVTIRVVDEAESHELNLTYRGKDKPTNVLSFPFEAPPGIELPLLGDLIICRQVVEQEAKEQQKPLDAHWAHMVIHGSLHLLGYDHIEDEEAEEMESLETEIMLALGYEDPYIAEKE

SEQ ID NO: 17 - protein, 157 aa, Serratia proteamaculans
MSQVILDLQLACADNSGLPDEATFQRWLEGVLPQFQEEAEVTIRLVDEAESNELNLTYRGMDKPTNVLSFPFEAPPGIELPLLGDLIICRQVVEREAAEQDKALEAHWAHMVVHGSLHLLGYDHIEDDEAEEMESLETEIMHGLGYPDPYLAEKDPL

SEQ ID NO: 18 - protein, 155 aa, Erwinia tasmaniensis
MSGVILDLQLACENEQGLPAETDFQRWLEAVLPQFQPESEVTIRLVDEAESRELNHTYRSKDKPTNVLSFPFEAPPGIELPLLGDLIICRQVVEQEAVEQGKTREAHWAHMVIHGSLHLLGYDHIEDDEAEEMESLETEIMLALGYPDPYISEKE

SEQ ID NO: 19 - protein, 157 aa, Yersinia enterocolitica
MSQVILDLQIACADSQGLPTEADFQHWLEAVLPQFQEVSEVTIRVVDEAESHELNLTYRGKDKPTNVLSFPFEAPPEIELPLLGDLIICRQVVEQEAVEQEKALLAHWAHMVVHGSLHLLGYDHIVDDEAEEMESIETEIMQSLGYPDPYISEKDPE

SEQ ID NO: 20 - protein, 155 aa, Photorhabdus luminescens
MSSVILDLQIACEHSQGLPKETLFQHWLDGVLPQFQSESEVTIRIVDEAESHDLNLTYRGKNKPTNVLSFPFEAPPEIDLPLLGDLIICRQVVEKEAEEQQKTIEEHWAHMVVHGCLHLLGYDHIEDDEAEEMESLETEIMQKLGYADPYLAEKE

SEQ ID NO: 21 - protein, 157 aa, Yersinia intermedia
MSQVILDLQIACADSQGLPAEADFQRWLEAVLPQFQDVAEVTVRLVDEAESHELNLTYRGKDKPTNVLSFPFEAPPEIELPLLGDLIICRQVVEQEAIEQEKALLAHWAHMVVHGSLHLLGYDHIVDDEAEEMESIETEIMQSLGYPDPYISEKDPV

SEQ ID NO: 22 - protein, 157 aa, Yersinia bercovieri
MSQVILDLQIACANSQGLPTEADFQQWLEAVLPQFQEVSEVTIRLVDEAESHQLNLTYRGKDKSTNVLSFPFEAPPEIELPLLGDLIICRQVVEQEAVEQEKALFAHWAHMVVHGSLHLLGYDHIVDDEAEEMESIETEIMQSLGYPDPYISEKESD

SEQ ID NO: 23 - protein, 250 aa, Vibrio vulnificus
MSRRLTYLAVQNRAFVMPSKCSMKWKRSALTFSNPMTSFATQSSRASSMPTRSGKHKTKKSVKSLNNVAEKSAMPNCLKPQKPSFLHKSARIRKPHMAIELDLQLAVEDQNGLPSAQDFQTWLDKTIPPFQPQAEVTIRIVDSQESHQLNHDYRGKDKPTNVLSFPFEAPPGMEMDLLGDLVICRQVVEQEAIEQDKPLMAHWAHMVVHGSLHLLGYDHIEDDEAEEMESLETEIMQGMGFTDPYLAEKE

SEQ ID NO: 24 - protein, 155 aa, Providencia stuartii
MSEVILDLQLACEETTGLPDEALFQRWLEAVLPKFQPQSEVTIRIVDEEESHHLNLTYRGKDKPTNVLSFPFEAPPEVELPLLGDLIICRQVVEQEAIEQQKSAEEHWAHMVVHGCLHLLGYDHIEDDEAEEMESLETEILAELGYADPYLAEKE

SEQ ID NO: 25 - protein, 157 aa, Yersinia frederiksenii
MSQVILDLQIACADSQGLPTEADFQRWLEAVLPQFQEVAEVTIRLVDEAESHELNLTYRGKDKSTNVLSFPFEAPPEIELPLLGDLIICRQVVEQEAVEQDKALLAHWAHMVVHGSLHLLGYDHIVDDEAEEMESIETEIMQNLGYPDPYISEKDPE

SEQ ID NO: 26 - protein, 157 aa, Yersinia mollaretii
MSQVILDLQIACADSQGLPTEADFQRWLEAVLPQFQAVSEVTIRLVDEAESHELNLTYRGKDKSTNVLSFPFEAPPEIELPLLGDLIICRQVVEQEAVEQEKALFAHWAHMVVHGSLHLLGYDHIVDDEAEEMESIETEIMQSLGYPDPYISEKDPE

SEQ ID NO: 27 - protein, 153 aa, Vibrio fischeri
MSIELDLQIACENENGLPSEKELMTWLNAVIPQFQPQAELTIRIVDEKESHELNHEYRGKDKPTNVLSFPFEAPPGLELNLLGDLIICRQVVEEEAIEQNKPLLAHWAHMVVHGSLHLLGYDHIEDDEAEEMESLETELMQGMGFEDPYIAEK

SEQ ID NO: 28 - protein, 154 aa, Vibrio harveyi
MAIELDLQLAVENEEGLPSEQDFQLWLDKTIPLFQPQAELTIRIVDEQESHELNHEYRGKDKPTNVLSFPFEVPPGMEMDLLGDLIICRQVVEKEAVEQNKPLLAHWAHMVVHGSLHLLGYDHIEDDEAEEMESLETEIMQGMGYEDPYIAEKE

SEQ ID NO: 29 - protein, 159 aa, Pectobacterium atrosepticum
MSQVILDLQIASEQAQGLPEEKDFQRWLEGVLPQFQEVSEVTIRIVDEAESRDLNNTYRGKDKPTNVLSFPFEAPPEVELPLLGDLIICRQVVEREAVEQEKTVEEHWAHMVVHGSLHLLGYDHIEDSEAEEMEALETEIMQSMGYADPYLAEKDGLTE

SEQ ID NO: 30 - protein, 157 aa, Yersinia pseudotuberculosis
MSQVILDLQIACADSQGLPTEGDFQRWLEAVLPLFQPVSEVTIRLVDEAESHDLNLTYRGKDKSTNVLSFPFEAPPEIELPLLGDLIICRQVVEKEAIEQEKALLAHWAHMVVHGSLHLLGYDHIDDDEAEEMELIETEIMHGLGYPDPYISEKDPD

SEQ ID NO: 31 - protein, 153 aa, Vibrio splendidus
MSIELDLQIAVENEQGLPTEQDIQLWLDKTIPQFQENAELTVRIVDTEESHQLNHDYRGKDKPTNVLSFPFEVPPGMELDLLGDLIICRQVVEKEAEEQNKPLLAHWAHMVVHGSLHLLGYDHIEDDEAEEMESLETEIMQTMGFEDPYILEK

SEQ ID NO: 32 - protein, 154 aa, Vibrio cholerae
MSIELDLQLAVENEHGLPSEAEFALWLTRTITPFQAQAEVTVRIVDEAESHALNLNYRGKDKPTNVLSFPFEAPPGMEMDLLGDLVICRQVVEREAIEQNKPLQAHWAHMVVHGSLHLLGYDHIEDDEAEEMESLETEIMQEMGFTDPYLAEKE

SEQ ID NO: 33 - protein, 154 aa, Vibrio parahaemolyticus
MAIELDLQLAVENEEGLPSQQDFQLWLDKTIPLFQPQAEVTIRIVDEKESHALNHEYRGKDKPTNVLSFPFEAPPGMEIDLLGDLIICRQVVEKEAIEQNKPLLAHWAHMVVHGSLHLLGYDHIEDDEAEEMESLETEIMQGMGYEDPYIAEKE

SEQ ID NO: 34 - protein, 154 aa, Vibrio vulnificus
MAIELDLQLAVEDQNGLPSAQDFQTWLDKTIPPFQPQAEVTIRIVDSQESHQLNHDYRGKDKPTNVLSFPFEAPPGMEMDLLGDLVICRQVVEQEAIDQDKPLMAHWAHMVVHGSLHLLGYDHIEDDEAEEMESLETEIMQGMGFTDPYLAEKE

SEQ ID NO: 35 - protein, 154 aa, Vibrio alginolyticus
MAIELDLQLAVENEEGLPSQQDFQLWLDKTIPLFQPQAEVTIRIVDEQESHTLNHEYRGKDKPTNVLSFPFEAPPGMEIDLLGDLIICRQVVEKEAVEQSKPLLAHWAHMVVHGSLHLLGYDHIEDDEAEEMESLETEIMQGMGYEDPYIAEKE

SEQ ID NO: 36 - protein, 154 aa, Aeromonas salmonicida
MSVTLDLQLACADTDGLPGEAQLQGWLDGTILGFQEEAEVTVRIVDEAESRELNLTYRGKDKPTNVLSFPFEAPPGMELPLLGDLVICRQVVEQEATEQNKPLEAHWAHMVVHGSLHLLGYDHIEDDEAEEMEQLERDIMQELGFADPYLNDEE

SEQ ID NO: 37 - protein, 153 aa, Vibrionales bacterium
MSIELDLQLAVENEQGLPTEHDIQLWLDKTIPQFQKSAELTIRIVDTEESHQLNHEYRGKDKPTNVLSFPFEAPPGIELDFLGDLIICRQVVEKEAEEQNKPLLAHWAHMVVHGSLHLLGYDHIEDDEAEEMESLETEIMQAMGFEDPYILEK

SEQ ID NO: 38 - protein, 156 aa, Sodalis glossinidius
MSDVILDLQLACDEARGLPAEADFLRWLQGVLPLFRDCAEVTVRLVDEAESHELNMTYRGKDRPTNVLSFPFEAPPEVELPLLGDLVICHQVVEREAQQQEKALEAHWAHMVVHGSLHLLGYDHIQDEEALEMESLETEIMQKLGYPDPYLAEKEA

SEQ ID NO: 39 - protein, 158 aa, Pasteurella multocida
MKQVIIDLQLVCENTDNLPSEAQIQAWANRAIQPEFSDVEMTVRIVDEAESHDLNLTYRGKDKPTNVLSFPFECPDEVELSLLGDLVICRQVVEKEAEEQGKPLMAHWAHMVVHGCLHLLGYDHIEDAEAEEMEGLETEIMQSLGFDDPYLSEKEMHG

SEQ ID NO: 40 - protein, 154 aa, Vibrio sp.
MAIELDLQLAVENEEGLPSQQDFQLWLDKTIPLFQPQAEVTIRIVDEQESHTLNHEYRGKDKPTNVLSFPFEAPPGMEIDLLGDLIICRQVVEKEAIEQNKPLLAHWAHMVVHGSLHLLGYDHIEDDEAEEMESLETEIMQGMGYEDPYIAEKE

SEQ ID NO: 41 - protein, 153 aa, Vibrio sp.
MSIELDLQLAVENEQGLPTEQDIQLWLDKTIPQFQESAELTVRIVDTQESHQLNHDYRGKDKPTNVLSFPFEAPPGVELDLLGDLIICRQVVEKEAEEQSKPLLAHWAHMVVHGSLHLLGYDHIEDDEAEEMESLETEIMQTMGFEDPYILEK

SEQ ID NO: 42 - protein, 154 aa, Aeromonas hydrophila
MSVTLDLQLASASTDGLPSEAQLQGWLDGTILGFQQEAEVTVRIVDEAESNELNLTYRGKDKPTNVLSFPFEAPPGLELPLLGDLVICRQVVEREAAEQGKPLMAHWAHMVVHGSLHLLGYDHIEDDEAEEMETLERDIMQELGFADPYLNDEE

SEQ ID NO: 43 - protein, 157 aa, Yersinia pestis
MSQVILDLQIACADSQGLPTEGDFQRWLEAVLPLFQPVSEVTIRLVDEAESHDLNLTYRGKDKSTNVLSFPFEAPPEIELPLLGDLIICRQVVEKEAIEQEKALLAHWAHMVVHGSLHLLGYDHIDDEEAEEMELIETEIMHGLGYPDPYISEKDPD

SEQ ID NO: 44 - protein, 153 aa, Vibrio fischeri
MSIELDLQIACENENGLPSEKDLMTWLNAVIPQFQPQAELTIRIVDEKESHELNHEYRGKDKPTNVLSFPFEAPPGLELDLLGDLIICRQVVEEEAIEQNKPLLAHWAHMVVHGSLHLLGYDHIEDDEAEEMESLETELMQGMGFEDPYIAEK

SEQ ID NO: 45 - protein, 154 aa, Haemophilus somnus
MKNVIIDLQIASEDATNLPSVEQIQLWANAAIRAENSQPEMTVRIVDEEESHHLNLTYRGKDKPTNVLSFPFECPDEIELPLIGDLVICRQVVEREATEQEKPLMAHWAHMIVHGSLHLLGYDHIEDDEAEEMERLETEIMLSLGFTDPYIIEK

SEQ ID NO: 46 - protein, 156 aa, Mannheimia succiniciproducens
MMKTVTIDLQIASEDQSNLPTLEQFTLWATNAVRAEHFEPEITIRIVDEAESHELNFTYRGKDRPTNVLSFPFECPEEVELPLLGDLVICRQVVEREAQEQGKPLTAHWAHMVVHGSLHLLGYDHIEDDEAVEMESLETEIMTGLGFEDPYSYDEE

SEQ ID NO: 47 - protein, 160 aa, Mannheimia haemolytica
MENLYIDLQIACENTENLPSEQQFYTWVQKALAVEAKTDDFPESEITIRIVDEAESHELNLTYRGKDKPTNVLSFPFEAPEGIEMPLLGDLVICRQVMEKEAEEQGISAESHWAHLAIHGTLHLLGYDHIEEDEAVEMESLETEIMQSLGYEDPYLSEKI

SEQ ID NO: 48 - protein, 157 aa, Yersinia pseudotuberculosis
MSQVILDLQIACADSQGLPTEGDFQRWLEAVLPLFQPVSEVTIRLVDEAESHDLNLTYRGKDKSTNVLSFPFEAPPEIELPLLGDLIICRQVVEKEAIEQEKALLAHWAHMVVHGSLHLLGYDHIDDDEAEEMELIEIEIMHGLGYPDPYISEKDPD

SEQ ID NO: 49 - protein, 154 aa, Aggregatibacter actinomycetemcomitans
MGKMIIDLQIASADESGLPTAAQIEQWATAAVQPQSGEVEMTVRIVDETESHALNFNYRGKDHPTNVLSFPFECPDEVELPLLGDLVICRQVVEREAQEQEKPLIAHWAHMVVHGSLHLLGYDHIEDDEAEEMESLETQIMMGLGFVDPYLSEK

SEQ ID NO: 50 - protein, 154 aa, Haemophilus influenzae
MGSVLVDLQIATENIEGLPTEEQIVQWATGAVQPEGNEVEMTVRIVDEAESHELNLTYRGKDRPTNVLSFPFECPDEVELPLLGDLVICRQVVEREASEQEKPLMAHWAHMVVHGSLHLLGYDHIEDDEAEEMESLETQIMQGLGFDDPYLAEK

SEQ ID NO: 51 - protein, 156 aa, Actinobacillus succinogenes
MMKGVIIDLQIVSEDQTNLPTPEQFTQWATAAVRAEALEPEITIRIVDEAESHDLNLTYRGKDRPTNVLSFPFECPEEVELPLLGDLVICRQVVEREAEQQGKPLPAHWAHMVVHGCLHLLGYDHIEDDEAVEMESLETQIMTELGFEDPYSYDEI

SEQ ID NO: 52 - protein, 163 aa, Actinobacillus pleuropneumoniae
MTTPIIDLQIAAENSENLPSLAQFTQWVQRALAHEAQTEDFPETEITIRIVDEAESYELNLTYRGKDKPTNVLSFPFEVPEGIELPLLGDLIICRQVVEKEAQEQQISLESHWAHLAIHGTLHLLGYDHIEDAEAEEMEGLETEIMQSLGFEDPYISEKVIEE

SEQ ID NO: 53 - protein, 154 aa, Vibrio angustum
MAIYLDLQHATESLDGLPTEAEFQQWLNAAVIPFQADAEVTIRLVDEKESHALNLEYRGKDRPTNVLSFPFEAPPGMELELLGDLIICRQVVEKEALEQNKPLKAHWAHMVVHGSLHLLGYDHIEDEEAEEMEGLETEIMQNMGFVDPYISEKQ

SEQ ID NO: 54 - protein, 154 aa, Vibrio shilonii
MAIELDLQIAVESEQGLPSADEFQGWLDKTITPFQQDAELTIRIVDEEESQQLNRDYRGKDKPTNVLSFPFEAPPGVEMDLLGDMIICRQVVEKEAIEQSKPLIAHWAHMVVHGSLHLLGYDHIEDDEAEEMEALETEIMLDMGFDDPYLVEKE

SEQ ID NO: 55 - protein, 153 aa, Moritella sp.
MNTTLDLQIATENEQKLPTEAQLNAWLNAVVKRFQDSAEVTIRIVDNTESQQLNNDYRGKDKPTNVLSFPFEVPEGIELDLLGDLIICKQVVEREAKEQQKPLTAHWAHMVIHGTLHLLGYDHIIDEEAEEMEGLETEIMLELEFEDPYITEK

SEQ ID NO: 56 - protein, 154 aa, Photobacterium sp.
MAIYLDLQHATESLDGLPTEAEFQQWLNAAVIPFQADAEVTIRLVDEKESHTLNLEYRDKDRPTNVLSFPFEAPPGVELELLGDLIICRQVVEKEALEQNKPLKAHWAHMVVHGSLHLLGYDHIEDEEAEEMENLETEIMQNMGFVDPYISEKQ

SEQ ID NO: 57 - protein, 154 aa, Photobacterium profundum
MATYLDLQYATENQEGLPSEDKFQQWFEAAVTLFQSDAEITIRIVDVKESQALNLEYRGKDKPTNVLSFPFEAPPGIELDLLGDLIICRQVVEIEAKEQDKPLNAHWAHMVVHGTLHLLGYDHIEDEEAEEMEYLETEIIEKMGFADPFAAEKE

SEQ ID NO: 58 - protein, 158 aa, Haemophilus ducreyi
MNPIIDLQIASENSQGLPSAQQFSDWVKHALTYEAQTSNIPLTELTIRIVDEAESHHLNLTYRGKDKPTNVLSFPFEVPEGIELPLLGDLIICRQIVEKEALEQKISLDAHWAHLTIHGTLHLLGYDHIDEHEAEQMEGLESDIMQQLGFQDPYLAEK

SEQ ID NO: 59 - protein, 162 aa, Haemophilus parasuis
MNLYIDLQIASENTVGLPTAAQFQHWVDKALAMEAKTADYPETEITIRIVDEAESHELNLTYRGKDKPTNVLSFPFEMPEGIELPLLGDLIICRQVVEKEAVEQEKPLEAHWAHLAIHGTLHLLGYDHLTDEEAEEMESLETEIMQSLGFDDPYIAEKTIEE

SEQ ID NO: 60 - protein, 154 aa, Marinomonas sp.
MAYLELDLQIATEETANLPSEADFRLWVEKALPEVDEEFEVTIRIVDEEESHALNHEYRGKDKPTNVLSFPFEAPPGLELPLLGDLVICAQIVAKEAAEQNKELFHHWAHMTIHGILHLRGYDHINDDEADEMESIETELLASLSISDPYLIKE

SEQ ID NO: 61 - protein, 155 aa, Baumannia cicadellinicola
MSKIILDLQVACDHRCNLPSEDLFMYWLYMVLPLFRKKAEVTIRLVDEAESYNLNKIYRGQNHSTNVLSFPFKAPSPVKLVLLGDIIICRQVVEREAQEQNKILEAYWAHMVIHGSLHLLGYDHFIEQNAKKMEYLETKIMHKLGYLNPYETEIS

SEQ ID NO: 62 - protein, 156 aa, Shewanella frigidimarina
MSQQGISLDLDLQIAVDNPRLPTQAEFETWVRAAIGQTKPVVELTIRIVDIAESQQLNSTYRGKDKPTNVLSFPFEAPPEVELPLLGDLVICAPVVEQEAIEQNKPLIAHWAHMVIHGSLHLLGYDHIIDEEADEMESLETQLVEGLGFDNPYKEA

SEQ ID NO: 63 - protein, 158 aa, Shewanella sediminis
MNTSQAEIELDLDLQLAIENSQLPSKQNFELWVRTALPKTMAEAELTIRIVDEAESQELNSTYRGKDKPTNVLSFPFEAPPEIEIPLLGDLIICAPVVELEALQQNKPLQAHWAHMVVHGCLHLLGYDHINDAEAEEMESLETQLVESLGFNNPYKEQ

SEQ ID NO: 64 - protein, 153 aa, Shewanella denitrificans
MSLSLDLDLQIAVDSNQLPSQADFETWVRTALGNTLDTAELTIRLVEIAESQSLNHDYRGKDKPTNVLSFPFEAPPGMELPLLGDLVICVAVVEQEALEQNKPLQAHWAHMVIHGCLHLLGYDHIIDQEAEEMESLETQLIEGLGFSNPYKEA

SEQ ID NO: 65 - protein, 156 aa, Shewanella pealeana
MSDSQMVIDLDVQVAVEGFELPSQAELELWVKTALRDTMSEAELTIRIVDVEESQELNMTYRGKDKPTNVLSFPFEAPPGIELPLLGDLVICAAVVEQEAIDQNKPLLAHWAHMVVHGCLHLLGYDHIEDVEAEEMESLETQLIESLGYINPYKEQ

SEQ ID NO: 66 - protein, 152 aa, Pseudoalteromonas atlantica
MSAIVDLQVASDAANLPSADDFQRWLNAVLAHQQLSEHELTVRIVETQESQELNLTYRGKDKPTNVLSFPFEAPPGLSLNLLGDLVVCADVVAHEADEQHKKLHHHWAHMIVHGALHLLGFDHINDDEAKEMEALEVDILKQFSIDDPYQDQ

SEQ ID NO: 67 - protein, 158 aa, Shewanella woodyi
MSTSHTKLELALDLQIATDNKQLPTQQDFELWVRTALRNTMTEAELTVRVVDAEESQALNSTYRGKDKPTNVLSFPFEAPAEIELPLLGDLIICASVVEHEAIQQNKPLQAHWAHMVVHGCLHLLGYDHINDIEAEEMESIETQLIESLGFNNPYKEQ

SEQ ID NO: 68 - protein, 156 aa, Shewanella halifaxensis
MNDNQTVIDLDLQIAVEGFELPSQAELELWVKTALRDTMSEAELTIRIVDVEESQELNSTYRGKDKPTNVLSFPFEAPPGIELPLLGDLVICAAVVKQEAMDQNKPLIAHWAHMVVHGCLHLLGYDHIDDSEAEEMESLETHLIESLGYINPYKEQ

SEQ ID NO: 69 - protein, 154 aa, Marinomonas sp.
MDNLDLDLQLASQDTKGLPSEADFMQWIQPALPQNDTCFELTVRIVDEEESQNLNHQYRGKDKSTNVLSFPFEVPDGIELPLLGDLVICRQVVEKEAVEQNKKLFHHWAHMVIHGTLHLRGFDHIKEDDAQEMESLEISILGQLDIPNPYLINE

SEQ ID NO: 70 - protein, 158 aa, Shewanella benthica
MSLNHNNLELDLDLQVATDNERLPSQEDFELWVRIALRDTMNQAELTIRIVDEAESQTLNSSYRGKDKPTNVLSFPFEAPPEIDIPLLGDLIICAPVVEFEAKQQNKSLQSHWAHMVVHGCLHLLGYDHIQDAEAEEMESLETQLIESLGFNNPYKEQ

SEQ ID NO: 71 - protein, 163 aa, Reinekea sp.
MTELTLDLQIATDAENIPAEASFRQWVSAALSGYDREVEMSIRVVSAEESQDLNATYRGKDKPTNVLSFPFEAPPGVADAGIDRLLGDLVICAPVVEEEAQAQRKHLSHHWAHMVVHGTLHLLGYDHIEDDEAEQMEQAERTILAALSIPDPYQGEREEPSES

SEQ ID NO: 72 - protein, 157 aa, Shewanella loihica
MMPDSPIALHLDLQVATQAEQLPSQEEFESWVRLALGNVMPEAEMTIRLVDEAESQQLNHTYRGKDKPTNVLSFPFESPPEVELPLLGDLVICVPVVEQEAEIQGKSLTAHWAHMVVHGCLHLLGYDHIIDSEAEEMESLETQLIESLGFPNPYKEQ

SEQ ID NO: 73 - protein, 153 aa, Shewanella amazonensis
MALELALDLQFAVNPGNLPTEAEFETWVRVALGDTLDEAELTIRIVDATESQQLNRDYRGKDKPTNVLSFPFEAPPGMELPLLGDLVICASVVENEALEQHKALEAHWAHMVVHGCLHLLGYDHIDDAEAEEMEALETTLLTGLGYPDPYKEQ

SEQ ID NO: 74 - protein, 154 aa, Oceanobacter sp.
MIDVDVQIAIEGDNLPTPTQLQSWVTAALASLRPEAELSIRLVDNSESQSLNHEYRGKDKPTNVLSFPFEIPPELAEIENFTLIGDLVICHPVVCQEAAEQKKPLEHHYAHMVIHGCLHLLGYDHINEDEAEEMERLETDILATLNIPDPYIIN

SEQ ID NO: 75 - protein, 158 aa, Psychromonas ingrahamii
MQLYVDLQIACSDPNDLPMPASFEKWIEAAILGGSESHREEAELTVRIVDQDEIMQLNHQYRNISKTTNVLAFPFQNPPGLTLPLLGDLIICKEVVESEAKLQGKSLTAHWAHMSIHSTLHLLGYDHIEQAEAVEMESLETKLLTELGFTDPYLSEKE

SEQ ID NO: 76 - protein, 152 aa, Pseudoalteromonas tunicata
MTELDLQIASEFKDLPSFEQFQLWAHSTLDLFKEESELTIRIADEAESQQLNNDYRGKNKPTNVLSFPFEAPPGIELPLVGDLIICPQVVYQEALEQEKSFHDHFAHMVVHGCLHLLGFDHIEAEDAEEMESNEKEILAKLGISDPYRDDEE

SEQ ID NO: 77 - protein, 157 aa, Psychromonas sp.
MQLYIDLQIATMDEKALPSLALIETWIKEAIIKGSSITREEAELTVRIVDSVESQQLNGQYRHKDKPTNVLSFPFQAPMGIQLPLLGDLVICKQIVEQEALEQNKDLNAHWAHMLIHGSLHLLGFDHIIEQQALEMESLETKILVTLGFPPPYNETE

SEQ ID NO: 78 - protein, 157 aa, Marinobacter aquaeolei
MSKVTVDLQMAFDGTGVPSKTLFEAWAEAAWQGENPTEVTIRIVDNDESRELNHQYRGKDKPTNVLSFPFEAPAGITVPLAGDLVICAPVVEQEAREQNKDAVAHWAHMVVHGMLHLQGYDHIEVNEAEVMEALEIRLLAQLGFANPYEAEETEPDS

SEQ ID NO: 79 - protein, 151 aa, Neptuniibacter caesariensis
MSFDCDIQFEVEGNLPSESDFQRWVEAALSEFRDEGEICIRVVSPEESQELNHTYRDKDKPTNVLSFPFDIPEGIPIQLLGDLIICAEVVVTEASEQNKALFDHWAHMVVHGCLHLIGFDHIKDDEAEEMENLERQILASLDIADPYRDET

SEQ ID NO: 80 - protein, 149 aa, Saccharophagus degradans
MLTIDIQQASTADASQLPSDKQFEIWVEAALQQRMNEAELSIRIVDEDESQALNLQYRGKDKSTNVLSFPCELPDGVELPLLGDLVICAQVVAKEALEQGKLLHAHWAHMVVHGTLHLLGYDHIEDGEAEEMEAIEIQVLLELGYPNPY

SEQ ID NO: 81 - protein, 157 aa, Candidatus Blochmannia pennsylvanicus
MTSNQVIIDLQLACKNLHGLPNRKMFQSWVSAIFSIYKKKIELTVRIVDIKEMHYLNWYYLKKDCPTNVLSFPFTPPLGMKSPLLGDVVLCRQIIEYESKEKNVPGRSHWAHMIIHGSLHLLGYNHIVDKEAILMQRVERNILQKCGYRTCCHVAHR

SEQ ID NO: 82 - protein, 153 aa, Shewanella sp.
MSLDLALDVQYATASDYLPSEEQFALWVKTAIGNSMEQAELTIRIVDSRESQMLNSTYRGKDKPTNVLSFPFEAPPEIELPLLGDLVICATVVENEAREQDKTLEAHWAHMVVHGCLHLLGYDHIEDEEAEEMESLETQLIESLGFTDPYKEQ

SEQ ID NO: 83 - protein, 153 aa, Shewanella sp
MSLDLALDVQYATASDYLPSEDQFALWVKTAIGNSMEQAELTIRIVDARESQMLNSTYRGKDKPTNVLSFPFEAPPEIELPLLGDLVICAAVVENEAREQDKTLEAHWAHMVVHGCLHLLGYDHIEDEEAEEMESLETQLIESLGFTDPYKEQ

SEQ ID NO: 84 - protein, 166 aa, Homo sapiens
MSLVIRNLQRVIPIRRAPLRSKIEIVRRILGVQKFDLGIICVDNKNIQHINRIYRDRNVPTDVLSFPFHEHLKAGEFPQPDFPDDYNLGDIFLGVEYIFHQCKENEDYNDVLTVTATGLCHLLGFTHGTEAEWQQMFQKEKAVLDELGRRTGTRLQPLTRGLFGGS

SEQ ID NO: 85 - protein, 164 aa, Rattus norvegicus
MSLVIKNLQRVVPIRRMPLRRKMDLVRSILGVKKFDLGIICVDNKNIQNINRIYRNKNVPTDVLSFPFHENLKAGEFPQPRSPDDYNLGDVFLGVEYIFQYCKGNEDYYDVLTVTATHGLCHLLGFTHSSEAEWQKMYNQEKLVLEELSRHTGARLQPLSRGLY

SEQ ID NO: 86 - protein, 164 aa, Mus musculus
MSLVIKNLQRVVPIRRVPLRRKMDLVRSILGVKKFDLGIICVDNKTIQNINRIYRNKNVPTDVLSFSFHENLKAGEFPQPHSPDDYNLGDIFLGVEYILQHCRESEDYCDVLTVTATHGLCHLLGFTHSSKAEWQKMYNQEKLVLEELSRYTGARLQPLSRGLY

SEQ ID NO: 87 - protein, 165 aa, Ornithorhynchus anatinus
MSLVLRNLQRVIPIRRVPLRQKIETLRRILGVRRFDLGIVCVDNKNIQRVNNTYRQKNVPTDVLSFPFHENLKAGEMPQPRFPEDYNLGDVFLGVEYIFQQCQEGNEDFYGVLTVIAAHGLCHLLGYRHDTEAEWQEMYQKEKHILDELNKLTGTNLQPLTKNHF

SEQ ID NO: 88 - protein, 164 aa, Bos taurus
MSLALRNLQRAVPLRRALLRQRLQAVRGALGVRAFGLGVVCVDNRKIQQMNRIYREQDTPTDVLSFPFHENLKAGEIPQPDFPDDYNLGDIFLGVEFIFQQCKEDEDYYDILTVTATHGLCHLLGFTHSTEAEWQKMYQKEKQVLQELNKHMGTRLQPLSRGLF

SEQ ID NO: 89 - protein, 166 aa, Monodelphis domestica
MSLLLRNLQSAIPLRRAPLRARIELLRHILGIRRFDLGVVCVDNQGIQRLNRIYRQAHGPTDVLSFPFHEDLRAGAVPQPECPDDLNLGDIFLGVEYIFHQCQENGENYYDILTVTAAHGLCHLLGYKHNTAAEWREMFQKEKLTLEELNRVAGTSLQPLTKNLFG

SEQ ID NO: 90 - protein, 164 aa, Canis lupus familiaris
MSLVLRVPQRAVPVRRAPLRSRVELLRAVLGVRDFDLGLLCVDNEGMQRLNRAYRGDDRPTDVLSFPFHENVKAGELPRPRSRDDYNLGDIVLGVEYVFQRCRGDADYYDALTVTAAHGLCHLLGFTHSTEAEWRKMYQKEKQVLEELSRLTGTRLQPLSRGLF

SEQ ID NO: 91 - protein, 251 aa, Pan troglodytes
MWRPGRAELRGGRKRRLWRRPSPHAHRLAPGPRGPRCPLAPRKDLRRKHRLLPRHSALRTAGRKRRGPTSGSLSTTGFRPARCDPVPLPPTRSVAGLPVGRVRPLSRPLLGPDTGSVANIFKGLVILREMSLVIRNLQRVIPIRRAPLRSKIEIHLKAGEFPQPDFPDDYNLGDIFLGVEYIFHQCKENEDYNDILTVTATHGLCHLLGFTHGTEAEWQQMFQKEKAVLDELGRRTGTRLQPLTRGLFGGS

SEQ ID NO: 92 - protein, 163 aa, Danio rerio
MGVIVRNLQNVVPVRRARLRRDVEILRHIFGVQKFDMGIICVDNRKIQRINHTYRRRNQPTDVLSFPFYEDLRPGKVPCALQRDEYNLGDIFLGVEYVMQQCKETKQDLHQTLTVVTAHGICHLLGYRHETEEEWNEMQQKESYILSEFNRLTGSHLEPLTKR

SEQ ID NO: 93 - protein, 165 aa, Xenopus laevis
MSLILRNAQHAVPLYRAHLRFSLDIARSCLKVKNFDLGIICVNNARIQHINRVYRGQDSVTDVLSFPFHEDLNPSLLPIPATPDEYNLGDIYLGVAFIYEQCQKTQEDYRSILTITAVHGLCHLLGHKHNNPEKWKQMFEKETEILNEINRVTGSKLKPLSTNHY

SEQ ID NO: 94 - protein, 182 aa, Strongylocentrotus purpuratus
MSIIVRNFQKSVAFNLDKVKGDLKALRRILRVERFDVSVICVEDKDIRQMNKIYRATDEPTDVLSFPAHENLKPGRLPDPWSDLADLGDMFLGMGYIERDCTKHNADIDDVLPVIITHGLCHLIGYDHKTQEQWKMMHDRELQILEQFKKLTGQDLQPLTGRSDFLQAHHTDPQPHSQPTPA

SEQ ID NO: 95 - protein, 169 aa, Nematostella vectensis
MSVFVRNFQQRVLFSEALLERDARVLVQLLKADRFDVSIVCAGGKRIKSLNLKYRRRNVQTDVLAFPYFENMKPGVLPNPKLQDDWNLGDVILGMPVIHQDCQEDNKSVQEYLPVLITHGLCHLLGYTHDTEQNLQQMHKKEKEVLSGFNQVTGYNTKPLLPAITPSDR

SEQ ID NO: 96 - protein, 11 aa, Artificial sequence
  synthetic polypeptide
HXXXHXXXXXH

SEQ ID NO: 97 - DNA, 18 bp, Artificial sequence
  synthetic oligonucleotide
cgacttgcatgtgttagg

SEQ ID NO: 98 - DNA, 26 bp, Artificial sequence
  synthetic oligonucleotide
gggcatccaccgtgtacgcttagtcg

SEQ ID NO: 99 - RNA, 50 nt, Artificial sequence
  synthetic oligonucleotide
guuggaucaccuccuuaccuuaaagaagcguucuuugaagugcucacaca

SEQ ID NO: 100 - RNA, 50 nt, Artificial sequence; identical to SEQ ID NO: 99
  synthetic oligonucleotide

SEQ ID NO: 101 - RNA, 27 nt, Artificial sequence
  synthetic oligonucleotide
guacgcuucuuugagcaucaaacuuuu

SEQ ID NO: 102 - RNA, 50 nt, Artificial sequence; identical to SEQ ID NO: 99
  synthetic oligonucleotide

SEQ ID NO: 103 - RNA, 27 nt, Artificial sequence
  Synthetic oligonucleotide
auaaccuacucaggaggugauccaacc

SEQ ID NO: 104 - DNA, 25 bp, Artificial sequence
  synthetic oligonucleotide
ggggtcaggtgggaccaccgcgcta

SEQ ID NO: 105 - RNA, 30 nt, Artificial sequence
  synthetic oligonucleotide
gguuggaucaccuccuuaccuuaaagaagc

SEQ ID NO: 106 - RNA, 20 nt, Artificial sequence
  synthetic oligonucleotide
gguuggaucaccuccuuacc

SEQ ID NO: 107 - RNA, 20 nt, Artificial sequence
  synthetic oligonucleotide
gguaaggaggugauccaacc

SEQ ID NO: 108 - RNA, 30 nt, Artificial sequence; identical to SEQ ID NO: 105
  synthetic oligonucleotide

SEQ ID NO: 109 - RNA, 15 nt, Artificial sequence
  synthetic oligonucleotide
ggaggugauccaacc

SEQ ID NO: 110 - RNA, 30 nt, Artificial sequence; identical to SEQ ID NO: 105
  synthetic oligonucleotide

SEQ ID NO: 111 - RNA, 20 nt, Artificial sequence
  synthetic oligonucleotide
gguaaggaggugauccaacc

SEQ ID NO: 112 - RNA, 30 nt, Artificial sequence; identical to SEQ ID NO: 105
  gguuggauca ccuccuuacc uuaaagaagc

SEQ ID NO: 113 - RNA, 17 nt, Artificial sequence
  gguuggauca ccuccuuacc uuaaagaagc
gcuucuuuaagguaagg

SEQ ID NO: 114 - RNA, 21 nt, Artificial sequence
  synthetic oligonucleotide
agaaauucuucaaaaauuucu

SEQ ID NO: 115 - protein, 168 aa, Sinorhizobium meliloti
MTALDIQISVEAGDWPPEDELQSFCERVLEAAADFLAREENQPLPAQAAELSLVFTDDQSIRAINAEWRGQDKATNVLSFPAFPVTPGRMPGPMLGDIVVAHETLRREAAELEKPFDAHLTHLLVHGFLHLFGYDHIEDDEAERMEGLETRILARLGLSDPYGDQPPH

SEQ ID NO: 116 - protein, 155 aa, Escherichia coli; identical to SEQ ID NO: 1

SEQ ID NO: 117 - protein, 150 aa, Aquifex aeolicus
MSSTKRQKNRVLVKLKKRKVRKDKIEKWAELALSALGLNNVELSVYITDDQEIRELNKTYRKKDKPTDVLSFPMGEEFGGYKILGDVVISQDTAERQARELGHSLEEEVKRLIVHGIVHLLGYDHEKGGEEEKKFRELENYVLSKLSKAL

SEQ ID NO: 118 - protein, 157 aa, Bacillus subtilis
MSLLIDIVDETGSVSEEMLKEVENLLQFAAEREGVQDQAEVSVTIVSNDDIHQINKEYRGKDAPTDVISFALEEEGEGEIEIVGAEMPPVLGNIIISADRTREQAEEYNHSFKRELGFLAVHGFLHLLGYDHMTKEEEEEMFTKQKELLDAYGLKRS

SEQ ID NO: 119 - protein, 164 aa, Canis familiaris; identical to SEQ ID NO: 90

SEQ ID NO: 120 - protein, 165 aa, Xenopus laevis; identical to SEQ ID NO: 93

SEQ ID NO: 121 - protein, 166 aa, Homo sapiens; identical to SEQ ID NO: 84

SEQ ID NO: 122 - protein, 96 aa, Artificial sequence
  synthetic polypeptide
MSLVIRIQVLKGLLELLGVQELSLVVDNIINKYRGKDKPTDVLSFPFEGPGEMLGDIVVAELQAEFLTLAVHGILHLLGYDHDDEEMFKEILLGKP

(19) United States
(12) Patent Application Publication
(10) Pub. No.: US 2010/0222410 A1
(43) Pub. Date: Sep. 2, 2010

(54) Nuclease compositions and methods

(75) Inventors: Bryan W. Davies, Allston, MA (US); Caroline Koehrer, Somerville, MA (US); Asha I. Jacob, Somerville, MA (US); Uttam L. RajBhandary, Lexington, MA (US); Graham C. Walker, Cambridge, MA (US); Brenda Kathleen Minesinger, Somerville, MA (US); Shree Prakash Pandey, Boston, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/460,446

(22) Filed: Jul. 17, 2009

Related U.S. Application Data
(60) Provisional application No. 61/082,069, filed on Jul. 18, 2008.

(51) Int. Cl. A61K 31/7088; C12N 9/48; G01N 33/554; A61K 31/165; A61K 31/546; A61K 31/13; A61K 31/4164; A61K 31/421; A61K 31/426; A61K 31/18; A61P 31/04

(52) U.S. Cl. 514/44 A; 435/212; 435/7.32; 514/620; 514/207; 514/662; 514/386; 514/378; 514/370; 514/604

(57) ABSTRACT

The present invention generally relates to various nucleases and uses thereof, and in some cases, to the UPF0054 protein superfamily. Members of the UPF0054 protein superfamily, such as the E. coli protein YbeY, may possess RNase activity and may be involved in certain important cellular processes. Disruption of YbeY activity can lead to increased sensitivity to antibiotics. Accordingly, certain embodiments of the invention are directed to systems and methods for screening target compounds for activity against UPF0054 superfamily proteins. In some embodiments, the screening method allows to target compositions to be determined that show selective activity against UPF0054 superfamily proteins. Other embodiments of the invention provide for nucleases capable of site-specific cleavage of nucleic acids.

### RELATED APPLICATIONS

This application claims the benefit of U.S. Provisional Patent Application Ser. No. 61/082,069, filed Jul. 18, 2008, entitled “Nuclease Compositions and Methods,” by Davies, et al., incorporated herein by reference.

### GOVERNMENT FUNDING

Research leading to various aspects of the present invention were sponsored, at least in part, by the National Institutes of Health, Grant No. GM031030. The U.S. Government has certain rights in the invention.

### FIELD OF THE INVENTION

The present invention generally relates to various nucleases and uses thereof, and in some cases, to the UPF0054 protein superfamily.

### BACKGROUND OF THE INVENTION

Ribosome maturation is a complex procedure requiring precise processing and modification of rRNA and r-proteins to form fully functional ribosomes. In bacteria, analysis of the effects of immature rRNA species on translation and cell survival suggests that maturation of the 16S rRNA 3′ terminus may be important in rRNA maturation, although the enzyme that perform this function has remained elusive.

Combined with transcription, translation allows for the expression of proteins encoded by DNA. Translation, a complex process performed by the ribosome and its associated factors, has been studied for more than half a century, and new factors required for translation and ribosome maturation are still being discovered. In bacteria, the ribosome is composed of two subunits; a large 50S subunit and smaller 30S subunit. The 50S subunit comprises of 23S rRNA, 5S rRNA, and 33 ribosomal-proteins, whereas the 30S subunit contains 16S rRNA and 21 ribosomal-proteins. The 50S and 30S subunits combine to form an active 70S ribosome that is competent for translation. Although reconstitution of active 30S and 50S subunits has been performed in vitro using only mature rRNA and ribosomal proteins, it is recognized that several additional factors are required for 50S and 30S formation under physiological conditions.

Ribosome maturation occurs in a cooperative and ordered fashion. The 16S, 23S, and 5S rRNA are co-transcribed as part of a large rRNA precursor. Before transcription is complete, ribosomal proteins associate with rRNA in a cooperative order forming ribonucleoprotein complexes that are acted on by RNaseIII. RNaseIII cleaves the original transcript into precursors that will become mature 16S, 23S and 5S rRNA.

### SUMMARY OF THE INVENTION

The present invention generally relates to various nucleases and uses thereof, and in some cases, to the UPF0054 protein superfamily. The subject matter of the present invention involves, in some cases, interrelated products, alternative solutions to a particular problem, and/or a plurality of different uses of one or more systems and/or articles.

In one aspect, the invention is generally directed to a method. The method, in one set of embodiments, includes an act of cleaving a polynucleotide by exposing the polynucleotide to an isolated UPF0054 superfamily protein. The method, in another set of embodiments, includes an act of determining affinity of a polynucleotide for an isolated UPF0054 superfamily protein.

The method, in yet another set of embodiments, includes an act of purifying an isolated UPF0054 superfamily protein from a solution containing the isolated protein, without exposing the isolated protein to a Mg²⁺ concentration of greater than about 5 mM, wherein the solution contains cell lysate. In still another set of embodiments, the method includes acts of purifying an isolated protein from a solution without exposing the isolated protein to a Mg²⁺ concentration of greater than about 5 mM.

In one set of embodiments, the method includes acts of providing a first cell able to express a first UPF0054 superfamily protein, providing a second cell able to express a second UPF0054 superfamily protein distinguishable from the first protein, exposing at least one of the first and second cells to an agent, and determining growth rates of the first and second cells. In another set of embodiments, the method includes acts of providing a first cell and a second cell each having a growth rate in the presence of an antibiotic, where the first cell is able to express a first UPF0054 superfamily protein and the second cell is able to express a second UPF0054 superfamily protein distinguishable from the first protein, exposing the first and second cells to an agent, and determining growth rates of the first and second cells after exposure to the agent.

In one set of embodiments, the method includes acts of providing a cell having a growth rate, where the cell is able to express a non-endogenous UPF0054 superfamily protein, exposing the cell to an agent; and determining a change in the growth rate of the cell after exposure to the agent.

In another set of embodiments, the method includes acts of exposing a bacteria or other cell to an inhibitor of a member of UPF0054 protein superfamily, and exposing the bacteria or other cell to an antibiotic. In still another set of embodiments, the method includes acts of administering, to a subject, an inhibitor of a member of the UPF0054 protein superfamily, and administering an antibiotic to the subject. The antibiotic may be different from the inhibitor, in some cases.

The invention, in another aspect, is directed to a composition. In one set of embodiments, the composition includes a polynucleotide bound to an isolated UPF0054 superfamily protein. The composition, in another set of embodiments, includes an isolated UPF0054 superfamily protein contained in a solution having less than about 5 mM of Mg²⁺.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIGS. 1A-1J generally show phenotypic analysis of the E. coli ΔybeY mutant, in accordance with various embodiments of the invention;

FIGS. 2A-2D generally show rRNA analysis of MC4100 and the ΔybeY mutant, in accordance with various embodiments of the invention;

FIGS. 3A-3N generally show YbeY RNase activity, in accordance with various embodiments of the invention;

FIGS. 4A-4H generally show comparison of rRNA from the ΔybeY mutant and seven well-characterized E. coli RNase mutants, in accordance with various embodiments of the invention;

FIGS. 5A-5B generally show time course monitoring of the maturation of 16S rRNA 5′ and 3′ termini in the ΔybeY mutant following induction of ybeY from an arabinose inducible vector, in accordance with various embodiments of the invention;

FIG. 5C shows a plot showing the growth of MC4100, the ΔybeY mutant and several ΔybeY mutant double mutants, in accordance with various embodiments of the invention;

FIG. 5D shows FLAG-tagged YbeY immunoprecipitated from an MC4100 whole cell lysate, in accordance with various embodiments of the invention;

FIGS. 6A-6I generally show characterization of the human UPF0054 homolog C21orf57, in accordance with various embodiments of the invention;

FIG. 7 shows a genetic pathway identifying known RNases required for normal rRNA maturation, in accordance with various embodiments of the invention;

FIG. 8 shows sequence alignment of UPF0054 homologs from bacteria and eukaryotes, in accordance with various embodiments of the invention;

FIGS. 9A-9C show various detailed diagrams of 16S rRNA 3′ termini mimics, in accordance with various embodiments of the invention; and FIG. 10 shows YbeY RNase activity against 16S rRNA 3′ terminus mimics, in accordance with various embodiments of the invention.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### BRIEF DESCRIPTION OF THE SEQUENCES

SEQ ID NO: 1 is Escherichia coli K12 UPF0054 having the sequence MSQVILDLQLACEDNSGLPEESQFQTWLNAVIPQFQEESEVTIRVVDTAESHSLN LTYRGKDKPTNVLSFPFEVPPGMEMSLLGDLVICRQVVEKEAQEQGKPLEAHW AHMVVHGSLHLLGYDHIEDDEAEEMEALETEIMLALGYEDPYIAEKE;

SEQ ID NO: 2 is Shigella flexneri UPF0054 having the sequence MSQVILDLQLACEDNSGLPEESQFQTWLNAVIPQFQEESEVTIRVVDTAESHSLN LTYRGKDKPTNVLSFPFEVPPGMEMSLLGDLVICRQVVEKEAQEQGKPLEAHW AHMVVHGSLHLLGYDHIEDDEAEEMEALETEIMLALGYEDPYIAEKE;

SEQ ID NO: 3 is Shigella sonnei Ss046 UPF0054 having the sequence MSQVILDLQLACEDNSGLPEESQFQTWLNAVIPQFQEESEVTIRVVDTAESHSLN LTYRGKDKPTNVLSFPFEVPPGMEMSLLGDLVICRQVVEKEAQEQGKPLEAHW AHMVVHGSLHLLGYDHIEDDEAEEMEALETEIMLALGYEDPYIAEKE;

SEQ ID NO: 4 is Shigella boydii Sb227 UPF0054 having the sequence MSQVILDLQLACEDNSGLPEESQFQTWLNAVIPQFQEESEVTIRVVDTAESHSLN LTYRGKDKPTNVLSFPFEVPPGMEMSLLGDLVICRQVVEKEAQEQGKPLEAHW AHMVVHGSLHLLGYDHIEDDEAEEMEALETEIMLALGYEDPYIAEKE;

SEQ ID NO: 5 is Shigella dysenteriae 1012 UPF0054 having the sequence MSQVILDLQLACEDNSGLPEESQFQTWLNAVIPQFQEESEVTVRVVDTAESHSLN LTYRGKDKPTNVLSFPFEVPPGMEMSLLGDLVICRQVVEKEAQEQGKPLEAHW AHMVVHGSLHLLGYDHIEDDEAEEMEALETEIMLALGYEDPYIAEKE;

SEQ ID NO: 6 is Escherichia coli 0157:H7 UPF0054 having the sequence MSQVILDLQLACEDNSGLPEESQFQTWLNAVIPQFQEESEVTIRVVDTAESHSLN LTYRGKDKPTNVLSFPFEVPPGMEMSLLGDLVICRQMVEKEAQEQGKPLEAHW AHMVVHGSLHLLGYDHIEDDEAEEMEALETEIMLALGYEDPYIAEKE;

SEQ ID NO: 7 is Escherichia albertii TW07627 UPF0054 having the sequence MSQVILDLQLACEDNSGQPEESQFQTWLNAVIPQFQEESEVTIRLVDTAESHSLN LTYRGKDKPTNVLSFPFEVPPGMEMSLLGDLVICRQVVEQEALEQGKPLEAHW AHMVVHGSLHLLGYDHIEDDEAEEMEALETEIMLALGYEDPYIAEKE;

SEQ ID NO: 8 is Salmonella enterica subsp. enterica serovar Typhi str. CT18 UPF0054 having the sequence MSQVILDLQLACENHAGLPDEAQFQRWLDGVIPQFQEEAEVTIRLVDEAESHDL NLTYRGKDKPTNVLSFPFEAPPGIEMPLLGDLIICRQVVEQEAQEQSKPLEAHWA HMVVHGSLHLLGYDHIDDDEAEEMESLETEIMLAMGYEDPYIAEKIAE;

SEQ ID NO: 9 is Salmonella typhi UPF0054 having the sequence MSQVILDLQLACENHAGLPDEAQFQRWLDGVIPQFQEEAEVTIRLVDEAESHDL NLTYRGKDKPTNVLSFPFEAPPGIEMPLLGDLIICRQVVEQEAQEQSKPLEAHWA HMVVHGSLHLLGYDHIDDDEAEEMESLETEIMLAMGYEDPYIAEKIAE;

SEQ ID NO: 10 is Salmonella choleraesuis UPF0054 having the sequence MSQVILDLQLACENHAGLPDEAQFQRWLDGVIPQFQEEAEVTIRLVDEAESHDL NLTYRGKDKPTNVLSFPFEAPPGIEMPLLGDLIICRQVVEQEAQEQSKPLEAHWA HMVVHGSLHLLGYDHIDDDEAEEMESLETEIMLAMGYEDPYIAEKIAE;

SEQ ID NO: 11 is Salmonella paratyphi UPF0054 having the sequence MSQVILDLQLACENHAGLPDEAQFQRWLDGVIPQFQEEAEVTIRLVDEAESHDL NLTYRGKDKPTNVLSFPFEAPPGIEMPLLGDLIICRQVVEQEAQEQSKPLEAHWA HMVVHGSLHLLGYDHIDDDEAEEMESLETEIMLAMGYEDPYIAEKITE;

SEQ ID NO: 12 is Enterobacter sakazakii ATCC BAA-894 UPF0054 having the sequence MSQVILDLQVACEDTTGLPDEAQFQTWLNAVVPQFQEESEVTVRLVDEAESHD LNLTYRGMDKPTNVLSFPFEAPPGIDMPLLGDLIICRQVVEREAREQEKPLEAHW AHMVVHGSLHLLGYDHIEDDEAEEMEGIETEIMLALGFDDPYIAEKA;

SEQ ID NO: 13 is Salmonella typhimurium UPF0054 having the sequence MSQVILDLQLACENHAGLPDEAQFQRWLDGVIPQFQEEAEVTIRLVDEAESHDL NLTYRGKDKPTNVLSFPFEAPAGIEMPLLGDLIICRQVVEQEAQEQSKPLEAHWA HMVVHGSLHLLGYDHIDDDEAEEMESLETEIMLAMGYEDPYIAEKIAE;

SEQ ID NO: 14 is Klebsiella pneumoniae subsp. pneumoniae MGH 78578 UPF0054 having the sequence MSQVILDLQLACEETSGLPDEALFQRWVDAVIPPFQEESELTIRLVDVAESHELN LTYRGKDKPTNVLSFPFEAPPGIEMPLLGDLIICRQVVEQEASEQGKPLEAHWAH MVVHGSLHLLGYDHIEDDEAEEMEGLETEIMLALGYEDPYISEKIAE;

SEQ ID NO: 15 is Citrobacter koseri ATCC BAA-895 UPF0054 having the sequence MSQVILDLQLACEDHSGLPEESQFQTWLNAVIPQFQEESEVTIRLVDEAESHDLN LTYRGKDKPTNVLSFPFEAPPGMEMPLLGDLIICRQVVEQEAKEQDKPLEAHWA HMVVHGSLHLLGYDHIEDDEAEEMEALETEIMLALGYEDPYISEKD;

SEQ ID NO: 16 is Enterobacter sp. 638 UPF0054 having the sequence MSQVILDLQLACEDNSGLPEESQFQKWLDAVIPQFQEESEVTIRVVDEAESHELN LTYRGKDKPTNVLSFPFEAPPGIELPLLGDLIICRQVVEQEAKEQQKPLDAHWAH MVIHGSLHLLGYDHIEDEEAEEMESLETEIMLALGYEDPYIAEKE;

SEQ ID NO: 17 is Serratia proteamaculans 568 UPF0054 having the sequence MSQVILDLQLACADNSGLPDEATFQRWLEGVLPQFQEEAEVTIRLVDEAESNEL NLTYRGMDKPTNVLSFPFEAPPGIELPLLGDLIICRQVVEREAAEQDKALEAHWA HMVVHGSLHLLGYDHIEDDEAEEMESLETEIMHGLGYPDPYLAEKDPL;

SEQ ID NO: 18 is Erwinia tasmaniensis UPF0054 having the sequence MSGVILDLQLACENEQGLPAETDFQRWLEAVLPQFQPESEVTIRLVDEAESRELN HTYRSKDKPTNVLSFPFEAPPGIELPLLGDLIICRQVVEQEAVEQGKTREAHWAH MVIHGSLHLLGYDHIEDDEAEEMESLETEIMLALGYPDPYISEKE;

SEQ ID NO: 19 is Yersinia enterocolitica subsp. enterocolitica 8081 UPF0054 having the sequence MSQVILDLQIACADSQGLPTEADFQHWLEAVLPQFQEVSEVTIRVVDEAESHEL NLTYRGKDKPTNVLSFPFEAPPEIELPLLGDLIICRQVVEQEAVEQEKALLAHWA HMVVHGSLHLLGYDHIVDDEAEEMESIETEIMQSLGYPDPYISEKDPE

SEQ ID NO: 20 is Photorhabdus luminescens subsp. laumondii TTO1 UPF0054 having the sequence MSSVILDLQIACEHSQGLPKETLFQHWLDGVLPQFQSESEVTIRIVDEAESHDLNL TYRGKNKPTNVLSFPFEAPPEIDLPLLGDLIICRQVVEKEAEEQQKTIEEHWAHM VVHGCLHLLGYDHIEDDEAEEMESLETEIMQKLGYADPYLAEKE;

SEQ ID NO: 21 is Yersinia intermedia ATCC 29909 UPF0054 having the sequence MSQVILDLQIACADSQGLPAEADFQRWLEAVLPQFQDVAEVTVRLVDEAESHEL NLTYRGKDKPTNVLSFPFEAPPEIELPLLGDLIICRQVVEQEAIEQEKALLAHWAH MVVHGSLHLLGYDHIVDDEAEEMESIETEIMQSLGYPDPYISEKDPV; SEQ ID NO: 22 is Yersinia bercovieri ATCC 43970 UPF0054 having the sequence MSQVILDLQIACANSQGLPTEADFQQWLEAVLPQFQEVSEVTIRLVDEAESHQL NLTYRGKDKSTNVLSFPFEAPPEIELPLLGDLIICRQVVEQEAVEQEKALFAHWA HMVVHGSLHLLGYDHIVDDEAEEMESIETEIMQSLGYPDPYISEKESD;

SEQ ID NO: 23 is Vibrio vulnificus YJOI6 UPF0054 having the sequence MSRRLTYLAVQNRAFVMPSKCSMKWKRSALTFSNPMTSFATQSSRASSMPTRS GKHKTKKSVKSLNNVAEKSAMPNCLKPQKPSFLHKSARIRKPHMAIELDLQLAV EDQNGLPSAQDFQTWLDKTIPPFQPQAEVTIRIVDSQESHQLNHDYRGKDKPTN VLSFPFEAPPGMEMDLLGDLVICRQVVEQEAIEQDKPLMAHWAHMVVHGSLHL LGYDHIEDDEAEEMESLETEIMQGMGFTDPYLAEKE;

SEQ ID NO: 24 is Providencia stuartii ATCC 25827 UPF0054 having the sequence MSEVILDLQLACEETTGLPDEALFQRWLEAVLPKFQPQSEVTIRIVDEEESHHLN LTYRGKDKPTNVLSFPFEAPPEVELPLLGDLIICRQVVEQEAIEQQKSAEEHWAH MVVHGCLHLLGYDHIEDDEAEEMESLETEILAELGYADPYLAEKE;

SEQ ID NO: 25 is Yersinia frederiksenii ATCC 33641 UPF0054 having the sequence MSQVILDLQIACADSQGLPTEADFQRWLEAVLPQFQEVAEVTIRLVDEAESHEL NLTYRGKDKSTNVLSFPFEAPPEIELPLLGDLIICRQVVEQEAVEQDKALLAHWA HMVVHGSLHLLGYDHIVDDEAEEMESIETEIMQNLGYPDPYISEKDPE;

SEQ ID NO: 26 is Yersinia mollaretii ATCC 43969 UPF0054 having the sequence MSQVILDLQIACADSQGLPTEADFQRWLEAVLPQFQAVSEVTIRLVDEAESHELN LTYRGKDKSTNVLSFPFEAPPEIELPLLGDLIICRQVVEQEAVEQEKALFAHWAH MVVHGSLHLLGYDHIVDDEAEEMESIETEIMQSLGYPDPYISEKDPE;

SEQ ID NO: 27 is Vibrio fischeri ES 114 UPF0054 having the sequence MSIELDLQIACENENGLPSEKELMTWLNAVIPQFQPQAELTIRIVDEKESHELNHE YRGKDKPTNVLSFPFEAPPGLELNLLGDLIICRQVVEEEAIEQNKPLLAHWAHMV VHGSLHLLGYDHIEDDEAEEMESLETELMQGMGFEDPYIAEK;

SEQ ID NO: 28 is Vibrio harveyi HY01 UPF0054 having the sequence MAIELDLQLAVENEEGLPSEQDFQLWLDKTIPLFQPQAELTIRIVDEQESHELNHE YRGKDKPTNVLSFPFEVPPGMEMDLLGDLIICRQVVEKEAVEQNKPLLAHWAH MVVHGSLHLLGYDHIEDDEAEEMESLETEIMQGMGYEDPYIAEKE;

SEQ ID NO: 29 is Pectobacterium atrosepticum UPF0054 having the sequence MSQVILDLQIASEQAQGLPEEKDFQRWLEGVLPQFQEVSEVTIRIVDEAESRDLN NTYRGKDKPTNVLSFPFEAPPEVELPLLGDLIICRQVVEREAVEQEKTVEEHWAH MVVHGSLHLLGYDHIEDSEAEEMEALETEIMQSMGYADPYLAEKDGLTE;

SEQ ID NO: 30 is Yersinia pseudotuberculosis IP 32953 UPF0054 having the sequence MSQVILDLQIACADSQGLPTEGDFQRWLEAVLPLFQPVSEVTIRLVDEAESHDLN LTYRGKDKSTNVLSFPFEAPPEIELPLLGDLIICRQVVEKEAIEQEKALLAHWAH MVVHGSLHLLGYDHIDDDEAEEMELIETEIMHGLGYPDPYISEKDPD;

SEQ ID NO: 31 is Vibrio splendidus 12B01 UPF0054 having the sequence MSIELDLQIAVENEQGLPTEQDIQLWLDKTIPQFQENAELTVRIVDTEESHQLNH DYRGKDKPTNVLSFPFEVPPGMELDLLGDLIICRQVVEKEAEEQNKPLLAHWAH MVVHGSLHLLGYDHIEDDEAEEMESLETEIMQTMGFEDPYILEK;

SEQ ID NO: 32 is Vibrio cholerae 1587 UPF0054 having the sequence MSIELDLQLAVENEHGLPSEAEFALWLTRTITPFQAQAEVTVRIVDEAESHALNL NYRGKDKPTNVLSFPFEAPPGMEMDLLGDLVICRQVVEREAIEQNKPLQAHWA HMVVHGSLHLLGYDHIEDDEAEEMESLETEIMQEMGFTDPYLAEKE;

SEQ ID NO: 33 is Vibrio parahaemolyticus UPF0054 having the sequence MAIELDLQLAVENEEGLPSQQDFQLWLDKTIPLFQPQAEVTIRIVDEKESHALNH EYRGKDKPTNVLSFPFEAPPGMEIDLLGDLIICRQVVEKEAIEQNKPLLAHWAHM VVHGSLHLLGYDHIEDDEAEEMESLETEIMQGMGYEDPYIAEKE;

SEQ ID NO: 34 is Vibrio vulnificus UPF0054 having the sequence MAIELDLQLAVEDQNGLPSAQDFQTWLDKTIPPFQPQAEVTIRIVDSQESHQLNH DYRGKDKPTNVLSFPFEAPPGMEMDLLGDLVICRQVVEQEAIDQDKPLMAHWA HMVVHGSLHLLGYDHIEDDEAEEMESLETEIMQGMGFTDPYLAEKE;

SEQ ID NO: 35 is Vibrio alginolyticus 12G01 UPF0054 having the sequence MAIELDLQLAVENEEGLPSQQDFQLWLDKTIPLFQPQAEVTIRIVDEQESHTLNH EYRGKDKPTNVLSFPFEAPPGMEIDLLGDLIICRQVVEKEAVEQSKPLLAHWAH MVVHGSLHLLGYDHIEDDEAEEMESLETEIMQGMGYEDPYIAEKE;

SEQ ID NO: 36 is Aeromonas salmonicida subsp. salmonicida A449 UPF0054 having the sequence MSVTLDLQLACADTDGLPGEAQLQGWLDGTILGFQEEAEVTVRIVDEAESRELN LTYRGKDKPTNVLSFPFEAPPGMELPLLGDLVICRQVVEQEATEQNKPLEAHWA HMVVHGSLHLLGYDHIEDDEAEEMEQLERDIMQELGFADPYLNDEE;

SEQ ID NO: 37 is Vibrionales bacterium SWAT-3 UPF0054 having the sequence MSIELDLQLAVENEQGLPTEHDIQLWLDKTIPQFQKSAELTIRIVDTEESHQLNHE YRGKDKPTNVLSFPFEAPPGIELDFLGDLIICRQVVEKEAEEQNKPLLAHWAHM VVHGSLHLLGYDHIEDDEAEEMESLETEIMQAMGFEDPYILEK;

SEQ ID NO: 38 is Sodalis glossinidius str. ‘morsitans’ UPF0054 having the sequence MSDVILDLQLACDEARGLPAEADFLRWLQGVLPLFRDCAEVTVRLVDEAESHEL NMTYRGKDRPTNVLSFPFEAPPEVELPLLGDLVICHQVVEREAQQQEKALEAHW AHMVVHGSLHLLGYDHIQDEEALEMESLETEIMQKLGYPDPYLAEKEA;

SEQ ID NO: 39 is Pasteurella multocida UPF0054 having the sequence MKQVIIDLQLVCENTDNLPSEAQIQAWANRAIQPEFSDVEMTVRIVDEAESHDL NLTYRGKDKPTNVLSFPFECPDEVELSLLGDLVICRQVVEKEAEEQGKPLMAHW AHMVVHGCLHLLGYDHIEDAEAEEMEGLETEIMQSLGFDDPYLSEKEMHG;

SEQ ID NO: 40 is Vibrio sp. Ex25 UPF0054 having the sequence MAIELDLQLAVENEEGLPSQQDFQLWLDKTIPLFQPQAEVTIRIVDEQESHTLNH EYRGKDKPTNVLSFPFEAPPGMEIDLLGDLIICRQVVEKEAIEQNKPLLAHWAHM VVHGSLHLLGYDHIEDDEAEEMESLETEIMQGMGYEDPYIAEKE;

SEQ ID NO: 41 is Vibrio sp. MED222 UPF0054 having the sequence MSIELDLQLAVENEQGLPTEQDIQLWLDKTIPQFQESAELTVRIVDTQESHQLNH DYRGKDKPTNVLSFPFEAPPGVELDLLGDLIICRQVVEKEAEEQSKPLLAHWAH MVVHGSLHLLGYDHIEDDEAEEMESLETEIMQTMGFEDPYILEK;

SEQ ID NO: 42 is Aeromonas hydrophila subsp. hydrophila ATCC 7966 UPF0054 having the sequence MSVTLDLQLASASTDGLPSEAQLQGWLDGTILGFQQEAEVTVRIVDEAESNELN LTYRGKDKPTNVLSFPFEAPPGLELPLLGDLVICRQVVEREAAEQGKPLMAHWA HMVVHGSLHLLGYDHIEDDEAEEMETLERDIMQELGFADPYLNDEE;

SEQ ID NO: 43 is Yersinia pestis UPF0054 having the sequence MSQVILDLQIACADSQGLPTEGDFQRWLEAVLPLFQPVSEVTIRLVDEAESHDLN LTYRGKDKSTNVLSFPFEAPPEIELPLLGDLIICRQVVEKEAIEQEKALLAHWAH MVVHGSLHLLGYDHIDDEEAEEMELIETEIMHGLGYPDPYISEKDPD;

SEQ ID NO: 44 is Vibrio fischeri MJ11 UPF0054 having the sequence MSIELDLQIACENENGLPSEKDLMTWLNAVIPQFQPQAELTIRIVDEKESHELNHE YRGKDKPTNVLSFPFEAPPGLELDLLGDLIICRQVVEEEAIEQNKPLLAHWAHMV VHGSLHLLGYDHIEDDEAEEMESLETELMQGMGFEDPYIAEK;

SEQ ID NO: 45 is Haemophilus somnus 2336 UPF0054 having the sequence MKNVHDLQIASEDATNLPSVEQIQLWANAAIRAENSQPEMTVRIVDEEESHHLN LTYRGKDKPTNVLSFPFECPDEIELPLIGDLVICRQVVEREATEQEKPLMAHWAH to MIVHGSLHLLGYDHIEDDEAEEMERLETEIMLSLGFTDPYIIEK;

SEQ ID NO: 46 is Mannheimia succiniciproducens MBEL55E UPF0054 having the sequence MMKTVTIDLQIASEDQSNLPTLEQFTLWATNAVRAEHFEPEITIRIVDEAESHELN FTYRGKDRPTNVLSFPFECPEEVELPLLGDLVICRQVVEREAQEQGKPLTAHWA HMVVHGSLHLLGYDHIEDDEAVEMESLETEIMTGLGFEDPYSYDEE;

SEQ ID NO: 47 is Mannheimia haemolytica PHL213 UPF0054 having the sequence MENLYIDLQIACENTENLPSEQQFYTWVQKALAVEAKTDDFPESEITIRIVDEAES HELNLTYRGKDKPTNVLSFPFEAPEGIEMPLLGDLVICRQVMEKEAEEQGISAES HWAHLAIHGTLHLLGYDHIEEDEAVEMESLETEIMQSLGYEDPYLSEKI;

SEQ ID NO: 48 is Yersinia pseudotuberculosis YPIII UPF0054 having the sequence MSQVILDLQIACADSQGLPTEGDFQRWLEAVLPLFQPVSEVTIRLVDEAESHDLN LTYRGKDKSTNVLSFPFEAPPEIELPLLGDLIICRQVVEKEAIEQEKALLAHWAH MVVHGSLHLLGYDHIDDDEAEEMELIEIEIMHGLGYPDPYISEKDPD;

SEQ ID NO: 49 is Aggregatibacter actinomycetemcomitans UPF0054 having the sequence MGKMIIDLQIASADESGLPTAAQIEQWATAAVQPQSGEVEMTVRIVDETESHAL NFNYRGKDHPTNVLSFPFECPDEVELPLLGDLVICRQVVEREAQEQEKPLIAHW AHMVVHGSLHLLGYDHIEDDEAEEMESLETQIMMGLGFVDPYLSEK;

SEQ ID NO: 50 is Haemophilus influenzae UPF0054 having the sequence MGSVLVDLQIATENIEGLPTEEQIVQWATGAVQPEGNEVEMTVRIVDEAESHEL NLTYRGKDRPTNVLSFPFECPDEVELPLLGDLVICRQVVEREASEQEKPLMAHW AHMVVHGSLHLLGYDHIEDDEAEEMESLETQIMQGLGFDDPYLAEK;

SEQ ID NO: 51 is Actinobacillus succinogenes 130Z UPF0054 having the sequence MMKGVIIDLQIVSEDQTNLPTPEQFTQWATAAVRAEALEPEITIRIVDEAESHDLN LTYRGKDRPTNVLSFPFECPEEVELPLLGDLVICRQVVEREAEQQGKPLPAHWA HMVVHGCLHLLGYDHIEDDEAVEMESLETQIMTELGFEDPYSYDEI;

SEQ ID NO: 52 is Actinobacillus pleuropneumoniae L20 UPF0054 having the sequence MTTPIIDLQIAAENSENLPSLAQFTQWVQRALAHEAQTEDFPETEITIRIVDEAESY ELNLTYRGKDKPTNVLSFPFEVPEGIELPLLGDLIICRQVVEKEAQEQQISLESHW AHLAIHGTLHLLGYDHIEDAEAEEMEGLETEIMQSLGFEDPYISEKVIEE;

SEQ ID NO: 53 is Vibrio angustum S14 UPF0054 having the sequence MAIYLDLQHATESLDGLPTEAEFQQWLNAAVIPFQADAEVTIRLVDEKESHALN LEYRGKDRPTNVLSFPFEAPPGMELELLGDLIICRQVVEKEALEQNKPLKAHWA HMVVHGSLHLLGYDHIEDEEAEEMEGLETEIMQNMGFVDPYISEKQ;

SEQ ID NO: 54 is Vibrio shilonii AK1 UPF0054 having the sequence MAIELDLQIAVESEQGLPSADEFQGWLDKTITPFQQDAELTIRIVDEEESQQLNRD YRGKDKPTNVLSFPFEAPPGVEMDLLGDMIICRQVVEKEAIEQSKPLIAHWAHM VVHGSLHLLGYDHIEDDEAEEMEALETEIMLDMGFDDPYLVEKE;

SEQ ID NO: 55 is Moritella sp. PE36 UPF0054 having the sequence MNTTLDLQIATENEQKLPTEAQLNAWLNAVVKRFQDSAEVTIRIVDNTESQQLN NDYRGKDKPTNVLSFPFEVPEGIELDLLGDLIICKQVVEREAKEQQKPLTAHWA HMVIHGTLHLLGYDHIIDEEAEEMEGLETEIMLELEFEDPYITEK;

SEQ ID NO: 56 is Photobacterium sp. SKA34 UPF0054 having the sequence MAIYLDLQHATESLDGLPTEAEFQQWLNAAVIPFQADAEVTIRLVDEKESHTLN LEYRDKDRPTNVLSFPFEAPPGVELELLGDLIICRQVVEKEALEQNKPLKAHWA HMVVHGSLHLLGYDHIEDEEAEEMENLETEIMQNMGFVDPYISEKQ;

SEQ ID NO: 57 is Photobacterium profundum UPF0054 having the sequence MATYLDLQYATENQEGLPSEDKFQQWFEAAVTLFQSDAEITIRIVDVKESQALN LEYRGKDKPTNVLSFPFEAPPGIELDLLGDLIICRQVVEIEAKEQDKPLNAHWAH MVVHGTLHLLGYDHIEDEEAEEMEYLETEIIEKMGFADPFAAEKE;

SEQ ID NO: 58 is Haemophilus ducreyi UPF0054 having the sequence MNPIIDLQIASENSQGLPSAQQFSDWVKHALTYEAQTSNIPLTELTIRIVDEAESH HLNLTYRGKDKPTNVLSFPFEVPEGIELPLLGDLIICRQIVEKEALEQKISLDAHW AHLTIHGTLHLLGYDHIDEHEAEQMEGLESDIMQQLGFQDPYLAEK;

SEQ ID NO: 59 is Haemophilus parasuis 29755 UPF0054 having the sequence MNLYIDLQIASENTVGLPTAAQFQHWVDKALAMEAKTADYPETEITIRIVDEAES HELNLTYRGKDKPTNVLSFPFEMPEGIELPLLGDLIICRQVVEKEAVEQEKPLEA HWAHLAIHGTLHLLGYDHLTDEEAEEMESLETEIMQSLGFDDPYIAEKTIEE;

SEQ ID NO: 60 is Marinomonas sp. MWYL1 UPF0054 having the sequence MAYLELDLQIATEETANLPSEADFRLWVEKALPEVDEEFEVTIRIVDEEESHALN HEYRGKDKPTNVLSFPFEAPPGLELPLLGDLVICAQIVAKEAAEQNKELFHHWA HMTIHGILHLRGYDHINDDEADEMESIETELLASLSISDPYLIKE;

SEQ ID NO: 61 is Baumannia cicadellinicola str. Hc (Homalodisca coagulata) UPF0054 having the sequence MSKIILDLQVACDHRCNLPSEDLFMYWLYMVLPLFRKKAEVTIRLVDEAESYNL NKIYRGQNHSTNVLSFPFKAPSPVKLVLLGDIIICRQVVEREAQEQNKILEAYWA HMVIHGSLHLLGYDHFIEQNAKKMEYLETKIMHKLGYLNPYETEIS;

SEQ ID NO: 62 is Shewanella frigidimarina NCIMB 400 UPF0054 having the sequence MSQQGISLDLDLQIAVDNPRLPTQAEFETWVRAAIGQTKPVVELTIRIVDIAESQQ LNSTYRGKDKPTNVLSFPFEAPPEVELPLLGDLVICAPVVEQEAIEQNKPLIAHW AHMVIHGSLHLLGYDHIIDEEADEMESLETQLVEGLGFDNPYKEA;

SEQ ID NO: 63 is Shewanella sediminis HAW-EB3 UPF0054 having the sequence MNTSQAEIELDLDLQLAIENSQLPSKQNFELWVRTALPKTMAEAELTIRIVDEAE SQELNSTYRGKDKPTNVLSFPFEAPPEIEIPLLGDLIICAPVVELEALQQNKPLQAH WAHMVVHGCLHLLGYDHINDAEAEEMESLETQLVESLGFNNPYKEQ;

SEQ ID NO: 64 is Shewanella denitrificans OS217 UPF0054 having the sequence MSLSLDLDLQIAVDSNQLPSQADFETWVRTALGNTLDTAELTIRLVEIAESQSLN HDYRGKDKPTNVLSFPFEAPPGMELPLLGDLVICVAVVEQEALEQNKPLQAHW AHMVIHGCLHLLGYDHIIDQEAEEMESLETQLIEGLGFSNPYKEA;

SEQ ID NO: 65 is Shewanella pealeana ATCC 700345 UPF0054 having the sequence MSDSQMVIDLDVQVAVEGFELPSQAELELWVKTALRDTMSEAELTIRIVDVEES QELNMTYRGKDKPTNVLSFPFEAPPGIELPLLGDLVICAAVVEQEAIDQNKPLLA HWAHMVVHGCLHLLGYDHIEDVEAEEMESLETQLIESLGYINPYKEQ;

SEQ ID NO: 66 is Pseudoalteromonas atlantica T6c UPF0054 having the sequence MSAIVDLQVASDAANLPSADDFQRWLNAVLAHQQLSEHELTVRIVETQESQELN LTYRGKDKPTNVLSFPFEAPPGLSLNLLGDLVVCADVVAHEADEQHKKLHHHW AHMIVHGALHLLGFDHINDDEAKEMEALEVDILKQFSIDDPYQDQ

SEQ ID NO: 67 is Shewanella woodyi ATCC 51908 UPF0054 having the sequence MSTSHTKLELALDLQIATDNKQLPTQQDFELWVRTALRNTMTEAELTVRVVDA EESQALNSTYRGKDKPTNVLSFPFEAPAEIELPLLGDLIICASVVEHEAIQQNKPL QAHWAHMVVHGCLHLLGYDHINDIEAEEMESIETQLIESLGFNNPYKEQ;

SEQ ID NO: 68 is Shewanella halifaxensis HAW-EB4 UPF0054 having the sequence MNDNQTVIDLDLQIAVEGFELPSQAELELWVKTALRDTMSEAELTIRIVDVEESQ ELNSTYRGKDKPTNVLSFPFEAPPGIELPLLGDLVICAAVVKQEAMDQNKPLIAH WAHMVVHGCLHLLGYDHIDDSEAEEMESLETHLIESLGYINPYKEQ;

SEQ ID NO: 69 is Marinomonas sp. MED121 UPF0054 having the sequence MDNLDLDLQLASQDTKGLPSEADFMQWIQPALPQNDTCFELTVRIVDEEESQNL NHQYRGKDKSTNVLSFPFEVPDGIELPLLGDLVICRQVVEKEAVEQNKKLFHHW AHMVIHGTLHLRGFDHIKEDDAQEMESLEISILGQLDIPNPYLINE;

SEQ ID NO: 70 is Shewanella benthica KT99 UPF0054 having the sequence MSLNHNNLELDLDLQVATDNERLPSQEDFELWVRIALRDTMNQAELTIRIVDEA ESQTLNSSYRGKDKPTNVLSFPFEAPPEIDIPLLGDLIICAPVVEFEAKQQNKSLQS HWAHMVVHGCLHLLGYDHIQDAEAEEMESLETQLIESLGFNNPYKEQ;

SEQ ID NO: 71 is Reinekea sp. MED297 UPF0054 having the sequence MTELTLDLQIATDAENIPAEASFRQWVSAALSGYDREVEMSIRVVSAEESQDLN ATYRGKDKPTNVLSFPFEAPPGVADAGIDRLLGDLVICAPVVEEEAQAQRKHLS HHWAHMVVHGTLHLLGYDHIEDDEAEQMEQAERTILAALSIPDPYQGEREEPSE S;

SEQ ID NO: 72 is Shewanella loihica PV-4 UPF0054 having the sequence MMPDSPIALHLDLQVATQAEQLPSQEEFESWVRLALGNVMPEAEMTIRLVDEAE SQQLNHTYRGKDKPTNVLSFPFESPPEVELPLLGDLVICVPVVEQEAEIQGKSLTA HWAHMVVHGCLHLLGYDHIIDSEAEEMESLETQLIESLGFPNPYKEQ;

SEQ ID NO: 73 is Shewanella amazonensis SB2B UPF0054 having the sequence MALELALDLQFAVNPGNLPTEAEFETWVRVALGDTLDEAELTIRIVDATESQQL NRDYRGKDKPTNVLSFPFEAPPGMELPLLGDLVICASVVENEALEQHKALEAHW AHMVVHGCLHLLGYDHIDDAEAEEMEALETTLLTGLGYPDPYKEQ;

SEQ ID NO: 74 is Oceanobacter sp. RED65 UPF0054 having the sequence MIDVDVQIAIEGDNLPTPTQLQSWVTAALASLRPEAELSIRLVDNSESQSLNHEY RGKDKPTNVLSFPFEIPPELAEIENFTLIGDLVICHPVVCQEAAEQKKPLEHHYAH MVIHGCLHLLGYDHINEDEAEEMERLETDILATLNIPDPYIIN;

SEQ ID NO: 75 is Psychromonas ingrahamii 37 UPF0054 having the sequence MQLYVDLQIACSDPNDLPMPASFEKWIEAAILGGSESHREEAELTVRIVDQDEIM QLNHQYRNISKTTNVLAFPFQNPPGLTLPLLGDLIICKEVVESEAKLQGKSLTAH WAHMSIHSTLHLLGYDHIEQAEAVEMESLETKLLTELGFTDPYLSEKE;

SEQ ID NO: 76 is Pseudoalteromonas tunicata D2 UPF0054 having the sequence MTELDLQIASEFKDLPSFEQFQLWAHSTLDLFKEESELTIRIADEAESQQLNNDYR GKNKPTNVLSFPFEAPPGIELPLVGDLIICPQVVYQEALEQEKSFHDHFAHMVVH GCLHLLGFDHIEAEDAEEMESNEKEILAKLGISDPYRDDEE;

SEQ ID NO: 77 is Psychromonas sp. CNPT3 UPF0054 having the sequence MQLYIDLQIATMDEKALPSLALIETWIKEAIIKGSSITREEAELTVRIVDSVESQQL NGQYRHKDKPTNVLSFPFQAPMGIQLPLLGDLVICKQIVEQEALEQNKDLNAHW AHMLIHGSLHLLGFDHIIEQQALEMESLETKILVTLGFPPPYNETE;

SEQ ID NO: 78 is Marinobacter aquaeolei VT8 UPF0054 having the sequence MSKVTVDLQMAFDGTGVPSKTLFEAWAEAAWQGENPTEVTIRIVDNDESRELN HQYRGKDKPTNVLSFPFEAPAGITVPLAGDLVICAPVVEQEAREQNKDAVAHW AHMVVHGMLHLQGYDHIEVNEAEVMEALEIRLLAQLGFANPYEAEETEPDS;

SEQ ID NO: 79 is Neptuniibacter caesariensis UPF0054 having the sequence MSFDCDIQFEVEGNLPSESDFQRWVEAALSEFRDEGEICIRVVSPEESQELNHTYR DKDKPTNVLSFPFDIPEGIPIQLLGDLIICAEVVVTEASEQNKALFDHWAHMVVH GCLHLIGFDHIKDDEAEEMENLERQILASLDIADPYRDET;

SEQ ID NO: 80 is Saccharophagus degradans 2-40 UPF0054 having the sequence MLTIDIQQASTADASQLPSDKQFEIWVEAALQQRMNEAELSIRIVDEDESQALNL QYRGKDKSTNVLSFPCELPDGVELPLLGDLVICAQVVAKEALEQGKLLHAHWA HMVVHGTLHLLGYDHIEDGEAEEMEAIEIQVLLELGYPNPY;

SEQ ID NO: 81 is Candidatus Blochmannia pennsylvanicus str. BPEN UPF0054 UPF0054 having the sequence MTSNQVIIDLQLACKNLHGLPNRKMFQSWVSAIFSIYKKKIELTVRIVDIKEMHY LNWYYLKKDCPTNVLSFPFTPPLGMKSPLLGDVVLCRQIIEYESKEKNVPGRSH WAHMIIHGSLHLLGYNHIVDKEAILMQRVERNILQKCGYRTCCHVAHR;

SEQ ID NO: 82 is Shewanella sp. MR-4 UPF0054 having the sequence MSLDLALDVQYATASDYLPSEEQFALWVKTAIGNSMEQAELTIRIVDSRESQML NSTYRGKDKPTNVLSFPFEAPPEIELPLLGDLVICATVVENEAREQDKTLEAHWA HMVVHGCLHLLGYDHIEDEEAEEMESLETQLIESLGFTDPYKEQ;

SEQ ID NO: 83 is Shewanella sp. ANA-3 UPF0054 having the sequence MSLDLALDVQYATASDYLPSEDQFALWVKTAIGNSMEQAELTIRIVDARESQML NSTYRGKDKPTNVLSFPFEAPPEIELPLLGDLVICAAVVENEAREQDKTLEAHWA HMVVHGCLHLLGYDHIEDEEAEEMESLETQLIESLGFTDPYKEQ;

SEQ ID NO: 84 is Homo sapiens UPF0054 having the sequence MSLVIRNLQRVIPIRRAPLRSKIEIVRRILGVQKFDLGIICVDNKNIQHINRIYRDRN VPT DVLSFPFHEHLKAGEFPQPDFPDDYNLGDIFLGVEYIFHQCKENEDYNDVLTVTA TG LCHLLGFTHGTEAEWQQMFQKEKAVLDELGRRTGTRLQPLTRGLFGGS;

SEQ ID NO: 85 is Rattus norvegicus UPF0054 having the sequence MSLVIKNLQRVVPIRRMPLRRKMDLVRSILGVKKFDLGIICVDNKNIQNINRIYR NKNVPTDVLSFPFHENLKAGEFPQPRSPDDYNLGDVFLGVEYIFQYCKGNEDYY DVLTVTATHGLCHLLGFTHSSEAEWQKMYNQEKLVLEELSRHTGARLQPLSRG LY;

SEQ ID NO: 86 is Mus musculus UPF0054 having the sequence MSLVIKNLQRVVPIRRVPLRRKMDLVRSILGVKKFDLGIICVDNKTIQNINRIYRN KNVPTDVLSFSFHENLKAGEFPQPHSPDDYNLGDIFLGVEYILQHCRESEDYCDV LTVTATHGLCHLLGFTHSSKAEWQKMYNQEKLVLEELSRYTGARLQPLSRGLY;

SEQ ID NO: 87 is Ornithorhynchus anatinus UPF0054 having the sequence MSLVLRNLQRVIPIRRVPLRQKIETLRRILGVRRFDLGIVCVDNKNIQRVNNTYR QKNVPTDVLSFPFHENLKAGEMPQPRFPEDYNLGDVFLGVEYIFQQCQEGNEDF YGVLTVIAAHGLCHLLGYRHDTEAEWQEMYQKEKHILDELNKLTGTNLQPLTK NHF;

SEQ ID NO: 88 is Bos taurus UPF0054 having the sequence MSLALRNLQRAVPLRRALLRQRLQAVRGALGVRAFGLGVVCVDNRKIQQMNRI YREQDTPTDVLSFPFHENLKAGEIPQPDFPDDYNLGDIFLGVEFIFQQCKEDEDY YDILTVTATHGLCHLLGFTHSTEAEWQKMYQKEKQVLQELNKHMGTRLQPLSR GLF;

SEQ ID NO: 89 is Monodelphis domestica UPF0054 having the sequence MSLLLRNLQSAIPLRRAPLRARIELLRHILGIRRFDLGVVCVDNQGIQRLNRIYRQ AHGPTDVLSFPFHEDLRAGAVPQPECPDDLNLGDIFLGVEYIFHQCQENGENYY DILTVTAAHGLCHLLGYKHNTAAEWREMFQKEKLTLEELNRVAGTSLQPLTKN LFG;

SEQ ID NO: 90 is Canis lupus familiaris UPF0054 having the sequence MSLVLRVPQRAVPVRRAPLRSRVELLRAVLGVRDFDLGLLCVDNEGMQRLNRA YRGDDRPTDVLSFPFHENVKAGELPRPRSRDDYNLGDIVLGVEYVFQRCRGDAD YYDALTVTAAHGLCHLLGFTHSTEAEWRKMYQKEKQVLEELSRLTGTRLQPLS RGLF;

SEQ ID NO: 91 is Pan troglodytes UPF0054 having the sequence MWRPGRAELRGGRKRRLWRRPSPHAHRLAPGPRGPRCPLAPRKDLRRKHRLLP RHSALRTAGRKRRGPTSGSLSTTGFRPARCDPVPLPPTRSVAGLPVGRVRPLSRP LLGPDTGSVANIFKGLVILREMSLVIRNLQRVIPIRRAPLRSKIEIHLKAGEFPQPD FPDDYNLGDIFLGVEYIFHQCKENEDYNDILTVTATHGLCHLLGFTHGTEAEWQ QMFQKEKAVLDELGRRTGTRLQPLTRGLFGGS;

SEQ ID NO: 92 is Danio rerio UPF0054 having the sequence MGVIVRNLQNVVPVRRARLRRDVEILRHIFGVQKFDMGIICVDNRKIQRINHTYR RRNQPTDVLSFPFYEDLRPGKVPCALQRDEYNLGDIFLGVEYVMQQCKETKQDL HQTLTVVTAHGICHLLGYRHETEEEWNEMQQKESYILSEFNRLTGSHLEPLTKR;

SEQ ID NO: 93 is Xenopus laevis UPF0054 having the sequence MSLILRNAQHAVPLYRAHLRFSLDIARSCLKVKNFDLGIICVNNARIQHINRVYR GQDSVTDVLSFPFHEDLNPSLLPIPATPDEYNLGDIYLGVAFIYEQCQKTQEDYRS ILTITAVHGLCHLLGHKHNNPEKWKQMFEKETEILNEINRVTGSKLKPLSTNHY;

SEQ ID NO: 94 is Strongylocentrotus purpuratus UPF0054 having the sequence MSIIVRNFQKSVAFNLDKVKGDLKALRRILRVERFDVSVICVEDKDIRQMNKIYR ATDEPTDVLSFPAHENLKPGRLPDPWSDLADLGDMFLGMGYIERDCTKHNADID DVLPVIITHGLCHLIGYDHKTQEQWKMMHDRELQILEQFKKLTGQDLQPLTGRS DFLQAHHTDPQPHSQPTPA;

SEQ ID NO: 95 is Nematostella vectensis UPF0054 having the sequence MSVFVRNFQQRVLFSEALLERDARVLVQLLKADRFDVSIVCAGGKRIKSLNLKY RRRNVQTDVLAFPYFENMKPGVLPNPKLQDDWNLGDVILGMPVIHQDCQEDNK SVQEYLPVLITHGLCHLLGYTHDTEQNLQQMHKKEKEVLSGFNQVTGYNTKPL LPAITPSDR; and

SEQ ID NO: 96 is UPF0054 conserved histidine triad motif having the sequence HXXXHXXXXXH, where H is a histidine residue and X is any amino acid.

### DETAILED DESCRIPTION

The present invention generally relates to various nucleases and uses thereof, and in some cases, to the UPF0054 protein superfamily. Members of the UPF0054 protein superfamily, such as the E. coli protein YbeY, may possess RNase activity and may be involved in certain important cellular processes. Disruption of YbeY activity can lead to increased sensitivity to antibiotics. Accordingly, certain embodiments of the invention are directed to systems and methods for screening target compounds for activity against UPF0054 superfamily proteins. In some embodiments, the screening method allows target compositions to be determined that show selective activity against UPF0054 superfamily proteins. Other embodiments of the invention provide for nucleases capable of site-specific cleavage of nucleic acids. Still other embodiments are directed to inhibitors or methods for inhibiting cells (e.g., by disrupting UPF0054 protein superfamily activity), methods of treating subjects using such inhibitors, optionally with other antibiotics, and the like.

“UPF0054” refers to a superfamily of proteins commonly defined by sequence similarity, as discussed herein. Examples of such UPF0054 proteins include, but are not limited to, the E. coli homolog YbeY and the human homolog C21orf57, as well as SEQ ID NO. 1-95. “UPF” is an abbreviation for “Uncharacterized Protein Families.” Proteins belonging to the UPF0054 superfamily have a conserved histidine triad region within the protein having the sequence HXXXHXXXXXH (SEQ ID NO: 96), where H is a histidine residue and X is any suitable amino acid. Typically, a UPF0054 protein will have a molecular weight of about 17 kDa to about 21 kDa.

A UPF0054 protein will generally exhibit nuclease activity and will cleave random RNA or other polynucleotide sequences, e.g., the UPF0054 protein can hydrolyze or cleave a bond within a polynucleotide sequence. For instance, the nuclease activity of a UPF0054 protein may be at least 250 Units per mg of isolated protein. As used herein, a “Unit” is defined as 1 microgram of randomly-structured single stranded messenger RNA each having a length of about 500 nucleotides cleaved per minute by the enzyme at 37° C. and in a 20 microliter solution at pH 7.5 comprising 50 mM HEPES, 1 microgram RNA, and 4 micrograms YbeY protein. In this context, the cleavage may be single (e.g., a polynucleotide is divided into two polynucleotides), or in some cases, multiple cleavages of the same polynucleotide can occur. In some instances, the nuclease activity may be at least about 100 Units per mg, at least about 10 Units per mg, at least about 1 Unit per mg, at least about 0.1 Units per mg, at least about 0.01 Units per mg, etc. In other instances, the nuclease activity may be between about 0.001 Units per mg and 0.1 Units per mg, about 0.01 Units per mg and 1 Unit per mg, about 0.1 Units per mg and 10 Units per mg, about 1 Unit per mg and 100 Units per mg, about 10 Units per mg and 250 Units per mg, about 100 Units per mg and 500 Units per mg, etc. In some instances, the nuclease activity may be less than about 500 Units per mg, less than about 250 Units per mg, less than about 100 Units per mg, less than about 10 Units per mg, less than about 1 Units per mg, less than about 0.1 Units per mg, less than about 0.01 Units per mg, less than about 0.001 Units per mg, etc.

One method of identifying a protein as a UPF0054 protein using a simple screening test is as follows. Using the PROSITE methodology (Sigrist C. J. A. et al. PROSITE: a documented database using patterns and profiles as motif descriptors. 2002, Brief Bioinform. 3:265-274, incorporated herein by reference), in general, a candidate protein amino acid sequence is scanned against a database of known biologically significant sites, and patterns within protein amino acid sequences are compared using statistical methods, such as the ones described in the Sigrist reference, to the candidate protein amino acid sequence in order to identify the known family of protein (if any) to which the candidate protein belongs. An example database can be found in Hulo N., et al. The PROSITE database. 2006, Nucleic Acid Res. 34:D227-D230, incorporated herein by reference. Proteins belonging to the UPF0054 protein superfamily can then be identified by their matches against other proteins of the UPF0054 protein superfamily, as this superfamily is defined within databases such as to those described in the Hulo reference.

It should also be understood that a protein may be identified as being within the UPF0054 family, as defined herein, even if it has been modified in some way, for example through mutation, chemical modification, truncation, fusion with another protein, etc., so long as the protein can still be identified as discussed above. Other examples of modifications include posttranslational modifications; for example, a UPF0054 superfamily protein may be glycosylated, acylated, methylated, phosphorylated, lipoylated, etc.

UPF0054 superfamily proteins may be found in a wide variety of organisms including bacteria and some eukaryotic organisms, such as those described herein. Members of the UPF0054 protein superfamily appear to be nearly ubiquitous among bacteria. In addition, the UPF0054 protein superfamily appears to be one that has been relatively highly conserved between species. For instance, an UPF0054 ortholog taken from a first species and inserted into a second different species having a disabled UPF0054 protein appears to function in a similar manner in the second species, for example, “rescuing” the second species in terms of function of the UPF0054 protein within the second species. Accordingly, in various embodiments of the invention, as discussed below, a UPF0054 protein from a first species can be used in conjunction with a second species with similar functionality.

As mentioned, UPF0054 proteins are found in a large range of organisms, as shown in the sequences described above, including bacteria and eukaryotes, such as humans. In addition, certain aspects of the invention are directed to compositions including such UPF0054 proteins, including isolated proteins. As used herein, an “isolated” molecule refers to a molecule that is free of other substances with which it is ordinarily found in nature or in vivo systems to an extent practical and appropriate for its intended use. In particular, the isolated molecule is sufficiently free from other biological constituents of host cells or if they are expressed in host cells they are free of the form or context in which they are ordinarily found in nature. For instance, a UPF0054 protein may be isolated from an organism. Alternatively, an isolated UPF0054 protein may be removed from the host cell or present in a host cell that does not ordinarily express such a protein. Note that the protein may be admixed with other compounds, for example, buffers, excipients, stabilizers, or the like. The protein is nonetheless isolated in that it has been substantially separated from the substances with which it may be associated in living systems.

In one embodiment, UPF0054 proteins can be isolated from extremophilic organisms, for example from thermophiles such as Thermus thermophilus, which may have greater stability at, below, and/or above 37° C. relative to UPF0054 proteins isolated from non-extremophilic organisms. UPF0054 proteins isolated from extremophiles may have catalytic activity at temperatures much higher than 37° C., e.g. above about 60° C., above about 80° C., or above about 100° C. UPF0054 proteins isolated from psychrophiles, such as Colwellia psychrerythraea may have activity at temperatures much lower than 37° C., for example, below about 15° C., below about 10° C., or below about 5° C. Other extremophiles are known in the art and may have UPF0054 proteins with greater stability and/or activity under certain conditions in comparison to UPF0054 proteins isolated from mesophilic organisms. Accordingly, certain embodiments of the invention are directed to uses of UPF0054 proteins able to function under such conditions. For instance, a UPF0054 protein (which may be isolated) may exhibit catalytic activity at relatively high or relatively low temperatures, such as those described above.

Some aspects of the invention comprise modified UPF0054 proteins. In some embodiments, for example, fusion proteins comprising a UPF0054 protein may be used. For instance, the fusion protein can contain a tag attached to a UPF0054 protein, which in some instances can be used to purify the protein. Examples of purification tags include poly(His), FLAG, chitin binding protein (CBP), maltose binding protein (MBP), glutathione-s-transferase (GST), etc. Other fusion proteins that may be produced include those comprising solubility tags, e.g. thioredoxin; fluorescence tags, e.g. GFP and related fluorescent proteins; antibodies; enzymes, e.g. horseradish peroxidase; targeting sequences, e.g. sequences that direct proteins to the mitochondria, a membrane, export from the cell; etc. It should be understood that tags may be classified under one category but may find utility in other categories. For instance, MBP may be used as a solubility tag instead of or in addition to being used as a purification tag.

As discussed, proteins of the UPF0054 superfamily display nuclease activity, and can be used to cleave polynucleotides such as RNA. Accordingly, one aspect of the invention provides for a composition comprising a protein of the UPF0054 superfamily and at least one polynucleotide. A “polynucleotide,” as used herein, comprises at least two nucleotides linked together by a phosphate group, each with a nucleobase and a sugar. The 5′ and 3′ end of the polynucleotide may or may not be phosphorylated or otherwise modified. Polynucleotides include at least one DNA or RNA in any form, such as genomic DNA, cDNA, mRNA, rRNA, tRNA, siRNA, miRNA, etc. In some cases, the polynucleotide may be an oligomer of DNA and/or RNA.

Polynucleotides may also contain one or more modified nucleotides. Modified nucleotides may be naturally occurring nucleotides other than those nucleotides containing guanine, adenine, cytosine, thymine, and uracil. For example, the modified nucleotide may contain a methylated nucleobase. The polynucleotide could originate from in vivo sources, i.e. a cell or tissue, in vitro sources such as nucleic acid amplification methods, i.e. polymerase chain reaction (PCR), or may be produced synthetically by well known automated and non-automated techniques. The polynucleotide can be single-stranded or double-stranded or a combination of single-stranded and double-stranded regions. In some instances, the polynucleotide may adopt a secondary structure through, for example, self-hybridization.

In one embodiment, the polynucleotide is a 16S rRNA precursor, such as a 17S rRNA. The 16S rRNA is the RNA component of the 30S subunit of a prokaryotic ribosome, which binds the Shine-Dalgarno sequence of an mRNA molecule and is well-known to those of ordinary skill in the art. Without wishing to be bound by any theory, it is believed that the proteins of the UPF0054 superfamily are involved in the maturation of 16S rRNA from its precursors. As the 16S rRNA precursors are typically longer than the mature 16S rRNA, it is further believed that the UPF0054 protein acts on the 16S rRNA precursor by forming a complex with the precursor, and cleaving a portion of the precursor. It is also believed that the UPF0054 protein can act on a polynucleotide by recognizing motifs such as those described below; however, in some cases, e.g., if the polynucleotide does not contain such a motif, the UPF0054 protein may still act on the polynucleotide, e.g., at a random location within the polynucleotide. In eukaryotes, the UPF0054 proteins appear to play a similar role. The UPF0054 proteins may act on mitochondrial RNA, such as tRNA, which may help in the maturation of such RNAs within eukaryotic cells. In addition, eukaryotic UPF0054 proteins, when exposed to 16S rRNA precursors, can cause cleavage of the precursors and can accordingly be used to facilitate maturation of the precursor into the final 16S rRNA.

In some embodiments, the UPF0054 protein acts on a single-stranded polynucleotide. In these instances, the polynucleotide may be cleaved at one or more locations in the polynucleotide, and in some cases, the cleavage locations are random. In other embodiments, however, the UPF0054 protein may act on a double-stranded region of the polynucleotide, as discussed below. It should be noted that the entire polynucleotide need not be double-stranded; for instance, in one embodiment, the polynucleotide may be single stranded but contain a double-stranded region through, for example, self-hybridization. In certain embodiments, the UPF0054 protein acts on helices of duplexes of polynucelotides, e.g., duplexes of DNA and/or RNA. These may be present as single polynucleotides or more than one nucleotide within the helix or duplex. For instance, the nucleotide may include helices of DNA and/or RNA, optionally with overhangs such as a 3′ overhang and/or a 5′ overhang. In another embodiment, the polynucleotide may form a hairpin structure.

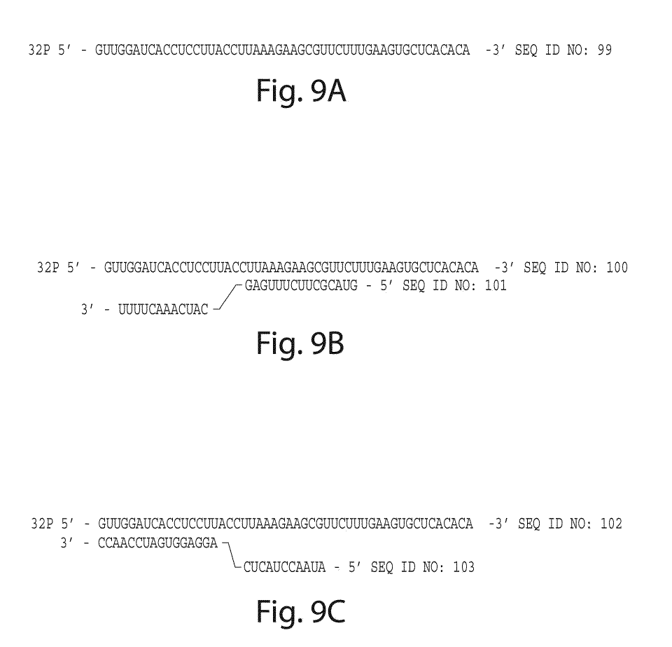

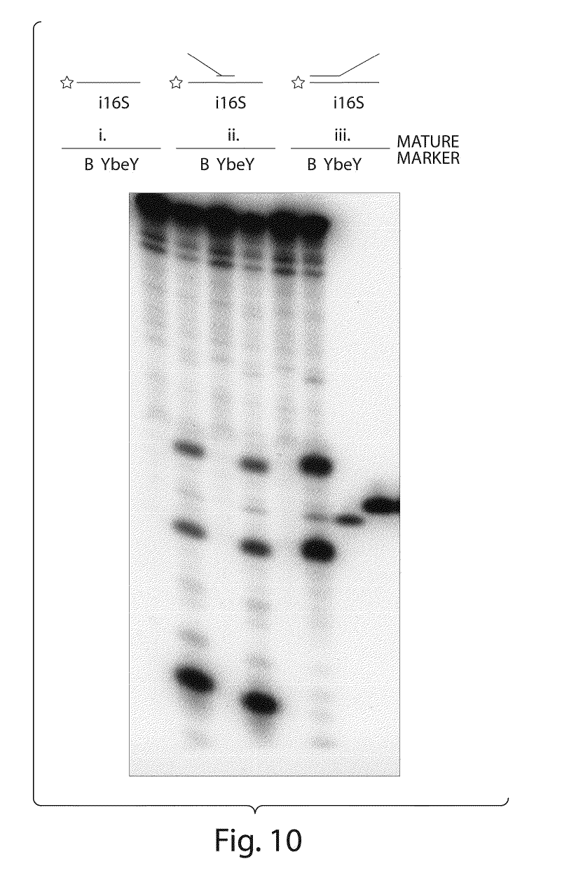

In certain embodiments, cleavage occurs site-specifically within a polynucleotide, i.e. at a specific location within the polynucleotide sequence. For example, cleavage of the polynucleotide may occur specifically at a point where a branch occurs in the polynucleotide. In other embodiments, however, cleavage may occur at other locations in addition to cleavage at a branch point. Cleavage can also occur at locations other than at a branch point. A branch point or a “flap” is the point at which two polynucleotides diverge from a hybridized double-stranded structure to two single-stranded structures, for example as illustrated in FIG. 9B and FIG. 9C. A branch point could also be generated by the secondary structure of a single polynucleotide.

Accordingly, the UPF0054 protein is able to act on virtually any RNA molecule, besides just 16S rRNA precursors. Thus, certain embodiments are directed to isolated UPF0054 proteins, and complexes of UPF0054 proteins that have been bound to suitable polynucleotides. In some cases, the UPF0054 protein may cleave the polynucleotide at specific sites (e.g., if the polynucleotide displays branch points), such as those described above; in other cases, however, the UPF0054 protein may cleave the polynucleotide at random locations within the polynucleotide.

In the above embodiments, the polynucleotide may be RNA; however the polynucleotide may also comprise other nucleic acids as discussed above. As examples, the polynucleotide may comprise RNA containing modified nucleotides such as 2′-fluoronucleotides, or the polynucleotide may contain more than one type of nucleic acid, e.g., DNA and RNA. In another example, the polynucleotide may be resistant to cleavage, e.g. the polynucleotide may contain a phosphothioate group in place of a phosphate group linking two nucleotides together. Use of a UPF0054 protein for cleaving a polynucleotide and/or for any other application may occur in aqueous solution, and the solution may include various buffers, salts, adjuncts, stabilizers, etc. known to those skilled in the art.

The binding affinity of a polynucleotide for a UPF0054 protein can be determined using any of a number of techniques known to those of ordinary skill in the art. A non-limiting example of such an assay is a gel shift assay. Gel shift assays are known to those skilled in the art and can be performed by exposing a labeled polynucleotide (i.e. radiolabeled, fluorescently labeled, etc.) at a fixed concentration to a UPF0054 protein in a plurality of solutions containing the UPF0054 protein at a plurality of concentrations. The binding affinity can be determined, for instance, by non-denaturing gel electrophoresis, in which unbound polynucleotide migrates through the gel at a different rate than the polynucleotide-protein complex, thus allowing the polynucleotide-protein complex to be separated from unbound polynucleotide. The amount of polynucleotide-protein complex formed in the solution in this example assay may be indicative of the concentration of protein. The amount of polynucleotide-protein complex relative to unbound polynucleotide can also be quantified in some cases by known methods, for example phosphorimagery if the polynucleotide is radiolabeled. From these data, the affinity of the polynucleotide for the protein can be determined. Since UPF0054 proteins are nucleases, in some cases, inhibition of the nuclease activity may also be performed so that the polynucleotide remains intact during the assay. As disclosed herein, 5 mM Mg²⁺ or EDTA can be used to inhibit the nuclease activity of the UPF0054 protein.

In another aspect of the invention, a nucleotide sequence such as one encoding UPF0054 is delivered into a cell. Any method or delivery system may be used for the delivery and/or transfection of the nucleic acid in the cell, for example, but not limited to particle gun technology, colloidal dispersion systems, electroporation, vectors, and the like.

In its broadest sense, a “delivery system,” as used herein, is any vehicle capable of facilitating delivery of a nucleic acid (or nucleic acid complex) to a cell and/or uptake of the nucleic acid by the cell. Other example delivery systems that can be used to facilitate uptake by a cell of the nucleic acid include calcium phosphate and other chemical mediators of intracellular transport, microinjection compositions, and homologous recombination compositions (e.g., for integrating a gene into a preselected location within the chromosome of the cell).

The term “transfection,” as used herein, refers to the introduction of a nucleic acid into a cell. Transfection may be accomplished by a variety of means known to the art. Such methods include, but are not limited to, particle bombardment mediated transformation (e.g., Finer et al., Curr. Top. Microbiol. Immunol., 240:59 (1999)), viral infection (e.g., Porta and Lomonossoff, Mol. Biotechnol. 5:209 (1996)), microinjection, electroporation, and liposome-mediated delivery. Standard molecular biology techniques are common in the art (See e.g., Sambrook, J. et al., Molecular Cloning: A Laboratory Manual, 2^{nd} ed., Cold Spring Harbor Laboratory Press, New York (1989)).

For instance, in one set of embodiments, genetic material may be introduced into a cell using particle gun technology, also called microprojectile or microparticle bombardment, which involves the use of high velocity accelerated particles. In this method, small, high-density particles (microprojectiles) are accelerated to high velocity in conjunction with a larger, powder-fired macroprojectile in a particle gun apparatus. The microprojectiles have sufficient momentum to penetrate cell walls and membranes, and can carry DNA or other nucleic acids into the interiors of bombarded cells. It has been demonstrated that such microprojectiles can enter cells without causing death of the cells, and that they can effectively deliver foreign genetic material into intact tissue.

In another set of embodiments, a colloidal dispersion system may be used to facilitate delivery of the nucleic acid (or nucleic acid complex) into the cell. As used herein, a “colloidal dispersion system” refers to a natural or synthetic molecule, other than those derived from bacteriological or viral sources, capable of delivering to and releasing the nucleic acid to the cell. Colloidal dispersion systems include, but are not limited to, macromolecular complexes, beads, and lipid-based systems including oil-in-water emulsions, micelles, mixed micelles, and liposomes. One example of a colloidal dispersion system is a liposome. Liposomes are artificial membrane vessels. It has been shown that large unilamellar vessels (“LUV”), which range in size from 0.2 to 4.0 microns can encapsulate large macromolecules within the aqueous interior and these macromolecules can be delivered to cells in a biologically active form (Fraley, et al., Trends Biochem. Sci., 6:77 (1981)).

Lipid formulations for transfection and/or intracellular delivery of nucleic acids are commercially available, for instance, from QIAGEN, for example as EFFECTENE® (a non-liposomal lipid with a special DNA condensing enhancer) and SUPER-FECT® (a novel acting dendrimeric technology) as well as Gibco BRL, for example, as LIPOFECTIN® and LIPOFECTACE®, which are formed of cationic lipids such as N-[1-(2,3-dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes were described in a review article by Gregoriadis, G., Trends in Biotechnology 3:235-241 (1985).

Electroporation may be used, in another set of embodiments, to deliver a nucleic acid (or nucleic acid complex) to the cell. Electroporation, as used herein, is the application of electricity to a cell in such a way as to cause delivery of the nucleic acid into the cell without killing the cell. Typically, electroporation includes the application of one or more electrical voltage “pulses” having relatively short durations (usually less than 1 second, and often on the scale of milliseconds or microseconds) to a media containing the cells. The electrical pulses typically facilitate the non-lethal transport of extracellular nucleic acids into the cells. The exact electroporation protocols (such as the number of pulses, duration of pulses, pulse waveforms, etc.), will depend on factors such as the cell type, the cell media, the number of cells, the substance(s) to be delivered, etc., and can be determined by one of ordinary skill in the art.

In yet another set of embodiments, the nucleic acid may be delivered to the cell in a vector. In its broadest sense, a “vector” is any vehicle capable of facilitating the transfer of the nucleic acid to the cell such that the nucleic acid can be processed and/or expressed in the cell. Preferably, the vector transports the nucleic acid to the cells with reduced degradation, relative to the extent of degradation that would result in the absence of the vector. The vector optionally includes gene expression sequences or other components able to enhance expression of the nucleic acid within the cell. The invention also encompasses the cells transfected with these vectors. Examples of such cells have been previously described.

In general, vectors useful in the invention include, but are not limited to, plasmids, phagemids, viruses, other vehicles derived from viral or bacterial sources that have been manipulated by the insertion or incorporation of the nucleotide sequence (or precursor nucleic acid) of the invention. Viral vectors useful in certain embodiments include, but are not limited to, nucleic acid sequences from the following viruses: retroviruses such as Moloney murine leukemia viruses, Harvey murine sarcoma viruses, murine mammary tumor viruses, and Rous sarcoma viruses; adenovirus, or other adeno-associated viruses; SV40-type viruses; polyoma viruses; Epstein-Barr viruses; papilloma viruses; herpes virus; vaccinia virus; polio viruses; and RNA viruses such as retroviruses. One can readily employ other vectors not named but known to the art.

Some viral vectors can be based on non-cytopathic eukaryotic viruses in which non-essential genes have been replaced with the nucleotide sequence of interest. Non-cytopathic viruses include retroviruses, the life cycle of which involves reverse transcription of genomic viral RNA into DNA with subsequent proviral integration into host cellular DNA.

Genetically altered retroviral expression vectors may have general utility for the high-efficiency transduction of nucleic acids. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the cells with viral particles) can be found in Kriegler, M., Gene Transfer and Expression, A Laboratory Manual, W.H. Freeman Co., New York (1990) and Murry, E. J. Ed., Methods in Molecular Biology, Vol. 7, Humana Press, Inc., Cliffton, N.J. (1991), both hereby incorporated by reference.

Another example of a virus for certain applications is the adeno-associated virus, which is a double-stranded DNA virus. The adeno-associated virus can be engineered to be replication-deficient and is capable of infecting a wide range of cell types and species. The adeno-associated virus further has advantages, such as heat and lipid solvent stability; high transduction frequencies in cells of diverse lineages, including hemopoietic cells; and/or lack of superinfection inhibition, which may allow multiple series of transductions.

Another vector suitable for use with the invention is a plasmid vector. Plasmid vectors have been extensively described in the art and are well-known to those of skill in the art. See e.g., Sambrook, et al., Molecular Cloning: A Laboratory Manual, Second Edition, Cold Spring Harbor Laboratory Press, 1989. These plasmids may have a promoter compatible with the host cell, and the plasmids can express a polypeptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom-designed, for example, using restriction enzymes and ligation reactions, to remove and add specific fragments of DNA or other nucleic acids, as necessary. The present invention also includes vectors for producing nucleic acids or precursor nucleic acids containing a desired nucleotide sequence. These vectors may include a sequence encoding a nucleic acid and an in vivo expression element, as further described below. In some cases, the in vivo expression element includes at least one promoter.

The nucleic acid, in one embodiment, may be operably linked to a gene expression sequence which directs the expression of the nucleic acid within the cell. The nucleic acid sequence and the gene expression sequence are said to be “operably linked” when they are covalently linked in such a way as to place the transcription of the nucleic acid sequence under the influence or control of the gene expression sequence. A “gene expression sequence,” as used herein, is any regulatory nucleotide sequence, such as a promoter sequence or promoter-enhancer combination, which facilitates the efficient transcription and translation of the nucleotide sequence to which it is operably linked. The gene expression sequence may, for example, be a eukaryotic promoter or a viral promoter, such as a constitutive or inducible promoter. Promoters and enhancers consist of short arrays of DNA sequences that interact specifically with cellular proteins involved in transcription, for instance, as discussed in Maniatis, T. et al., Science 236:1237 (1987), incorporated herein by reference. Promoter and enhancer elements have been isolated from a variety of eukaryotic sources including genes in plant, yeast, insect and mammalian cells and viruses (analogous control elements, i.e., promoters, are also found in prokaryotes).

The selection of a particular promoter and enhancer depends on what cell type is to be used and the mode of delivery. For example, a wide variety of promoters have been isolated from plants and animals, which are functional not only in the cellular source of the promoter, but also in numerous other plant and/or animal species. There are also other promoters (e.g., viral and Ti-plasmid) which can be used. For example, these promoters include promoters from the Ti-plasmid, such as the octopine synthase promoter, the nopaline synthase promoter, the mannopine synthase promoter, and promoters from other open reading frames in the T-DNA, such as ORF7, etc. Promoters isolated from plant viruses include the 35S promoter from cauliflower mosaic virus (CaMV). Promoters that have been isolated and reported for use in plants include ribulose-1,3-biphosphate carboxylase small subunit promoter, phaseolin promoter, etc.

Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

Thus, a variety of promoters and regulatory elements may be used in the expression vectors of the present invention. For example, in some preferred embodiments an inducible promoter is used to allow control of nucleic acid expression through the presentation of external stimuli (e.g., environmentally inducible promoters). Thus, the timing and amount of nucleic acid expression may be controlled. Non-limiting examples of expression systems, promoters, inducible promoters, environmentally inducible promoters, and enhancers are described in International Patent Application Publications WO 00/12714, WO 00/11175, WO 00/12713, WO 00/03012, WO 00/03017, WO 00/01832, WO 99/50428, WO 99/46976 and U.S. Pat. Nos. 6,028,250, 5,959,176, 5,907,086, 5,898,096, 5,824,857, 5,744,334, 5,689,044, and 5,612,472.

As used herein, an “expression element” can be any regulatory nucleotide sequence, such as a promoter sequence or promoter-enhancer combination, which facilitates the efficient expression of the nucleic acid. The expression element may, for example, be a mammalian or viral promoter, such as a constitutive or inducible promoter. Constitutive mammalian promoters include, but are not limited to, polymerase promoters as well as the promoters for the following genes: hypoxanthine phosphoribosyl transferase (HPTR), adenosine deaminase, pyruvate kinase, and alpha-actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. Promoters useful as expression elements of the invention also include inducible promoters. Inducible promoters are expressed in the presence of an inducing agent. For example, a metallothionein promoter can be induced to promote transcription in the presence of certain metal ions. Other inducible promoters are known to those of ordinary skill in the art. The in vivo expression element can include, as necessary, 5′ non-transcribing and 5′ non-translating sequences involved with the initiation of transcription, and can optionally include enhancer sequences or upstream activator sequences.

In another set of embodiments, homologous recombination can be used to alter the expression of UPF0054 proteins. In some instances, recombination can be used to alter a promoter of UPF0054 expression. In other instances, the UPF0054 gene itself can be altered or deleted. In some embodiments, the promoter for a UPF0054 protein can be used to monitor the expression of the UPF0054 protein, for example by using the promoter for a UPF0054 protein to drive the expression of an indicator such as a fluorescent protein.

Using any gene transfer technique, such as the above-listed techniques, an expression vector harboring the nucleic acid may be transformed into a cell to achieve temporary or prolonged expression. Any suitable expression system may be used, so long as it is capable of undergoing transformation and expressing of the precursor nucleic acid in the cell. In one embodiment, a pET vector (Novagen, Madison, Wis.), or a pBI vector (Clontech, Palo Alto, Calif.) is used as the expression vector. In some embodiments an expression vector further encoding a green fluorescent protein (GFP) is used to allow simple selection of transfected cells and to monitor expression levels. Non-limiting examples of such vectors include Clontech's “Living Colors Vectors” pEYFP and pEYFP-C1.

In some cases, a selectable marker may be included with the nucleic acid being delivered. As used herein, the term “selectable marker” refers to the use of a gene that encodes an enzymatic or other detectable activity (e.g., luminescence or fluorescence) that confers the ability to grow in medium lacking what would otherwise be an essential nutrient. A selectable marker may also confer resistance to an antibiotic or drug upon the cell in which the selectable marker is expressed. Selectable markers may be “dominant” in some cases; a dominant selectable marker encodes an enzymatic or other activity (e.g., luminescence or fluorescence) that can be detected in any cell or cell line.

In some embodiments, magnesium(II) can inhibit a UPF0054 protein. Thus, the present invention discloses, in another aspect, compositions comprising UPF0054 proteins in the presence of magnesium(II) at concentrations less than about 5 mM, less than about 2 mM, less than about 1 mM, less than about 0.5 mM, less than about 0.05 mM, or less than about 0.001 mM. In some instances, the ability of magnesium(II) to inhibit UPF0054 proteins may be used to inhibit the enzyme deliberately, for example in a biochemical assay. In other instances, the enzyme may be inhibited by a chelating agent, such as EDTA (ethylenediaminetetraacetic acid). Various stabilizers and adjuncts known in the art can be used to preserve the activity of the purified protein. For instance, compositions comprising a UPF0054 protein and buffer, salts, detergents, glycerol, BSA (bovine serum albumin), DTT (dithiothreitol), 2-mercaptoethanol, etc. are contemplated. Other ions that may inhibit UPF0054 protein, in some (but not all) instances, include Mn²⁺, Zn²⁺, Co²⁺, or Ca²⁺.

In other embodiments, a UPF0054 protein is kept in a solution containing magnesium(II) at a concentration sufficient to reduce the activity of the UPF0054 protein. For instance, the concentration of magnesium(II) could be greater than about 5 mM, greater than about 10 mM, greater than 20 mM, etc. In one embodiment, the solution containing the UPF0054 protein and magnesium(II) at a concentration sufficient to inhibit the UPF0054 protein could be diluted to reduce the concentration of magnesium(II) in order to activate the enzyme. For example, the UPF0054 protein may be stored in a concentrated stock solution that is diluted upon addition to a reaction mixture.

In some aspects, microbial UPF0054 proteins may be analyzed as pharmaceutical targets. As discussed above, it is believed that the UPF0054 proteins are highly conserved, and may be important in proper ribosomal activity, e.g., due to the effects on 16S rRNA maturation. Accordingly, the present invention discloses, in one set of embodiments, that reducing the activity of microbial UPF0054 proteins may lead to lethality or increased sensitivity of affected microbes to drugs or other therapeutic agents. Thus, without wishing to be bound by any theory, it is believed that inhibiting these proteins may be beneficial for the treatment of microbial infections, as discussed herein. In some cases, due to their highly conserved nature, the treatment may be one that is applicable to a range of microbial infections, e.g., as a broad-spectrum antibiotic. In some embodiments, combining the inhibition of UPF0054 with administering an antimicrobial, such as an antibiotic, can lead to an effective or more effective treatment of microbial infections than the antimicrobial alone.

Inhibition of the activity of the UPF0054 protein, and/or of a cell containing the UPF0054 protein, may be partial or total. In some cases, inhibition (whether partial or total) of a cell expressing the UPF0054 protein may result in a decrease in cell activity or viability of the cell. For example, an inhibitor of UPF0054 protein may be administered to a plurality of cells, and at least about 10%, at least about 20%, at least about 30%, at least about 40%, at least about 50%, at least about 60%, at least about 70%, at least about 80%, at least about 90%, or at least about 100% of the cells may die as a result of exposure to the inhibitor. In another embodiment, the inhibition may cause the cells to decrease in growth, e.g., in growth rate, relative to cells not exposed to the inhibitor of the UPF0054 protein. For example, the growth rate may decrease by at least about 10%, at least about 20%, at least about 30%, at least about 40%, at least about 50%, at least about 60%, at least about 70%, at least about 80%, at least about 90%, or at least about 100%, relative to unexposed cells. In another set of embodiments, the inhibitor inhibits UPF0054 protein activity, for example, by inhibiting a UPF0054 protein-dependent cellular pathway, and/or a cellular target of the UPF0054 protein. The inhibitor, in yet another set of embodiments, is able to inhibitor inhibit translation and/or transcription of the UPF0054 protein, or is able to inhibit a conformational state of the UPF0054 protein. For instance, in the presence of the inhibitor, expression by a cell of UPF0054 protein activity may decrease by at least about 10%, at least about 20%, at least about 30%, at least about 40%, at least about 50%, at least about 60%, at least about 70%, at least about 80%, at least about 90%, or at least about 100%, relative to unexposed cells. As another example, the inhibitor may be at least partially prevent the UPF0054 protein from properly folding into its native configuration.

In one embodiment, antisense oligonucleotides can be used to inhibit UPF0054 protein expression by binding to mRNA transcripts and preventing their translation. The antisense oligonucleotide may be antisense to all, or a portion of, the UPF0054. For instance, the antisense oligonucleotide may be antisense to any one of the genes coding for SEQ ID NOs: 1-95. Those of ordinary skill in the art will be able to readily generate such sequences (e.g., as being substantially complementary to any of SEQ ID NOs: 1-95) using no more than routine skill. In some instances, the antisense oligonucleotide may be a DNA, which can bind to an mRNA transcript to form a DNA/RNA hydrid duplex that is degradable by RNase H.

In some aspects of the invention, assays are provided for determining properties of a UPF0054 protein and identifying compositions that have an effect on a property of a UPF0054 protein. For example, the composition may be one that inhibits activity of the UPF0054 protein, and the composition may be one that can be used as an antibiotic, or used in conjunction with another antibiotic. In some cases, the assays may be conducted as high-throughput assays, e.g., involving the screening of a plurality of compositions simultaneously. For instance, the assays may be conducted using 96 well plates, 384 well plates, or using other commonly-available high-throughput techniques. In other cases, however, the assays are not necessarily conducted as high-throughput assays.

In some embodiments, an in vitro or an in vivo assay can be used to screen compounds for the ability to inhibit a UPF0054 protein, e.g., for use as an inhibitor. For example, fluorescence polarization of an RNA oligomer connected to a fluorophore can be assayed to determine the activity of a UPF0054 protein, since cleavage of the RNA oligomer by the UPF0054 protein can result in a shorter RNA oligomer, which leads to a change in fluorescence polarization. Contacting the UPF0054 protein with a candidate compound and performing the above described assay may allow the effect of the compound on the activity of the UPF0054 protein to be determined, for example by observing a change in the fluorescence polarization over time in the presence versus absence of the candidate compound. Variations to this assay would be apparent to those skilled in the art. For instance, the RNA substrate may be a radiolabeled RNA oligomer. The assay could be used to examine the change in length of the RNA oligomer over time by, for example, separating the RNA substrate molecules by gel electrophoresis and then determining the amounts or concentrations of radioactivity at locations throughout the gel. In other embodiments, assays for determining the effect of a target compound on one UPF0054 protein versus another UPF0054 protein in cells are provided. For example, two strains of E. coli could be provided, each strain expressing a different indicator such as a different fluorescent proteins. A fluorimeter or other suitable device can be used to determine the amount of fluorescence emitted by the fluorescent protein. In some embodiments, the amount of fluorescence emitted from a cell culture is proportional to the number of viable cells. By monitoring the change in the amount of fluorescence emitted by a cell culture as a function of time, the growth rate of the cells can be determined. Other methods for measuring the growth rate of cells, for example, by determining the optical density of a liquid cell culture using 600 nm light and/or by plating an aliquot of cells from various time points on an agar/nutrient plate and determining the number of colony forming units (cfu), are known to those skilled in the art. In some cases, e.g., as discussed below, an assay may be conducted in the presence of other agents, for example, antibiotics (e.g., ampicillin, cefotaxime, kasugamycin, erythromycin, etc.), and/or compositions suspected to alter the effects or properties of UPF0054 proteins, and/or other cells (e.g., other cell types, cells expressing other UPF0054 proteins, etc)., or the like.

In some embodiments, two or more strains may be studied to determine UPF0054 protein expression or activity. The two or more strains may be from the same species, or from different species. In one embodiment, for example, a first strain may express a first UPF0054 protein and a first fluorescent protein having a particular fluorescence emission peak, and a second strain may express a second UPF0054 protein, the second UPF0054 protein having a different amino acid sequence from the first UPF0054 protein, and a second fluorescent protein having a fluorescence emission peak different from that of the first fluorescent protein. Both strains may have the native UPF0054 protein inactivated. As another example, one strain expresses the native UPF0054 protein and another strain has the native UPF0054 protein inactivated. In some embodiments, the inactivated UPF0054 protein may have some or all of the DNA sequence coding for the protein removed, essentially eliminating the activity of the protein. In another embodiment, the inactivated UPF0054 protein may be a mutated UPF0054 protein with essentially no activity. As yet another example, a first strain may express a first UPF0054 protein extrachromosomally, and a second strain may express a second UPF0054 protein extrachromosomally, or the second UPF0054 protein having an amino acid sequence different from the first UPF0054 protein, etc. In another set of embodiments, the DNA encoding at least one UPF0054 protein may be integrated into the host cell genome. In another embodiment, the DNA encoding at least one UPF0054 protein may be integrated into a plasmid.

In some cases, cells or strains such as those described above can be grown, in certain embodiments, in the presence of a toxin, such as an antibiotic, e.g. cefotaxime, kasugamycin, erythromycin, ampicillin, etc., or others as described herein. For example, two (or more) cell types or strains may be grown at a level so that the growth rates of the two cells are similar and so that inhibition of an expressed UPF0054 protein would be toxic to the cell expressing the UPF0054 protein. The cell types may be, for example, from different species, or from the same species but one or both may be genetically engineered, e.g., to express a non-endogenous UPF0054 protein, to change the expression level of an endogenous UPF0054 protein, etc. In some cases, a strain may be a wild type strain, a strain that has been engineered to express an endogenous UPF0054 protein at a substantially different level than the wild type, a strain that has been engineered to express a non-endogenous UPF protein, or the like. In some cases, a strain engineered to express a non-endogenous UPF protein may also be engineered to not express the endogenous UPF0054 protein, and/or to express the endogenous UPF0054 protein at a reduced level.

The growth rates of the cells can be determined using, for example, the methods described above. According to the above embodiments, if the strains are treated with an agent that inhibits the UPF0054 protein in one of the strains, a difference in growth rate between the strains can result, which may lead to a change in the ratio of the fluorescence from a first strain to the fluorescence from a second strain, thereby enabling inhibition of at least one of the UPF0054 proteins to be determined. In some embodiments, a strain may contain a mammalian UPF0054 homolog, e.g. the human UPF0054 homolog, C21orf57, or a strain may contain a UPF0054 protein from a pathogenic organism, e.g., YbeY.

The assay may also be used, in some embodiments, with a specific organism as a screen to determine agents that cross the cell membrane of that organism in addition to showing activity against a UPF0054 protein. More than two strains may be used in the assay, in some cases each having a different indicator, to evaluate multiple UPF0054 proteins. As another example, a screening method may be conducted to identify compositions that have a greater effect on a first UPF0054 protein relative to a second UPF0054 protein, for example, a greater effect on YbeY relative to the human homolog C21orf57. Other forms of indicators may be used as an output for determining the relative growth rate of cells. For instance, cells could be plated before and after treatment with an agent, with cells from the first strain expressing functional beta-galactosidase and cells from the second strain expressing a non-functional beta-galactoside, e.g. a mutant, or not expressing beta-galactosidase. Cells from the first strain would have a color when plated in the presence of an appropriate indicator, and cells from the second strain would have a color that is distinguishable from the cells of the first strain.

A non-limiting example of a method of identifying inhibitors in vivo follows. In some cases, such methods may be used as part of a high-throughput screen. For example, one or more wells of a microwell plate may have a series of potential compositions added to them suspected of interacting with UPF0054 protein activity (e.g., of YbeY activity, of a YbeY-dependent pathway in a cell, of human C21orf57 activity, etc.). The effect of the compositions on the UPF0054 activity to an RNA oligomer may be assessed, e.g., using fluorescence, ELIZA, FRET, or other known techniques, to determine whether the compositions are effective, e.g., at at least partially inhibiting UPF0054 activity. In some cases, the selective activity of the composition may be assessed, e.g., by determining the level of activity of a first UPF0054 protein relative to a second UPF0054 protein. For example, as discussed herein, compositions having relatively selective activity to YbeY relative to human C21orf57 may be selected in an assay. As a specific example, inhibition (whether total or partial) of YbeY may sensitize certain cells, such as E. coli, to stresses including various antibiotics such as ampicillin, cefotaxime, kasugamycin, erythromycin, or the like, or others as described herein.

As a specific example, a wild type E. coli strain and an E. coli YbeY mutant complemented with human C21orf57 in LB or minimal medium containing 100 micrograms/ml of ampicillin to maintain plasmid and 200 micrograms/ml of kasugamyin as a stress agent may be studied, or exposed to compositions suspected of being able to interact with this system, etc. Kasugamycin may be useful in some cases since it may not substantially differentially inhibit the growth of either wild type E. coli strain or E. coli YbeY mutant complemented with human C21orf57 strain, but does inhibit the growth of a YbeY mutant.

As another example, two (or more) samples with small molecules (e.g., having a molecular weight of less than about 1000 Da) may be studied, where one corresponds to a WT strain (E. coli with native YbeY and kasugamycin), and the other correspopnds to C21orf57 complemented AYbeY strain (vector, C21orf57, and kasugamycin). In some cases, the samples may contain a wild-type (WT) strain (for example, bacteria with functional bacterial YbeY), a strain lacking YbeY and complemented with human C21orf57, or the like.

In another example, differential reduction of growth between a WT strain (e.g., E. coli with native YbeY+kasugamycin) and a C21orf57 complemented ΔYbeY strain (e.g,. vector, C21orf57, and kasugamycin) in the presence of candidate compositions, compared to those without the composition, is determined. This differential reduction, for example, may be compared with a positive control, for example, a ΔYbeY mutant in the presence of kasugamycin.

As a specific non-limiting example, in one in vivo screen, 96 or 384 microwell plates may be used, some of which wells may contain 50-100 microliters of solution. E. coli or other species may be used in some cases, as previously discussed. 100 n1 of 5 mg/ml solution of candidate compositions may be introduced or pinned into the wells, and the cells in some cases allowed to grow at 370 C for 24 hours or longer. The condition of the cells may be determined, e.g., using growth readings (for example, OD600) at various time points (e.g., 0, 12 and 24 hours) after addition of the candidate compositions. In some cases, suitable positive and/or negative controls may be used, for example, ΔYbeY in the presence of kasugamycin, and/or the E. coli genotypes with kasugamycin, as example positive and negative controls respectively.

In one set of embodiments, FRET techniques or other fluorescence techniques known to those of ordinary skill in the art may be used in various assays of the invention. As a non-limiting example, in one embodiment, an oligomer, such as an RNA oligomer, may be coupled to a fluorophore-quencher pair. For instance, the fluorophore may be fluorescein and the quencher may be DY547. The fluorophore may be attached to the 3′end of the oligomer and the quencher attached to 5′ end. In some cases, the UPF0054 protein may be active and cut the oligomer, resulting in a change in fluorescence, compared to intact oligomer. For instance, upon removal of the quencher, the fluorescence may increase. Accordingly, in one set of embodiments, a change in fluorescence may be used to assay activity of the UPF0054 protein on the oligomer.

As previously discussed, in one set of embodiments, a cell, such as a bacteria, is exposed to an inhibitor of a member of UPF0054 protein superfamily, and an antibiotic. Without wishing to be bound by any theory, it is believed that exposure of the cell to the inhibitor may inhibit, partially or totally, function of the UPF0054 protein within the cell. In some cases, the inhibition may result in the death of the cell; in other cases, the inhibition may weaken the cell in some fashion, for example, causing the cell to become more susceptible to an antibiotic or other agent. Non-limiting examples of antibiotics are discussed below. In some cases, the inhibitor and the antibiotic may be administered to a subject, such as a human subject. For example, the subject may be infected with bacteria, and the inhibitor and the antibiotic may be administered to the subject to treat the infection. Treatment of the subject may result, for example, in a decrease in the number of bacteria within the subject, and/or a decrease in the severity of the symptoms exhibited by the subject due to the bacteria.

Examples of antibiotics include, but are not limited to, tetracycline antibiotics, such as chlortetracycline, oxytetracycline, tetracycline, demethylchlortetracycline, metacycline, doxycycline, minocycline and rolitetracycline; aminoglysodes, such as kanamycin, amikacin, gentamicin C₁ₐ, C₂, C_{2b} or C₁, sisomicin, netilmicin, spectinomycin, streptomycin, tobramycin, neomycin B, dibekacin and kanendomycin; macrolides, such as maridomycin and erythromycin; lincomycins, such as clindamycine and lincomycin; penicillanic acid (6-APA)- and cephalosporanic acid (7-ACA)-derivatives having (6β- or 7β-acylamino groups, respectively, which are present in fermentatively, semi-synthetically or totally synthetically obtainable 6β-acylaminopenicillanic acid or 7β-acylaminocephalosporanic acid derivatives and/or 7β-acylaminocephalosporanic acid derivatives that are modified in the 3-position, such as penicillanic acid derivatives that have become known under the names penicillin G or V, such as phenethicillin, propicillin, nafcillin, oxycillin, cloxacillin, dicloxacillin, flucloxacillin, cyclacillin, epicillin, mecillinam, methicillin, azlocillin, sulbenicillin, ticarcillin, mezlocillin, piperacillin, carindacillin, azidocillin or ciclacillin, and cephalosporin derivatives that have become known under the names cefaclor, cefuroxime, cefazlur, cephacetrile, cefazolin, cephalexin, cefadroxil,cephaloglycin, cefoxitin, cephaloridine, cefsulodin, cefotiam, ceftazidine, cefonicid, cefotaxime, cefmenoxime, ceftizoxime, cephalothin, cephradine, cefamandol, cephanone, cephapirin, cefroxadin, cefatrizine, cefazedone, ceftrixon and ceforanid; and other β-lactam antibiotics of the clavam, penem and carbapenen type, such as moxalactam, clavulanic acid, nocardicine A, sulbactam, aztreonam and thienamycin; and antibiotics of the bicozamycin, novobiocin, chloramphenicol or thiamphenicol, rifampicin, fosfomycin, colistin and vancomycin. Other antibiotics potentially useful in the invention include broad spectrum antibiotics and narrow spectrum antibiotics. Antibiotics that are effective against a single organism or disease and not against other types of bacteria, are generally referred to as limited spectrum antibiotics. In general, antibacterial agents are cell wall synthesis inhibitors, such as beta-lactam antibiotics (e.g., carbapenems and cephalolsporins, including cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, moxalactam, etc.), natural penicillins, semi-synthetic penicillins (e.g., ampicillin, carbenicillin, oxacillin, azlocillin, mezlocillin, piperacillin, methicillin, dicloxacillin, nafcillin, etc.), ampicillin, clavulanic acid, cephalolsporins, bacitracin, etc.; cell membrane inhibitors (e.g., polymyxin, amphotericin B, nystatin, imidazoles including clotrimazole, miconazole, ketoconazole, itraconazole, fluconazole, etc.); protein synthesis inhibitors (e.g., tetracyclines, chloramphenicol, macrolides such as erythromycin, aminoglycosides such as streptomycin, rifampins, ethambutol, streptomycin, kanamycin, tobramycin, amikacin, gentamicin, tetracyclines (e.g., tetracycline, minocycline, doxycycline, and chlortetracycline, etc.), erythromycin, roxithromycin, clarithromycin, oleandomycin, azithromycin, chloramphenicol, etc.); nucleic acid synthesis or functional inhibitors (e.g., quinolones, co-trimoxazole, rifamycins, etc.); and/or competitive inhibitors (e.g., sulfonamides such as gantrisin, trimethoprim, etc.).

Another aspect of the present invention is generally directed to inhibitors of a member of UPF0054 protein superfamily. In some cases, inhibitors such as these are found using techniques for screening, etc., such as those described herein. In certain embodiments, these inhibitors are small molecules.

In some embodiments, the UPF0054 protein superfamily inhibitor is oxytetracycline, demeclocycline, or an antibiotic substance or substances belonging to the “tetracycline” or “tetracycline derivative” class of compounds. Tetracycline compounds include tetracycline and other tetracycline family members such as, chlortetracycline, oxytetracycline, demeclocycline, methacycline, doxycycline, minocycline, and sancycline. Additional tetracycline compounds of the invention can be found, for example, in U.S. patent application Ser. No. 09/234,847, and U.S. Pat. Nos. 5,834,450; 5,532,227; 5,789,395; 5,639,742 and German patents DE 28 14 974 and DE 28 20 983. The entire contents of the aforementioned applications and patents are hereby expressly incorporated herein by reference.

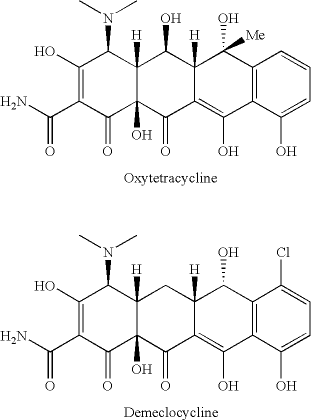

In further embodiments, the UPF0054 protein superfamily inhibitor is cefotaxime or an antibiotic substance or substances belonging to the cephalosporin class of compounds. The known class of cephalosporanic antibiotics are disclosed, for example, in U.S. Pat. No. 4,098,888 (1978) as well as numerous other patents and other publications. This class of antibiotics is characterized by the presence of an oximino group and a 2-aminothiazolyl heterocyclic ring in the 7-acylamido side-chain attached to the cephalosporin nucleus. This class of compounds is also characterized by suitable substituents at the 3-position of the cephalosporin nucleus. The entire contents of the aforementioned patent are hereby expressly incorporated herein by reference.

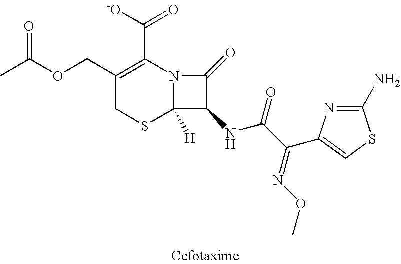

In some embodiments, the UPF0054 protein superfamily inhibitor is nortriptyline or an antideptressant substance or substances belonging to the tricyclic antidepressant (TCA) class of compounds. TCAs are a recognized class of structurally related drugs used for the treatment of depression. (The Pharmacological Basis of Therapeutics, 5th ed., ed. Goodman and Gilman, MacMillan Publ. Co. (New York 1975) pp. 174 et seq.) All of the drugs have an annealated three ring nucleus, a “tricyclic” nucleus, which is often, but by no means exclusively, dibenzazepinyl, dibenzocycloheptadienyl, dibenzoxepinyl, or phenothiazinyl in nature. A further common feature found in this class of compounds is the presence of a side chain of substantial length off one of the atoms, usually carbon or nitrogen, in the central ring of the tricylic nucleus. The present invention relates to members of this group such as imipramine, desipramine, amitriptyline, nortriptyline, protriptylene, trimipramine, chlomipramine, doxepin, amoxapine, clomipramine, maprotriline, and carbamazepine, as well as biologically active or therapeutically active derivatives and metabolites thereof, and their pharmaceutically effective salts and esters, such as, but not limited to their hydrochlorides, maleates, tartrates and lactates.

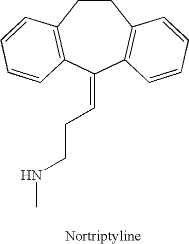

In certain embodiments, the UPF0054 protein superfamily inhibitor is of the formula:

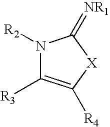

wherein X is O or S; each of R₁, R₂, R₃, and R₄ is independently hydrogen or cyclic or acyclic, branched or unbranched, substituted or unsubstituted aliphatic; and pharmaceutically acceptable salts thereof.

In certain embodiments, X is O. In other embodiments, X is S. In certain embodiments, R₁ is hydrogen. In certain embodiments, R₁ is C₁-C₆ alkyl. In certain embodiments, R₂ is hydrogen. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is butyl. In certain embodiments, R₃ is hydrogen. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is butyl. In certain embodiments, R₄ is hydrogen. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is butyl. In certain embodiments, R₄ is tert-butyl.

In certain embodiments, the compound is of the formula:

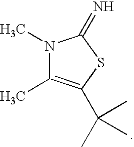

In certain embodiments, the UPF0054 protein superfamily inhibitor is of the formula:

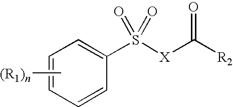

wherein X is NH, NR, O, S, CHR, CR₂, or CH₂; n is an integer between 0 and 5, inclusive; each occurrence of R is independently hydrogen, a halogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N₃; —N(R_{B})₂; —NHC(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrene of R_{G} is independently a hydrogen, a protecting group, an aliphatic moiety, a heteroaliphatic moiety, an acyl moiety; an aryl moiety; a heteroaryl moiety; alkoxy;

aryloxy; alkylthio; arylthio; amino, alkylamino, dialkylamino, heteroaryloxy; or heteroarylthio moiety; and pharmaceutically acceptable salts thereof.

In certain embodiments, X is NH. In certain embodiments, X is NR. In certain embodiments, X is O. In certain embodiments, X is S. In certain embodiments, X is CH₂. In certain embodiments, n is 1. In certain embodiments, n is 2. In certain embodiments, n is 3. In certain embodiments, R₁ is hydrogen. In certain embodiments, R₁ is halogen. In certain embodiments, R₁ is alkyl. In certain embodiments, R₁ is C₁-C₆ alkyl. In certain embodiments, R₁ is methyl. In certain embodiments, R₁ is ethyl. In certain embodiments, R₁ is propyl. In certain embodiments, R₁ is butyl. In certain embodiments, R₁ is halogen. In certain embodiments, R₁ is hydroxyl. In certain embodiments, R₁ is alkoxy. In certain embodiments, R₁ is thioxy. In certain embodiments, R₁ is alkylthioxy. In certain embodiments, R₁ is amino. In certain embodiments, R₁ is alkylamino. In certain embodiments, R₁ is dialkylamino. In certain embodiments, R₁ is acyl.

In certain embodiments, R₂ is hydrogen. In certain embodiments, R₂ is alkyl. In certain embodiments, R₂ is substituted alkyl. In certain embodiments, R₂ is unsubstituted alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is butyl. In certain embodiments, R₂ is hydroxyl. In certain embodiments, R₂ is alkoxy. In certain embodiments, R₂ is thioxy. In certain embodiments, R₂ is alkylthioxy. In certain embodiments, R₂ is amino. In certain embodiments, R₂ is alkylamino. In certain embodiments, R₂ is dialkylamino. In certain embodiments, R₂ is acyl. In certain embodiments, R₂ is hydroxymethyl. In certain embodiments, R₂ is hydroxyethyl. In certain embodiments, R₂ is aminomethyl. In certain embodiments, R₂ is aminoethyl. In certain embodiments, R₂ is —CH₂CN. In certain embodiments, R₂ is —CH₂CH₂CN. In certain embodiments, R₂ is —CH₂SH. In certain embodiments, R₂ is —CH₂CH₂SH.

In certain embodiments, the UPF0054 protein superfamily inhibitor is of the formula:

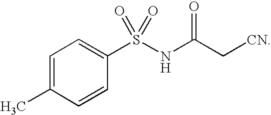

In certain other embodiments, the UPF0054 protein superfamily inhibitor is of the formula:

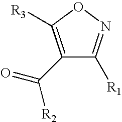

wherein R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or to unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N₃; —N(R_{B})₂; —NHC(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N₃; —N(R_{C})₂; —NHC(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, R₁ is substituted or unsubstituted heteroaryl. In certain embodiments, R₁ is substituted heteroaryl. In certain embodiments, R₁ is unsubstituted heteroaryl. In certain embodiments, R₁ is a substituted or unsubstituted 5-membered heteroaryl. In certain embodiments, R₁ is a substituted or unsubstituted thiazolyl, oxazolyl, imidazolyl, thiophenyl, furanyl, triazolyl, pyrrolyl, tetrahydropyrrolyl, tetrahydrofuranyl, isoxazolyl, isothiazoleyl thiopheneyl, pyrazolyl, dithiolanyl, dioxolanyl, thiazolidineyl, and the like. In certain embodiments, R₁ is a substituted or unsubstituted 6-membered heteroaryl. In certain embodiments, R₁ is a substituted or unsubstituted pyridinyl. In certain embodiments, R₁ is a substituted or unsubstituted bicyclic heteroaryl. In certain embodiments, R₁ is a substituted or unsubstituted tricyclic heteroaryl. In certain embodiments, R₁ is substituted or unsubstituted aryl. In certain embodiments, R₁ is substituted aryl. In certain embodiments, R₁ is unsubstituted aryl. In certain embodiments, R₁ is unsubstituted phenyl. In certain embodiments, R₁ is substituted phenyl. In certain embodiments, R₁ is monosubstituted phenyl. In certain embodiments, R₁ is disubstituted phenyl. In certain embodiments, R₁ is trisubstituted phenyl. In certain embodiments, R₁ is 2-halophenyl. In certain embodiments, R₁ is 2-chlorophenyl.

In certain embodiments, R₂ is —OR_{B}. In certain embodiments, R₂ is —OCH₃. In certain embodiments, R₂ is —OH. In certain embodiments, R₂ is —NH₂. In certain embodiments, R₂ is —NHR_{B}. In certain embodiments, R₂ is —N(R_{B})₂. In certain embodiments, each occurrence of R_{B} is hydrogen or C₁-C₆ alkyl. In certain embodiments, each occurrence of R_{B} is hydrogen or C₁-C₆ alkenyl. In certain embodiments, each occurrence of R_{B} is hydrogen, C₁-C₆ alkyl, or C₁-C₆ alkenyl. In certain embodiments, R₂ is —N(CH₃)₂. In certain embodiments, R₂ is —N(Et)₂. In certain embodiments, R₂ is —N(Pr)₂. In certain embodiments, R₂ is alkyl. In certain embodiments, R₂ is substituted alkyl. In certain embodiments, R₂ is unsubstituted alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is butyl.

In certain embodiments, R₃ is hydrogen. In certain embodiments, R₃ is halogen. In certain embodiments, R₃ is alkyl. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is butyl.

In certain embodiments, the UPF0054 protein superfamily inhibitor is of the formula:

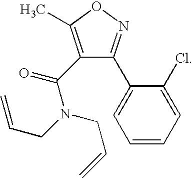

In certain embodiments, the UPF0054 protein superfamily inhibitor is of formula:

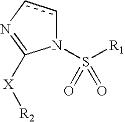

wherein the dashed line represents the presence or the absence of a bond; X is O, S, NH, NR, CH₂, or C(R)₂; each occurrence of R is independently hydrogen, a halogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N₃; —N(R_{A})₂; —NHC(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N₃; —N(R_{B})₂; —NHC(═O)R_{B}; —NR_{B}C(═O)N(R_{B}₂; —OC(═)OR_{B}; —OC(═O)R_{B}; —OC(═O)N(R_{B})₂; —NR_{B}C(═O)OR_{B}; or —C(R_{B})₃; wherein each occurence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, the compound is of the formula:

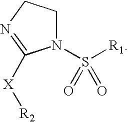

In certain embodiments, X is O. In certain embodiments, X is S. In certain embodiments, X is NH. In certain embodiments, X is —CH₂—. In certain embodiments, R₁ is hydrogen. In certain embodiments, R₁ is halogen. In certain embodiments, R₁ is alkyl. In certain embodiments, R₁ is C₁-C₆ alkyl. In certain embodiments, R₁ is methyl. In certain embodiments, R₁ is ethyl. In certain embodiments, R₁ is propyl. In certain embodiments, R₁ is butyl. In certain embodiments, R₁ is substituted or unsubstituted heteroaryl. In certain embodiments, R1 is substituted heteroaryl. In certain embodiments, R₁ is unsubstituted heteroaryl. In certain embodiments, R₁ is a substituted or unsubstituted 5-membered heteroaryl. In certain embodiments, R₁ is a substituted or unsubstituted thiazolyl, oxazolyl, imidazolyl, thiophenyl, furanyl, triazolyl, pyrrolyl, tetrahydropyrrolyl, tetrahydrofuranyl, isoxazolyl, isothiazoleyl thiopheneyl, pyrazolyl, dithiolanyl, dioxolanyl, thiazolidineyl, and the like. In certain embodiments, R₁ is a substituted or unsubstituted 6-membered heteroaryl. In certain embodiments, R₁ is a substituted or unsubstituted pyridinyl. In certain embodiments, R₁ is a substituted or unsubstituted bicyclic heteroaryl. In certain embodiments, R₁ is a substituted or unsubstituted tricyclic heteroaryl. In certain embodiments, R₁ is substituted or unsubstituted aryl. In certain embodiments, R₁ is substituted aryl. In certain embodiments, R₁ is unsubstituted aryl. In certain embodiments, R₁ is unsubstituted phenyl. In certain embodiments, R₁ is substituted phenyl. In certain embodiments, R₁ is monosubstituted phenyl. In certain embodiments, R₁ is disubstituted phenyl. In certain embodiments, R₁ is trisubstituted phenyl. In certain embodiments, R₁ is 4-halo phenyl. In certain embodiments, R₁ is 4-fluoro phenyl.

In certain embodiments, R₂ is hydrogen. In certain embodiments, R₂ is halogen. In certain embodiments, R₂ is alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is butyl. In certain embodiments, R₂ is alkenyl. In certain embodiments, R₂ is alkynyl. In certain embodiments, R₂ is arylalkyl. In certain embodiments, R₂ is heteroarylalkyl. In certain embodiments, R₂ is substituted or unsubstituted heteroaryl. In certain embodiments, R₂ is substituted heteroaryl. In certain embodiments, R₂ is unsubstituted heteroaryl. In certain embodiments, R₂ is a substituted or unsubstituted 5-membered heteroaryl. In certain embodiments, R₂ is a substituted or unsubstituted thiazolyl, oxazolyl, imidazolyl, thiophenyl, furanyl, triazolyl, pyrrolyl, tetrahydropyrrolyl, tetrahydrofuranyl, isoxazolyl, isothiazoleyl thiopheneyl, pyrazolyl, dithiolanyl, dioxolanyl, thiazolidineyl, and the like. In certain embodiments, R₂ is a substituted or unsubstituted 6-membered heteroaryl. In certain embodiments, R₂ is a substituted or unsubstituted pyridinyl. In certain embodiments, R₂ is a substituted or unsubstituted bicyclic heteroaryl. In certain embodiments, R₂ is a substituted or unsubstituted tricyclic heteroaryl. In certain embodiments, R₂ is substituted or unsubstituted aryl. In certain embodiments, R₂ is substituted aryl. In certain embodiments, R₂ is unsubstituted aryl. In certain embodiments, R₂ is unsubstituted phenyl. In certain embodiments, R₂ is substituted phenyl. In certain embodiments, R₂ is monosubstituted phenyl. In certain embodiments, R₂ is disubstituted phenyl. In certain embodiments, R₂ is trisubstituted phenyl.

In certain embodiments, R₂ is of the formula:

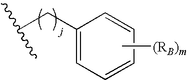

wherein j is an integer between 0 and 20, inclusive; m is an integer between 0 and 5, inclusive; and each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N₃; —N(R′)₂; —NHC(═O)R′; —NR′C(═O)N(R′)₂; —OC(═O)OR′; —OC(═O)R′; —OC(═O)N(R′)₂; —NR′C(═O)OR′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, j is an integer between 1 and 12, inclusive. In certain embodiments, j is an integer between 1 and 6, inclusive. In certain embodiments, j is 0. In certain embodiments, j is 1. In certain embodiments, j is 2. In certain embodiments, j is 3. In certain embodiments, m is 1. In certain embodiments, m is 2. In certain embodiments, m is 3. In certain embodiments, R_{B} is hydrogen. In certain embodiments, R_{B} is halogen. In certain embodiments, R_{B} is fluoro. In certain embodiments, R_{B} is chloro. In certain embodiments, R_{B} is alkyl. In certain embodiments, R_{B} is C₁-C₆ alkyl. In certain embodiments, R_{B} is methyl. In certain embodiments, R_{B} is ethyl. In certain embodiments, R_{B} is propyl. In certain embodiments, R_{B} is butyl. In certain embodiments, R_{B} is halogen. In certain embodiments, R_{B} is hydroxyl. In certain embodiments, R_{B} is alkoxy. In certain embodiments, R_{B} is thioxy. In certain embodiments, R_{B} is alkylthioxy. In certain embodiments, R_{B} is amino. In certain embodiments, R_{B} is alkylamino. In certain embodiments, R_{B} is dialkylamino. In certain embodiments, R_{B} is acyl.

In certain embodiments, R₂ is of the formula:

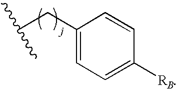

In certain embodiments, the UPF0054 protein superfamily inhibitor is of the formula:

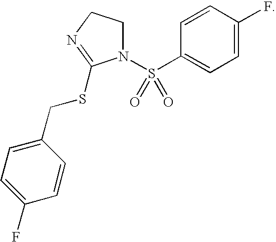

In some embodiments of the invention, the composition may include homologs, analogs, derivatives, enantiomers and/or functionally equivalent compositions thereof of the inhibitors discussed herein. Such homologs, analogs, derivatives, enantiomers and functionally equivalent compositions thereof of the compositions may also be used in any of the assays described above. It will be understood that the skilled artisan will be able to manipulate the conditions in a manner to prepare such homologs, analogs, derivatives, enantiomers and functionally equivalent compositions. Homologs, analogs, derivatives, enantiomers and/or functionally equivalent compositions which are about as effective or more effective than the parent compound are also intended for use in the methods of the invention. Synthesis of such compositions may be accomplished through typical chemical modification methods such as those routinely practiced in the art.

In one aspect of the invention, an inhibitor such as those described above may be administered to a subject. In some cases, the inhibitor may act as an antibiotic, and/or the inhibitor may be used with an antibiotic such as the ones described herein.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

The compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

Where an isomer/enantiomer is preferred, it may, in some embodiments, be provided substantially free of the corresponding enantiomer, and may also be referred to as “optically enriched.” “Optically enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); Wilen, Tables of Resolving Agents and Optical Resolutions p. 268 (E. L. Eliel, Ed., Univ. of Notre Dame Press, Notre Dame, Ind. 1972).

It will be appreciated that the compounds of the present invention, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include substitution with all permissible substituents of organic compounds, any of the substituents described herein (for example, aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, etc.), and any combination thereof (for example, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like) that results in the formation of a stable moiety. The present invention contemplates any and all such combinations in order to arrive at a stable substituent/moiety. Additional examples of generally applicable substitutents are illustrated by the specific embodiments shown in the Examples, which are described herein. For purposes of this invention, heteroatoms such as nitrogen may have hydrogen substituents and/or any suitable substituent as described herein which satisfy the valencies of the heteroatoms and results in the formation of a stable moiety.

The term “acyl,” as used herein, refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “aliphatic,” as used herein, includes both saturated and unsaturated, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms. Aliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms. In another embodiment, the alkyl group employed contains 1-15 carbon atoms. In another embodiment, the alkyl group employed contains 1-10 carbon atoms. In another embodiment, the alkyl group employed contains 1-8 carbon atoms. In another embodiment, the alkyl group employed contains 1-5 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more sustitutents. Alkyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkenyl,” as used herein, denotes a monovalent group derived from a straight- or branched-chain hydrocarbon moiety having at least one carbon-carbon double bond by the removal of a single hydrogen atom. In certain embodiments, the alkenyl group employed in the invention contains 2-20 carbon atoms. In some embodiments, the alkenyl group employed in the invention contains 2-15 carbon atoms.

In another embodiment, the alkenyl group employed contains 2-10 carbon atoms. In still other embodiments, the alkenyl group contains 2-8 carbon atoms. In yet other embodiments, the alkenyl group contains 2-5 carbons. Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms. In some embodiments, the alkynyl group employed in the invention contains 2-15 carbon atoms. In another embodiment, the alkynyl group employed contains 2-10 carbon atoms. In still other embodiments, the alkynyl group contains 2-8 carbon atoms. In still other embodiments, the alkynyl group contains 2-5 carbon atoms. Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “amino,” as used herein, refers to a group of the formula (—NH₂). A “substituted amino” refers either to a mono-substituted amine (—NHR^{h}) of a disubstitued amine (—NR^{h}₂), wherein the R^{h} substituent is any substitutent as described herein that results in the formation of a stable moiety (e.g., a suitable amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). In certain embodiments, the R^{h} substituents of the di-substituted amino group (—NR^{h}₂) form a 5- to 6-membered hetereocyclic ring.

The term “alkoxy” refers to a “substituted hydroxyl” of the formula (—OR′), wherein Rⁱ is an optionally substituted alkyl group, as defined herein, and the oxygen moiety is directly attached to the parent molecule.

The term “alkylthioxy” refers to a “substituted thiol” of the formula (—SR^{r}), wherein R^{r} is an optionally substituted alkyl group, as defined herein, and the sulfur moiety is directly attached to the parent molecule.

The term “alkylamino” refers to a “substituted amino” of the formula (—NR^{h}₂), wherein R^{h} is, independently, a hydrogen or an optionally subsituted alkyl group, as defined herein, and the nitrogen moiety is directly attached to the parent molecule.

The term “aryl,” as used herein, refer to stable aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but not limited to, phenyl, biphenyl, naphthyl, and the like, which may bear one or more substituents. Aryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “aryloxy” refers to a “substituted hydroxyl” of the formula (—ORⁱ), wherein Rⁱ is an optionally substituted aryl group, as defined herein, and the oxygen moiety is directly attached to the parent molecule.

The term “arylamino,” refers to a “substituted amino” of the formula (—NR^{h}₂), wherein R^{h} is, independently, a hydrogen or an optionally substituted aryl group, as defined herein, and the nitrogen moiety is directly attached to the parent molecule.

The term “cyano,” as used herein, refers to a group of the formula (—CN).

The terms “halo” and “halogen” as used herein refer to an atom selected from fluorine (fluoro, —F), chlorine (chloro, —Cl), bromine (bromo, —Br), and iodine (iodo, —I).

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that contain one or more oxygen, sulfur, nitrogen, phosphorus, or silicon atoms, e.g., in place of carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl”, “heteroalkynyl”, and the like. Furthermore, as used herein, the terms “heteroalkyl”, “heteroalkenyl”, “heteroalkynyl”, and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms. Heteroaliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroalkyl,” as used herein, refers to an alkyl moiety, as defined herein, which contain one or more oxygen, sulfur, nitrogen, phosphorus, or silicon atoms, e.g., in place of carbon atoms.

The term “heterocyclic,” “heterocycles,” or “heterocyclyl,” as used herein, refers to a cyclic heteroaliphatic group. A heterocyclic group refers to a non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quatemized. In certain embodiments, the term heterocylic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quatemized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroaryl,” as used herein, refer to stable aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl_{;} sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroarylamino” refers to a “substituted amino” of the (—NR^{h}₂), wherein R^{h} is, independently, a hydrogen or an optionally substituted heteroaryl group, as defined herein, and the nitrogen moiety is directly attached to the parent molecule.

The term “heteroaryloxy” refers to a “substituted hydroxyl” of the formula (—ORⁱ), wherein Rⁱ is an optionally substituted heteroaryl group, as defined herein, and the oxygen moiety is directly attached to the parent molecule.

The term “hydroxy,” or “hydroxyl,” as used herein, refers to a group of the formula (—OH). A “substituted hydroxyl” refers to a group of the formula (—ORⁱ), wherein Rⁱ can be any substitutent which results in a stable moiety (e.g., a suitable hydroxyl protecting group; aliphatic, alkyl, alkenyl, alkynyl, heteroaliphatic, heterocyclic, aryl, heteroaryl, acyl, nitro, alkylaryl, arylalkyl, and the like, each of which may or may not be further substituted).

The term “imino,” as used herein, refers to a group of the formula (—OR^{r}), wherein R^{r} corresponds to hydrogen or any substitutent as described herein, that results in the formation of a stable moiety (for example, a suitable amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted). In certain embodiments, imino refers to ═NH wherein R^{r} is hydrogen.

The term “oxo,” as used herein, refers to a group of the formula (═O).

The term “stable moiety,” as used herein, preferably refers to a moiety which possess stability sufficient to allow manufacture, and which maintains its integrity for a sufficient period of time to be useful for the purposes detailed herein.

A “suitable protecting group,” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference.

The term “thio,” or “thiol,” as used herein, refers to a group of the formula (—SH). A “substituted thiol” refers to a group of the formula (—SR^{r}), wherein R^{r} can be any substituent that results in the formation of a stable moiety (e.g., a suitable thiol protecting group; aliphatic, alkyl, alkenyl, alkynyl, heteroaliphatic, heterocyclic, aryl, heteroaryl, acyl, sulfinyl, sulfonyl, cyano, nitro, alkylaryl, arylalkyl, and the like, each of which may or may not be further substituted).

The term “thiooxo,” as used herein, refers to a group of the formula (═S).

As used herein, the term “pharmaceutically acceptable salt” refers to those salts which are, within the scope of sound medical judgment, suitable for use in contact with the tissues of humans and lower animals without undue toxicity, irritation, immunological response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66,1-19, incorporated herein by reference. Pharmaceutically acceptable salts of the compounds of this invention include those derived from suitable inorganic and organic acids and bases. Examples of to pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

The term “subject,” as used herein, refers to any animal. In certain embodiments, the subject is a mammal. In certain embodiments, the term “subject”, as used herein, refers to a human (e.g., male, female, adult, or child). The subject may be at any stage of development.

The terms “administer,” “administering,” or “administration,” as used herein refers to implanting, absorbing, ingesting, injecting, or inhaling the inventive compound.

As used herein the term “inhibit” means to reduce the activity of one or more members of the UPF0054 protein superfamily to a level or amount that is statistically significantly less than an initial level, which may be a baseline level of activity.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The terms “effective amount” and “therapeutically effective amount,” as used herein, refer to the amount or concentration of an inventive compound, that, when administered to a subject, is effective to at least partially treat a condition from which the subject is suffering.

The invention further provides methods of treating a disease using a compound of the invention. The inventive method involves the administration of a therapeutically effective amount of an inventive compound to a subject (including, but not limited to, a human or other animal) in need of it. Compounds and compositions described herein are generally useful for the inhibition of the activity of the UPF0054 protein superfamily or a mutant thereof for the treatment of bacterial infection.

The compounds and pharmaceutical compositions of the invention may be used in treating or preventing any disease or condition associated with bacterial infection. The invention further includes a method for the treatment of mammals, including humans, which are suffering from one of the above-mentioned conditions, illnesses, disorders, or diseases related to bacterial infection. The method comprises a therapeutically effective amount of one or more of the compounds according to this invention or a composition thereof which is administered to the subject in need of such treatment.

The invention further relates to the use of the inventive compounds for the production of pharmaceutical compositions which are employed for the treatment and/or prophylaxis and/or amelioration of the diseases, disorders, illnesses, and/or conditions related to the UPF0054 protein superfamily, including diseases associated with bacterial infection.

The exact amount required will vary from subject to subject, depending on the species, age, and general condition of the subject, the particular compound, its mode of administration, its mode of activity, and the like. The compounds of the invention are preferably formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular patient or organism will depend upon a variety of factors including the disorder being treated and the severity of the disorder; the activity of the specific protein employed; the specific composition employed; the age, body weight, general health, sex, and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the invention may be administered orally or parenterally at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such polyethoxylated castor oil, alcohols, oils, modified oils, glycols, polysorbates, cyclodextrins, polymers, and combinations thereof. Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as poly(lactide-co-glycolide). Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active protein may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions such as pectin-containing formulations can be used. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Additionally, the carrier for a topical formulation can be in the form of a hydroalcoholic system (e.g., liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

In still another aspect of the invention, kits are provided, containing one or more of the above-described compositions of the invention. A “kit,” as used herein, typically defines a package or an assembly including one or more of the compositions of the invention, and/or other compositions associated with the invention, for example, as previously described. Each of the compositions of the kit may be provided in liquid form (e.g., in solution), or in solid form (e.g., a dried powder). In certain cases, some of the compositions may be constitutable or otherwise processable (e.g., to an active form), to for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, modifying, assembling, storing, packaging, preparing, mixing, diluting, and/or preserving the compositions components for a particular use, for example, to a sample.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, assembly, storage, packaging, and/or preparation of the compositions and/or other compositions associated with the kit. In some cases, the instructions may also include instructions for the delivery of the compositions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

U.S. Provisional Patent Application Ser. No. 61/082,069, filed Jul. 18, 2008, entitled “Nuclease Compositions and Methods,” by Davies, et al. is incorporated herein by reference.

The following examples are intended to illustrate certain embodiments of the present invention, but do not exemplify the full scope of the invention.

### Example 1

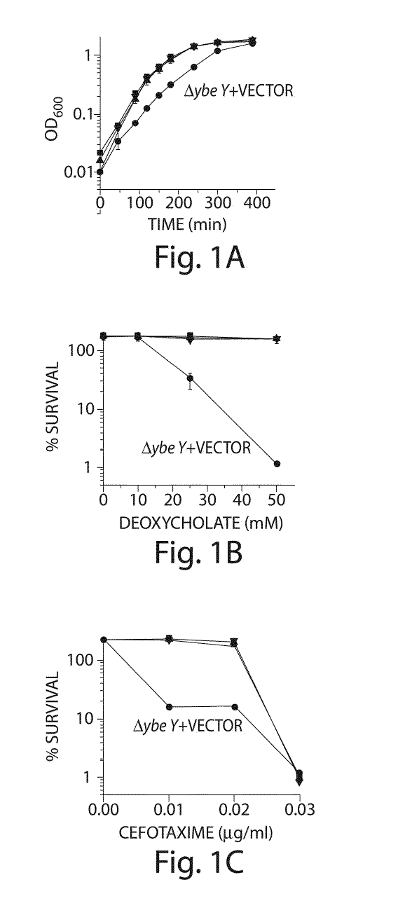

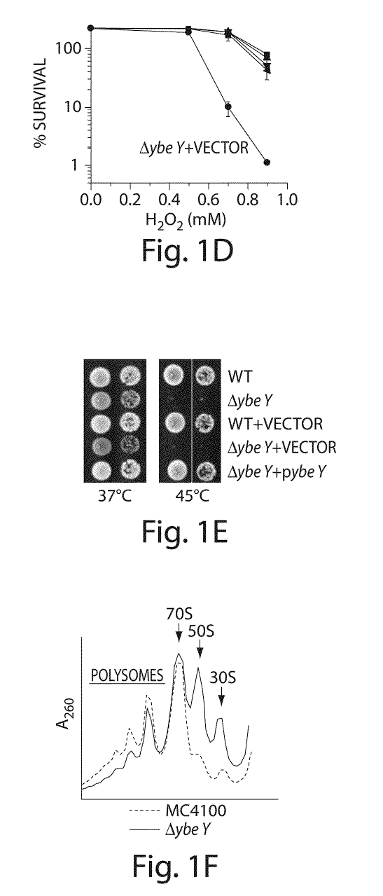

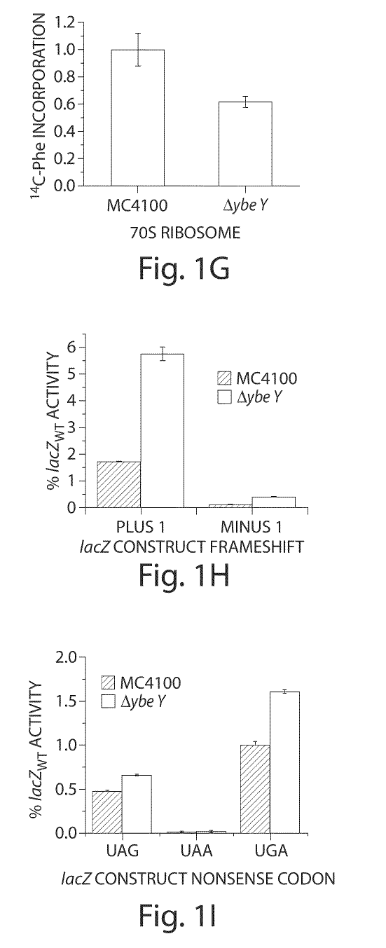

This example demonstrates that members of UPF0054 are functionally equivalent and required for stress protection. A precise deletion of the E. coli UPF0054 family member gene, ybeY, was created from the wild type strain MC4100. Deletion of ybeY in MC4100 results in an extremely pleiotropic phenotype that includes a decreased growth rate in rich medium (FIG. 1A) and significant sensitivity to numerous physiologically diverse stresses including detergents, β-lactam antibiotics, oxidative stress and temperature (FIGS. 1B-E). Deletion of ybeY from another wild type E. coli strain, MG1655, results in the same phenotypes, indicating that the effects are not strain specific. All ΔybeY mutant phenotypes were rescued by ectopic expression of ybeY (FIGS. 1A-E).

It was also demonstrated that expression of either the S. meliloti homolog SMc01113, or Bacillus subtilis homolog, yqfG, rescues the ΔybeY mutant phenotypes as effectively as ybeY itself (FIGS. 1A-D). In addition, expression of ybeY in the S. meliloti SMc01113 mutant background rescues all free-living and symbiotic phenotypes of the SMc01113 mutant (Table 2). These results demonstrate a universally conserved function for UPF0054 members in bacteria.

### Example 2

This example demonstrates that the ΔybeY mutant is defective in polysome formation. Compared to the polysome profile of the parental strain, the ΔybeY mutant has a decrease in polysomes and a striking increase in both free 50S and free 30S subunits relative to 70S ribosomes (FIG. 1F). Ectopic expression of ybeY in the ΔybeY mutant restored the normal polysome profile. Furthermore, shifting the ΔybeY mutant to 42° C. resulted in an almost complete loss of polysomes, indicating that the majority of the ΔybeY 70S ribosomes that are capable of protein synthesis have a defect that makes them temperature sensitive. Deletion of ybeY from E. coli strain MG 1655 had similar effects on its polysome profile further indicating that the effects of deleting ybeY are not strain specific.

### Example 3

This example demonstrates that the ΔybeY mutant ribosomes show decreased translational activity, decreased translational fidelity and altered translation initiation factor binding. An in vitro translation system was reconstituted with an S100 extract from the wild type strain MC4100 and 70S ribosomes from either the ΔybeY mutant or its parent MC4100. Under saturating substrate conditions, the ribosomes from the ΔybeY mutant had a significantly reduced translational activity compared to 70S ribosomes from MC4100 (FIG. 1G). This suggests that either fewer YbeY ribosomes were competent for translation or that all YbeY ribosomes have a lower translational activity.

A ribosome must not only synthesis a polypeptide but must do so accurately to produce a functional protein. The in vivo translational fidelity of the ribosomes from ΔybeY mutant was tested using lacZ constructs that contained either nonsense codons or frameshifts early in the lacZ gene. Both readthrough of nonsense codons and frameshifting showed modest increases in the ΔybeY mutant with a large effect observed for +1 frameshifts (FIGS. 1H and 1I). Thus, in addition to having a decreased translational activity, ribosomes from the ΔybeY mutant are less stringent in their decoding ability and maintenance of the reading frame. The accumulation of defects in ribosomal function, along with a defect in polysome assembly, could account for the pleiotropic nature of the ΔybeY mutant since many stress response programs rely on the upregulation of defense proteins to ward off stress or to repair damage.

The large 30S and 50S pools found in the ΔybeY mutant suggested a portion of these subunits may be defective in ribosome formation. Ribosome formation begins with the 30S subunit bound by initiation factors (IFs) 1 and 3. IF1 helps to direct the initiator tRNA (fMet-tRNA_{f}^{Met} to the ribosomal peptidyl (P) tRNA binding site and IF3 acts as an anti-30S/50S association factor. IF2, initiator tRNA and mRNA then associate with the 30S subunit. IF2 correctly positions the initiator tRNA and promotes 30S/50S association while the Shine-Dalgarno (SD) sequence of canonical mRNAs interacts with the anti-SD sequence of the 16S rRNA. IF1 and IF3 are ejected from the complex, while IF2 promotes association with the 50S subunit to the 30S complex and the resulting complex is then competent to enter the elongation phase of translation. It was found that 30S subunits and 70S ribosomes from the ΔybeY mutant showed an altered affinity for translation initiation factors that would deter 30S/50S association. Analysis of total cell lysates showed only a modest decrease in IF2 and increase in IF3 in the ΔybeY mutant (FIG. 1J). However, there was a substantial decrease in IF2 present in the ΔybeY mutant 30S fraction suggesting the bulk of the ΔybeY mutant 30S subunits are not fit for ribosome assembly. Furthermore an increase in IF3 associated with ΔybeY mutant 30S and 70S ribosomes was observed. This further supports a defect in ΔybeY mutant 30S subunits that prevents ribosome assembly and also suggests that many ribosomes that do form have a defect that promotes their disassociation. These observations suggest that a defect in the ΔybeY mutant 30S subunits may be the cause for the increased 30S and 50S pools in the ΔybeY mutant as well as for some of the translational defects.

### Example 4

This example demonstrates that the ΔybeY mutant is defective in rRNA maturation.

The results in the above Examples suggested that the polysome defect of the ΔybeY mutant resulted from a physical defect in components of the 30S and/or 50S subunits. Analysis of the protein content of the 70S, 50S, and 30S subunits by polyacrylamide gel electrophoresis did not reveal any gross differences in ribosomal-protein content.

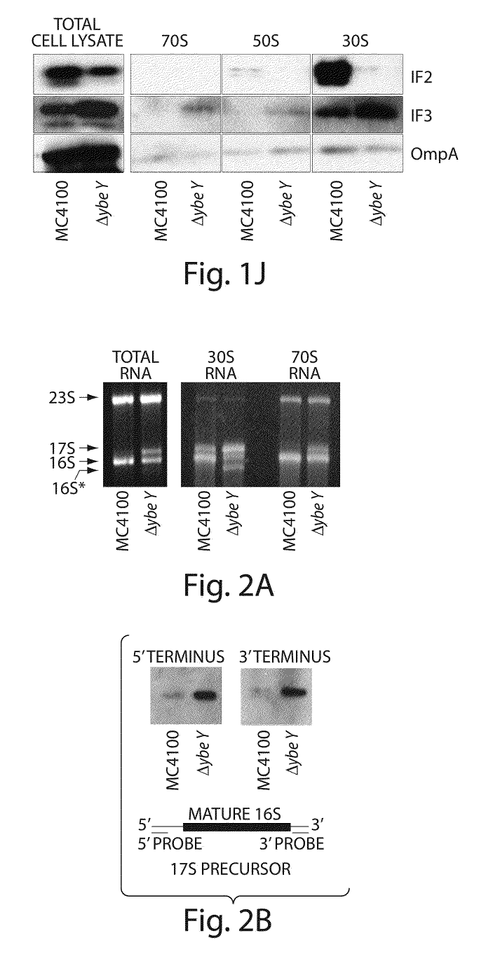

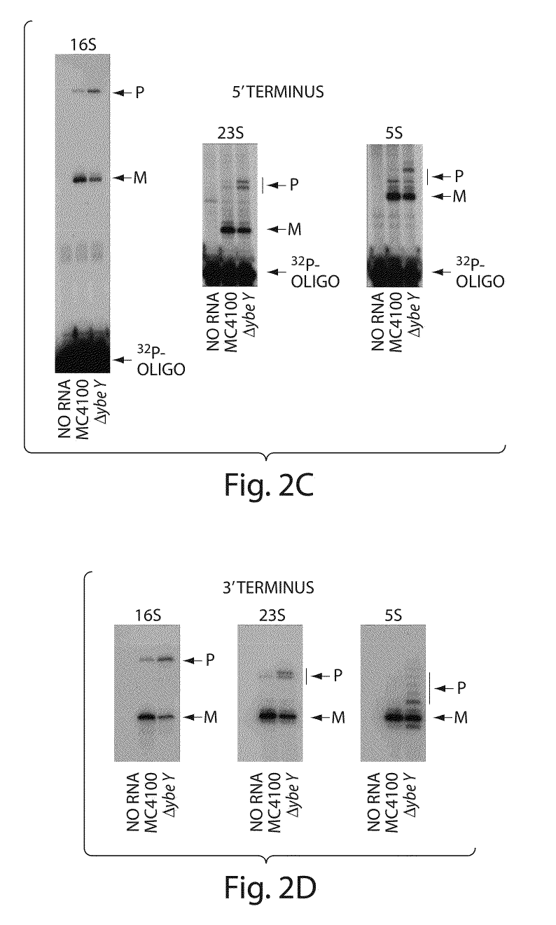

In contrast, analysis of total rRNA showed that the ΔybeY mutant accumulates a substantial amount of 17S rRNA, as well as a faster migrating species, annotated herein as 16S*, indicating a defect in 16S rRNA maturation (FIG. 2A). Ectopic expression of ybeY, the B. subtilis homolog yqfG, or the S. meliloti homolog SMc01113 in the ΔybeY mutant restores normal 16S rRNA maturation. Treatment of E. coli with a wide range of translation inhibitors including chloramphenicol, tetracycline, kanamycin, spectinomycin, or gentamycin also induce the rapid accumulation of 17S rRNA. Treatment of E. coli with either ampicillan or hydrogen peroxide does not cause the accumulation of 17S suggesting that 17S accumulation is not a general stress response but specific to translational defects.

In the wild type strain MC4100, 30S subunits contained mostly 16S rRNA along with some 17S rRNA. However, isolated MC4100 70S ribosomes contained only 16S rRNA (FIG. 2A). In contrast, the 30S subunits from the ΔybeY mutant contained much more 17S rRNA than 16S rRNA as well as a substantial amount of 16S* rRNA (FIG. 2A). The increased abundance of 17S and 16S* rRNA in the 30S subunits could account for altered translation initiation factor binding and all other effects observed above. Furthermore, in contrast to MC4100, there was also a substantial amount of 17S rRNA in ΔybeY mutant 70S ribosomes (FIG. 2A). 16S* rRNA was not found in ΔybeY 70S ribosomes suggesting it is not competent for ribosome formation and may be a non functional product of 16S rRNA misprocessing.

In agreement with the observed increase in 17S rRNA in the ΔybeY mutant, Northern blotting showed that both 5′ and 3′ termini of 17S rRNA were present at much higher levels in total rRNA extracted from the ΔybeY mutant than in total RNA from the parental strain MC4100 (FIG. 2B). primer extension and a site-specific RNase H cleavage followed by Northern hybridization method were used to map the 5′ and 3′ termini respectively of 16S rRNA (FIGS. 2C and 2D). It was found that the size of the mature and immature 16S rRNA termini in total rRNA extracted from the ΔybeY mutant and the parental strain MC4100 were identical in size and, consistent with the results above, both immature 5′ and 3′ termini of 16S rRNA were present at higher levels in total rRNA extracted from the ΔybeY mutant (FIGS. 2C and 2D). Neither 5′ or 3′ termini shorter than that of mature 16S rRNA were not observed, although, if the 5′ or 3′ terminus of the 16S* species was extensively degraded, the assays used would not have detected them. Primer extension and site-specific RNase H cleavage were used to determine the maturation state of 23S and 5S rRNA. Strikingly, along with 16S rRNA, the maturation of the 5′ and 3′ termini of both 23S and 5S rRNA were also affected with all termini showing increased amounts of the immature form in the ΔybeY mutant (FIGS. 2C and 2D). The most significant defects in rRNA termini maturation in the ΔybeY mutant appeared to be for the 5′ and 3′ termini of 16S rRNA. In sum, these results show that ybeY is critical for maturation of all rRNA species.

### Example 5

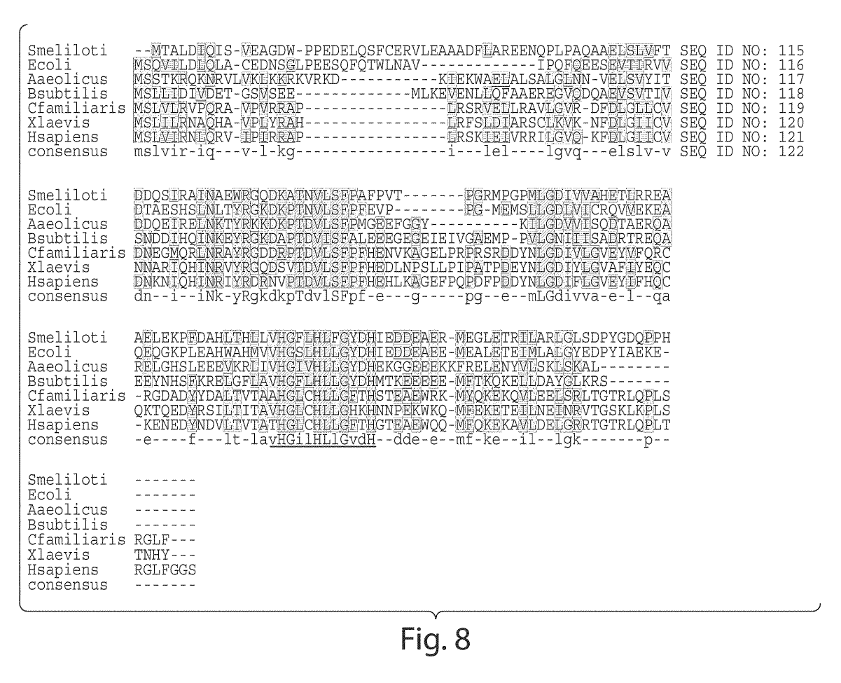

This example demonstrates that YbeY is a single stranded endoribonuclease. The primary structure of all UPF0054 family members contains a conserved H3XH5XH motif (FIG. 8). This motif led to the categorization of UPF0054 family members as putative metal-dependent hydrolases and more specifically by some as putative metal-dependent proteases due to a similar motif found in certain mammalian proteases. The structure of YbeY and two additional UPF0054 family members have recently been solved, and these structures support a putative metal-dependent hydrolytic function; however, no substrate could be found for this protein family despite extensive screening for possible substrates.

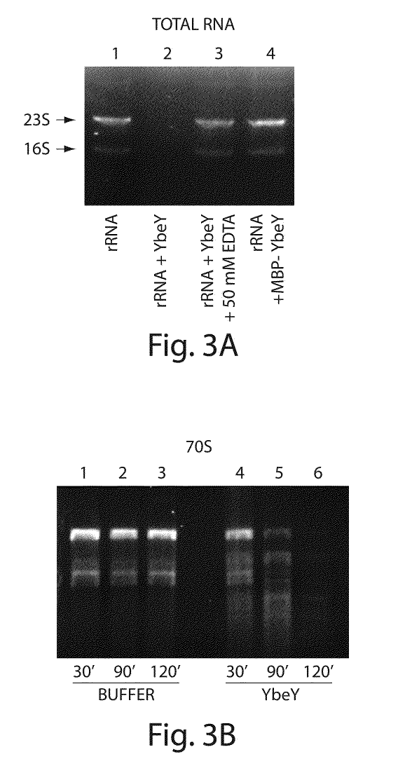

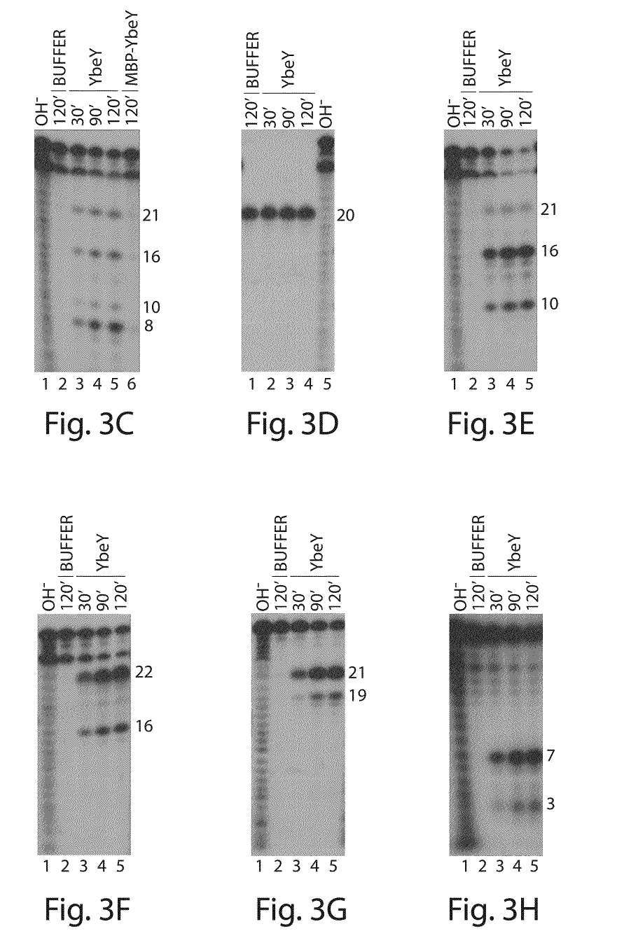

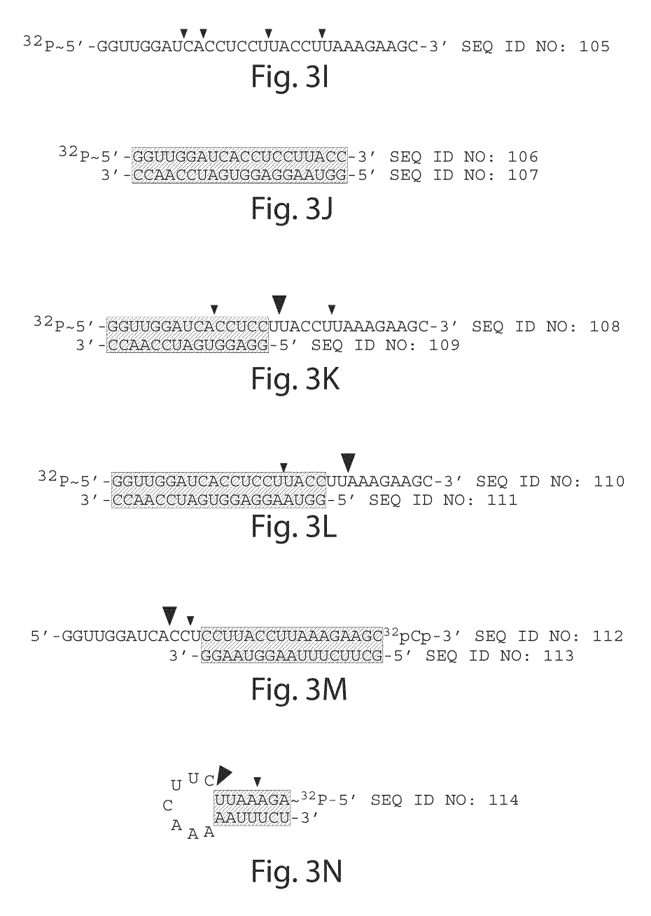

YbeY was purified as a C-terminal fusion to maltose binding protein (MBP). The MBP was then removed by TEV protease cleavage. It was found that YbeY is an RNase that can degrade total E. coli rRNA in vitro (FIG. 3A). Importantly, it was found that while YbeY was effective at degrading rRNA, the initial MBP-YbeY did not show RNase activity even at four times the molar concentration of YbeY (FIG. 3A). This indicates that the RNase activity in the YbeY preparation is indeed due to YbeY and not due to a contaminating protein. Consistent with its predicted metal-dependent hydrolase activity, the degradation of total rRNA by YbeY was inhibited by EDTA (FIG. 3A). Interestingly, YbeY's RNase activity is also inhibited by concentrations of Mg⁺² 5 mM or greater, a property that, together with its relatively low activity in vitro, may help to explain why YbeY had not been identified in previous studies of E. coli RNases. YbeY can also degrade a defined mRNA but did not degrade either double stranded or single stranded DNA.

The striking defects in rRNA maturation and ability of YbeY to cleave rRNA suggested YbeY may function directly in rRNA metabolism. In the cell, rRNA is found almost exclusively associated with ribosomal proteins as 30S subunits, 50S subunits and 70S ribosomes. To determine if YbeY could act on rRNA in the context of a ribosome YbeY was incubated with purified 70S ribosomes isolated from the ΔybeY mutant. Over time, YbeY effectively degraded the 70S ribsome producing a distinct series of bands (FIG. 3B). Primer extension and Northern blotting showed that YbeY effectively cleaved 16S rRNA from both the 5′or 3′ terminus in the context of a ribosome. These results show that YbeY can effectively remove the immature end of both the 5′ and 3′ termini and also degrade these ends past their mature termini. The in vitro inhibition of YbeY by Mg²⁺ necessitated that YbeY assays be performed in the absence of Mg²⁺. However, since ribosomes typically require at least 10 mM Mg²⁺ to maintain their structure in vitro much of the rRNA degradation by YbeY past the free immature termini may be due to a loosening of the ribosomal structure in the low Mg²⁺ buffer allowing YbeY access to portions of rRNA that would normally be inaccessible in vivo.

To explore the activity of YbeY short, sequence-defined, RNA substrates were used. Since YbeY readily cleaved rRNA, a short sequence of 16S rRNA was uses as a substrate (FIG. 3C). It was found that YbeY cleaved this single-stranded RNA (ssRNA) substrate producing a specific cleavage pattern (FIG. 3C). pCp was efficiently ligated to the 3′ termini of all YbeY cleavage products strongly suggesting that YbeY cleaves to produces a free 3′ hydroxyl terminus (data not shown). Interestingly, it was found that YbeY was unable to cleave a perfect RNA duplex (FIG. 3D) suggesting that either YbeY can only cleave ssRNA or that YbeY first needs to bind ssRNA before it can cleave its substrate. To separate these two possibilities, YbeY activity was assayed on two to partially double-stranded RNA (dsRNA) substrates each containing a ssRNA 3′ terminus (FIGS. 3E and 3F). YbeY cleaved the ssRNA 3′ termini producing a major cleavage product +1 or +2 bases from the beginning of the duplex (FIGS. 3E and 3F, respectively). YbeY was also able to produce a minor single cleavage inside the double-stranded portion of each substrate even though it was unable to do so on the same dsRNA substrate that lacked a ssRNA 3′ terminus (compare FIGS. 3D and 3F). YbeY activity on a third partially dsRNA substrate with a free 3′ ssRNA terminus that had a different nucleotide sequence was assayed. It was found that the major YbeY cut occurred flush with the dsRNA end. Without wishing to be bound by any theory, cleavage inside the duplex close to the end is believed to be due to breathing of the dsRNA terminus that allows YbeY access for cleavage. These results indicate the YbeY is predominantly a single-stranded RNase that can cleave close to dsRNA helix.

It was next asked if a similar RNase activity would be observed for YbeY if a partially duplexed RNA substrate having a 5′ ssRNA terminus was used (FIG. 3G). YbeY cleaved the ssRNA portion of this substrate and produced a major cleavage product +2 bases from the duplexed end. However, with this substrate no cleavage events were observed inside the duplex. The ability of YbeY to cleave ssRNA from either 3′ or 5′ termini as well as the discrete cleavage product pattern suggested YbeY is an endoribonuclease. To confirm this possibility YbeY activity was assayed on a hairpin substrate having a perfectly paired blunt terminus (FIG. 3H). It was found that YbeY efficiently cleaved this substrate within the loop strongly supporting an endoribonuclease activity for YbeY. The ability of YbeY to cleave within a loop structure also explains the capability of YbeY to degrade total rRNA in vitro, which has an extensive series of hairpins in its secondary structure.

### Example 6

This example demonstrates that ybeY is critical for the maturation of all rRNA species and shows a strong genetic interaction with RNase III (rnc) and RNase R (rnr). Disruption of ybeY results in a pleiotropic mutant.

To explore the cellular role of YbeY further, the rRNA profile of the ΔybeY mutant was compared to that of several well-characterized E. coli RNase mutants disrupted in genes rnc (RNase III), rnd (RNase D), rnt (RNase T), rph (RNase PH), cafA (RNase G), pnp (PNPase) or rnr (RNase R). Each of the above well-characterized RNase mutants were transduced into the ΔybeY mutant background, and the rRNA profiles of these double mutants were examined. Specific defects in the 5′ and 3′ termini of 16S, 23S and 5S rRNA were confirmed for each strain using primer extension and site-specific RNase H cleavage as described above.

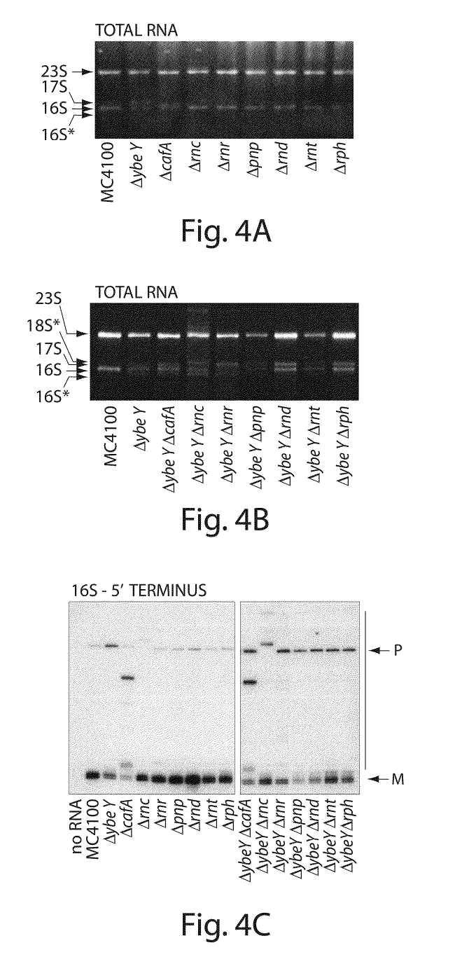

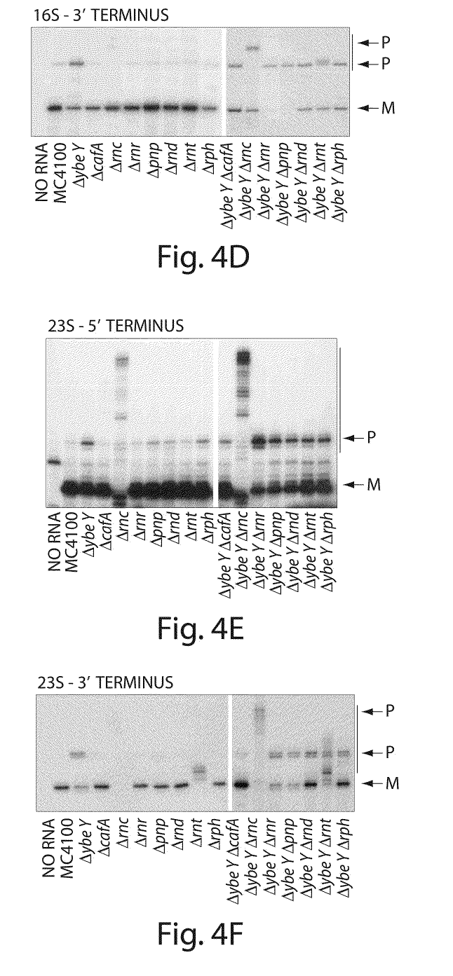

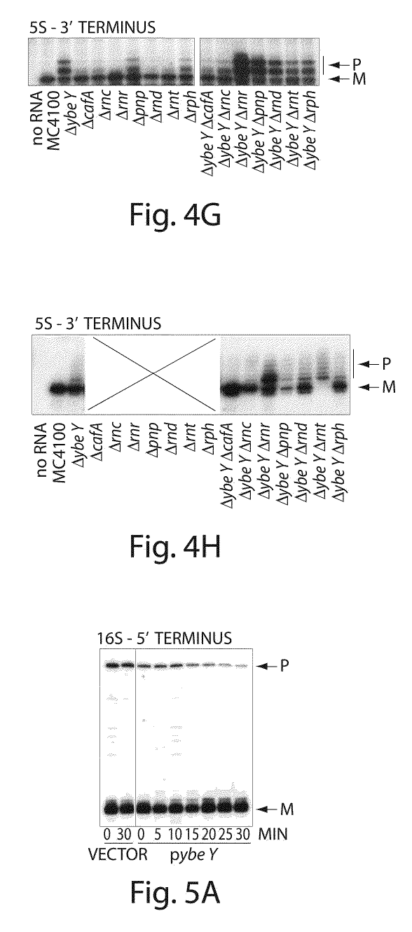

Profiles of total rRNA showed that the ΔybeY mutant had the greatest defect in 16S rRNA maturation (FIG. 4A). Primer extension and site-specific RNase H cleavage confirmed this result showing high levels of both 5′ and 3′ immature 16S rRNA termini in the ΔybeY mutant compared to the other RNase mutants (FIGS. 4C and 4D). The ΔcafA mutant also showed defects in 16S rRNA 5′ termini maturation; however, this strain still did not show the high levels of fully immature 16S rRNA 5′ termini found in the ΔybeY mutant (FIG. 4C). Only the ΔybeY mutant showed high levels of immature 16S rRNA 3′ termini accumulation.

Disruption of either rnc or rnr showed significant alterations to the gross rRNA profile of the ΔybeY mutant (FIG. 4B). It was observed the most striking defect in the ΔybeY Δrnc mutant where we no longer observed 17S rRNA but instead a new rRNA species migrating slower than 17S rRNA accumulated. Primer extension and site-specific RNase H cleavage confirmed this new precursor as a 16S rRNA derivative (FIGS. 4C and 4D). It is suggested that the new species observed in the ΔybeY Δrnc mutant is 18S rRNA and that the stabilization of 18S rRNA in the ΔybeY Δrnc mutant indicates that YbeY helps to mature 18S rRNA directly in an rnc mutant.

The ΔybeY Δrnr mutant showed a substantial decrease in 16S rRNA and reciprocal increase in both 17S and 16S* rRNA (FIG. 4B). This result was confirmed by primer extension and site-specific RNase H cleavage (FIGS. 4C and 4D). Furthermore, primer extension of 16S rRNA 5′ termini showed a dramatic increase in new partially mature and degraded 5′ termini in the ΔybeY Δrnr mutant. RNase R has been shown to function as a scavenging RNase removing non functional rRNA from the cell, so this role for RNase R is consistent with the increased amount of intermediate and degraded 16S rRNA observed by primer extension. It also suggests that loss of ybeY causes the cell to incorrectly process large amounts of 16S rRNA that RNase R would normally help degrade.

Primer extension and site-specific RNase H cleavage analysis also showed that to deletion of ΔybeY inhibited maturation of 23S and 5S rRNA with the greatest effect on 23S and 5S rRNA 5′ maturation (FIGS. 4E-4H) compared to the other RNase mutants. Loss of pnp and rph also caused accumulation of immature 5S rRNA precursor implicating these two RNases in this step of rRNA maturation as well. While disruption of RNase III significantly slows release of rRNA precursors from the original rRNA transcript, YbeY is the first RNase shown to be required for maturation of all the subsequent steps in biogenesis of 16S, 23S and 5S rRNA.

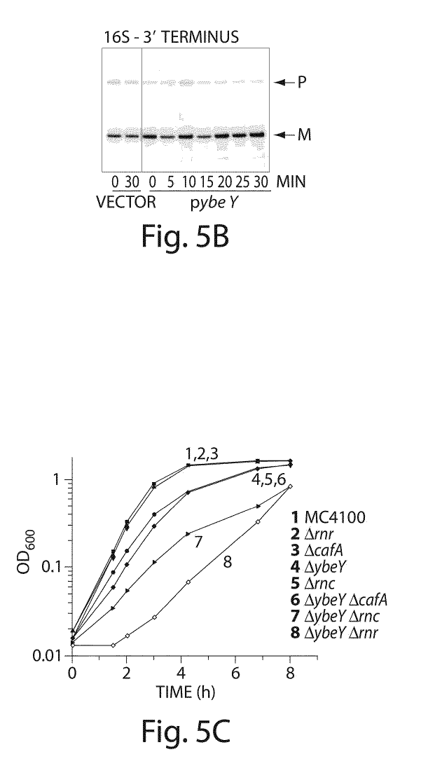

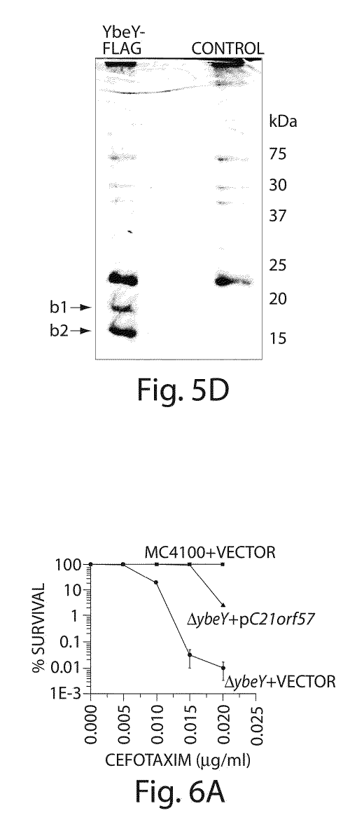

To examine the effect of YbeY on rRNA maturation in vivo, the expression of ybeY was induced from an arabinose inducible vector in the ΔybeY mutant, and the maturation state of 16S rRNA 5′ and 3′ termini was examined over time. a C-terminal FLAG tag was attached to inducible ybeY. Addition of the FLAG tag had no detectable affect on the ability of ybeY to complement the Δybe Y mutant (data not shown). Induction of YbeY-FLAG is shown in. Addition of arabinose to the growth media showed a ybeY dependent maturation of both 16S rRNA 5′ and 3′ termini over the time course (FIGS. 5A and 5B). Both termini appear to mature with similar kinetics.

It was hypothesized that RNases involved in the same pathway may be coregulated. To explore this a C-terminal FLAG tag was attached to the genomic copy of ybeY. Addition of the FLAG tag had no detectable affect on physiology (data not shown). It was determined that YbeY is present at approximately 1000±500 molecules per cell during exponential growth in rich medium at 37° C. the above set of known RNases were then disrupted, and the YbeY-FLAG expression levels were determined. Interestingly, it was found that disruption of rnc caused a small but reproducible increase in YbeY consistent with these two enzymes possibly sharing a biochemical pathway.

Further strengthening a connection between YbeY and RNase III and RNase R, the greatest physiological effect was observed in the ΔybeY Δrnc and ΔybeY Δrnr mutants. Both of these double mutants showed a substantial decrease in grow rate in rich media (FIG. 5C). In sum, these results indicated that YbeY plays a central role in rRNA processing possibly effecting rRNA maturation immediately after RNase III cleavage.

### Example 7

This example demonstrates that YbeY associates with ribosomal proteins in vivo. To further understand the in vivo role of YbeY, co-immunoprecipitation was used followed by MALDI-TOF mass spectrometry to identify interacting partner proteins. Using this approach, ribosomal proteins S7, S11 and L6 interacting with YbeY were identified (FIG. 5D). These observations suggest that YbeY interacts directly with the ribosome in vivo.

### Example 8

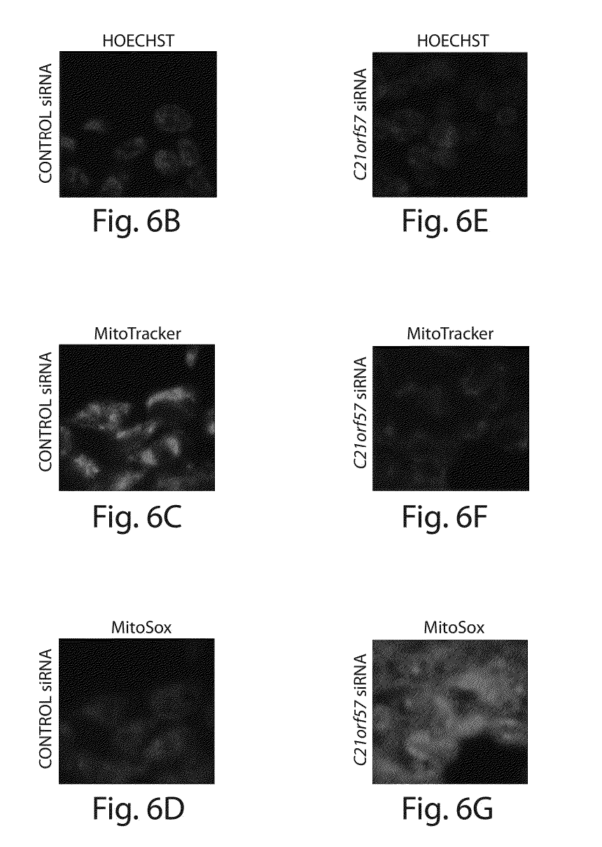

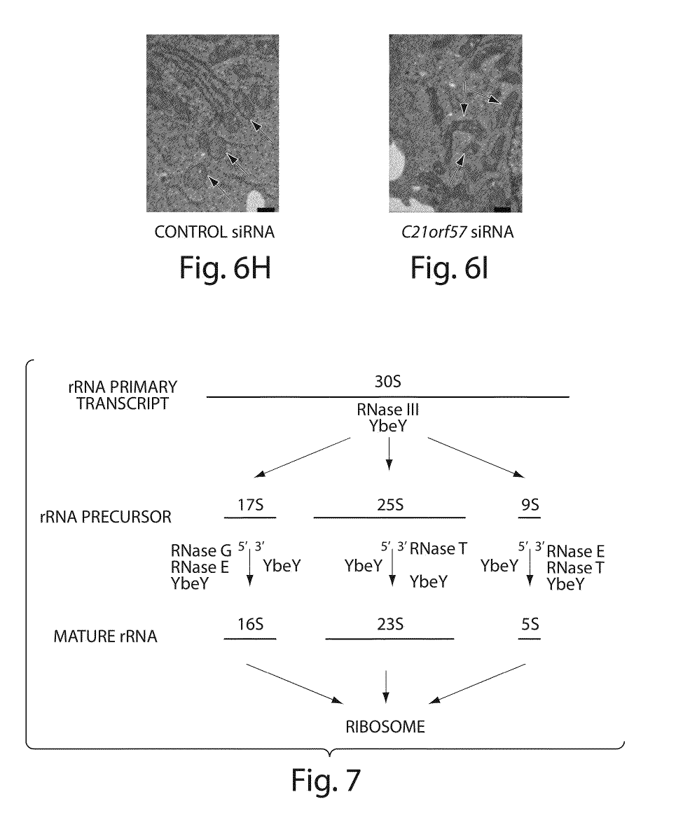

This example demonstrates characterization of the human homolog of YbeY. The human UPF0054 homolog, designated C21orf57, is encoded on the long arm of chromosome 21. Remarkably, expression of C21 orf57 in the ΔybeY mutant partially complemented the β-lactam sensitivity, temperature sensitivity, and rRNA maturation phenotypes (FIG. 6A), suggesting a shared function.

Using siRNA, a 4-fold knockdown of the C21orf57 transcript was achieved in the human cell line HEK 293T, which has been shown to express C21orf57 (www.ncbi.nlm.nih.gov/UniGene/ESTProfileViewer). It was found that these cultures showed increased mitochondrially generated superoxide at both the population and single cell level relative to the siRNA control treated culture (FIGS. 6B-6G). Furthermore, electron micrographs of cells under C21orf57 knockdown conditions revealed that the mitochondria were misshapen and stained darkly relative to control samples (FIGS. 6H and 6I), characteristics which have been associated with mitochondrial dysfunction.

### Example 9

YbeY activity was tested on a 16S rRNA 3′ terminus mimic. The mimic was a 50 nucleotide ssRNA that contained 17 nucleotides of the mature 16S rRNA 3′ terminus plus the 33 nucleotides of the immature 16S rRNA 3′ terminus (FIG. 9A). YbeY cleaved this substrate to generate a mature 3′ terminus (FIG. 10). However, YbeY also produced five additional bands suggesting that while it can generate a mature 16S rRNA 3′ terminus, YbeY may require more context than just the 16S rRNA to favor cleavage at the maturation site. For FIG. 10, immature 16S 3′ terminus mimic was incubated with and without YbeY in lanes (i), immature 16S 3′ terminus mimic in a structure resembling that when pairing with immature 16S 5′ termini was incubated with and without YbeY in lanes (ii), and immature 16S 3′ terminus mimic paired with a short mRNA resembling a structure found during protein synthesis was incubated with and without YbeY in lanes (iii). The 16S 3′ terminus mimic contains 17 bases of mature 16S 3′ termini plus the 33 bases found in the immature precursor. Schematics of the substrates are shown at the top of the figure above the lanes. “B” is buffer-only control, and 4 μg of YbeY was used in each assay.

The sequence of E. coli 16S rRNA may form a structure in which the immature 5′ and 3′ termini from 16S rRNA could pair close to the cleavage site of the mature 16S rRNA 3′ teminus. YbeY may require this paired structure to direct its activity to the 3′ maturation site. A substrate was constructed to mimic this pairing (FIG. 9A); however, when YbeY was incubated with this substrate, a similar pattern was generated as that observed for 16S rRNA 3′ terminus mimic alone (FIG. 10).

The results described herein suggested that YbeY can associate with 70S ribosomes and that maturation of the 16S rRNA 3′ terminus may occur in a structure similar to what is observed during protein translation.

In some cases, translation elongation begins with a 70S ribosome containing mRNA bound to the 3′ terminus of 16S rRNA at the anti-Shine Dalgarno sequence, an initiator tRNA at the first ATG codon, and an open A-site open for incoming tRNA. If the 16S rRNA were still immature, this would create a flap like structure (FIG. 9A). A minimal substrate was constructed to mimic this complex and was found to enhance YbeY cleavage at the maturation site (FIG. 10). This result suggested that YbeY may recognize the 16S rRNA 3′terminus in the context of active protein synthesis.

### Example 10

This example provides the methods used in Examples 1-9.

Strains, plasmids, growth conditions and DNA manipulations. Strains and plasmids are shown in Table 1. Strains were grown aerobically in Luria-Bertani (LB) at 37° C. Antibiotics for strain selection were used at the following concentrations, ampicillin 100 μg/ml, chloramphenicol 20 μg/ml, spectinomycin 100 μg/ml and kanamycin 25 μg/ml. Allele deletion was performed using the methods of Warner. Allele transfers were done by P1 transduction. DNA manipulations were performed according to the methods of Sambrook.

Phenotypic analysis. Stress and plant assays were performed as previously described.

Polysome and rRNA analysis. Polysome profiles were obtained as described previously. rRNA was extracted from logarithmically growing cultures or separated ribosome fractions using Qiagen RNeasy Mini Kit. 16S and 23 rRNA were separated using Synergel/Agarose gel electrophoresis as described previously. Primer extension assays were performed using Superscript II reverse transcriptase (Invitrogen) as per manufacturer's instructions with primers annealing to the 5′ portion of 16S, 23S, and 5S rRNA, respectively (16S_{—}5′: 5′-CGACTTGCATGTGTTAGG-3′ (SEQ ID NO: 97); 23S_{—}5′: 5′-GGGCATCCACCGTGTACGCTTAGTCG-3′ (SEQ ID NO: 98); 5S_{—}5′: 5′-GGGGTCAGGTGGGACCACCGCGCTA-3′ (SEQ ID NO: 104)). The site-specific RNase H cleavage assay was performed as described previously followed by Northern hybridization using primers specific for the mature 3′termini of 16S, 23S, and 5S, respectively. 100 ng of total MC4100 or mutant RNA were used for each assay. All reactions were separated on denaturing polyacrylamide gels.

In vitro translation and lacZ assays. In vitro translation assays were performed as described previously. ¹⁴C-Phe incorporation was determined by precipitation and scintillation counting. LacZ assays were performed as described.

Protein purification. Addition of a C-terminal FLAG tag to the genomic locus of YbeY did not have any effect on RNA maturation or MC4100 physiology (data not shown) indicating that the C-terminal FLAG tag did not affect YbeY activity. ybeY was cloned into pET28A with an N-terminal His-MBP-TEV(site) tag and a C-terminal FLAG tag. YbeY expression was induced with 1 mM IPTG in LB containing 0.2% glucose and kanamycin. Pellets were resuspended in 10 mM Tris pH 7.4, 200 mM potassium acetate, and 5 mM 2-mercaptoethanol. Cells were disrupted by sonication. Protein lysates were clarified by centrifugation, loaded onto an amylose column and washed with 10 column volumes of lysis buffer. Protein was eluted in 10 ml lysis buffer with 10 mM maltose. The MBP tag was removed using TEV protease (Promega) followed by size exclusion chromotagraphy. TEV protease alone did not show RNase activity. Fractions containing YbeY were collected and concentrated in a buffer of 10 mM Tris pH 7.4, 200 mM potassium acetate, 5 mM 2-mercaptoethanol and 10% glycerol. The YbeY preparation was then incubated with Ni-NTA resin in batch to remove residual TEV protease. The preparation was passed again over a size exclusion column. Fractions containing YbeY were concentrated in a buffer of 10 mM Tris pH 7.4, 200 mM potassium acetate, 5 mM 2-mercaptoethanol and 10% glycerol and stored at −80° C.

In vitro cleavage assays. All YbeY RNase assays were carried out in 50 mM HEPES pH 7.5 in a 20 μl final volume and used 0.2 μg of purified YbeY or 8.0 μg of MBP-YbeY. For assays using total rRNA or folA mRNA as substrate, 800 ng of total E. coli rRNA or synthesized folA mRNA was incubated with YbeY or MBP-YbeY for 30′ at 37° C., separated on an agarose/synergel mix gel and stained with ethidium bromide. Where indicated EDTA was added to 50 mM and MgSO₄ was added to 5 mM. For assays using ΔybeY mutant 70S ribosomes as substrate, 800 ng of ΔybeY mutant 70S ribosomes was incubated with YbeY for the indicated time at 37° C., separated on an agarose/synergel mix gel and stained with ethidium bromide. Short RNA substrates were labeled at their 5′ or 3′ terminus with ³²P as indicated in FIG. 3. For each reaction, 200 pmol of short RNA substrate was incubated with YbeY or MBP-YbeY for the indicated time at 37° C. Products were separated on denaturing PAGE. The ladder used for each substrate was made by alkaline hydrolysis of the respective substrate. A synthesized short rRNA of defined length was used as a reference for numerating the ladder.

Protein lysates and immunoblots. Strains were lysed by two passages through a bead beater with glass beads. Immunoblotting was performed as previously described, loading equal amounts of protein for each sample.

Co-immunoprecipitation and peptide identification. E. coli strain MC4100 expressing either native YbeY or a C-terminal FLAG-tagged YbeY were grown to mid exponential phase in LB at 37° C. Cultures were concentrated and then lysed using Bug Buster native lysis buffer (EMD Biosciences). Lystaes were clarified by centrifugation and incubated with sepharose beads conjugated with an M2 anti-FLAG antibody (Sigma) at 4° C. for 3 h. Beads were pelted and washed 3 times. Protein sample buffer was added directly to the beads, boiled, and then loaded on a 4-12% SDS PAGE gel for protein separation. Proteins were stain using Coomassie Brilliant Blue dye. Bands were excised from the gel and submitted for MALDI-TOF mass spectrometry peptide identification at the MIT biopolymers laboratory.

Transfection of mammalian cells. HEK293T cells were maintained in DMEM (4,500 mg/l glucose; Cellgro) supplemented with 10% fetal bovine serum (Atlanta Biologicals, Inc.), 2 mM glutamine, 100 units/ml of penicillin and 100 μg/ml of streptomycin (Invitrogen) at 37° C. in a 5% CO₂ atmosphere. Eighteen to twenty hours before transfection, cells were subcultured into 24-well plates. Transfection with an siRNA pool targeting C21orf57 (Dharmacon catalog #M-031405-01) was as described before with minor modifications. Briefly, cells at approx. 60 - 70% confluence were transfected with siRNA (50 nM final) using Effectene (Qiagen); 24 hours later, the siRNA transfection was repeated. 48 hours post-transfection, cells were diluted to a lower cell density, cultivated for another 14-20 hours, and then processed for cell staining and analysis of total protein synthesis.

Staining of mammalian cells. Approximately 72 hours post-transfection, cells (grown on glass cover slips) were stained with MitoSox Red (5.0 μM final), MitoTracker Green (0.5 μM final) and Hoechst 34580 (100 μg/ml final) dyes (all from Molecular Probes) for 30 min at 37° C. Cells were washed thoroughly with pre-warmed buffer, mounted and imaged. In parallel, cells from two wells were pooled, stained as described above, and the fluorescence emission spectra of MitoSox Red and MitoTracker Green were recorded by using a spectrofluorometer and Felix 1.42b software (Photon Technology International). The ratio of maximum fluorescence of MitoSox Red (λₘₐₓ=580 nm) normalized to MitoTracker Green FM (λₘₐₓ=516 nm) is given.

### Example 11

Descriptions of the figures referred to above are provided below.

FIGS. 1A-1J show phenotypic analysis of the E. coli ΔybeY mutant. (FIG. 1A shows growth curves of MC4100 (WT) and the ΔybeY mutant complemented strains in LB at 37° C. Doubling times: 40±2 min (ΔybeY mutant) vs. 28±3 min (MC4100). FIGS. 1B-1E show the sensitivity of the ΔybeY mutant to stresses (FIG. 1B) deoxycholate, (FIG. 1C) cefotaxim, (FIG. 1D) hydrogen peroxide (H₂O₂) and (FIG. 1E) temperature. The curve for the ΔybeY mutant with empty vector only (ΔybeY+vector) is shown on each plot for clarity. UPF0054 homologs: ybeY (E. coli), yqfG (B. subtilis) and SMc01113 (S. meliloti). WT+vector (▪), ΔybeY+vector (), ΔybeY+pybeY (▴), ΔybeY+pyqfG (▾) and ΔybeY+pSMc01113 [symbol] “p” indicates that the gene indicated is expressed from a plasmid. FIG. 1F shows the polysome profile for MC4100 and the ΔybeY mutant. The positions of polysomes 70S, 50S, and 30S species are indicated. (FIG. 1G shows an in vitro translation assay under saturating substrate conditions. MC4100 (WT) S100 fractions were mixed with equal amounts of MC4100 or ΔybeY mutant 70S ribosomes in an in vitro translation reaction as described in the methods sections. Translational activity is normalized to MC4100 70S ribosome reactions. Reactions proceeded for 15 min. Plasmids expressing lacZ containing (FIG. 1H) nonsense codons or (FIG. 1I) frameshift mutations (+1 or −1) were transformed into MC4100 and the ΔybeY mutant. LacZ activity was assayed as described in the methods section and normalized to the expression from a wild type lacZ gene which was set at 100%. For clarity, wild type lacZ expression has been omitted from the plots. (FIG. 1J shows immuoblots identifying IF2 and IF3 in whole cell lysates, 30S subunit, 50S subunit and 70S ribosome fraction of MC4100 and the ΔybeY mutant. Immunoblots for OmpA is used as a loading control. Equal A₂₆₀ amounts were loaded for the 30S, 50S and 70S fractions.

FIGS. 2A-2D show rRNA analysis of MC4100 and the ΔybeY mutant. (FIG. 2A) Total RNA, 30S subunit rRNA and 70S ribosome rRNA extracted from the MC4100 and the ΔybeY mutant. The positions of 16S, 17Sand 23S rRNA are indicated judged by their mobility. The accumulation of 16S rRNA precursor (17S rRNA) was not due to the slower growth rate of the ΔybeY mutant (strains were grown in minimal medium where MC4100 and the ΔybeY mutant show similar doubling times; data not shown). (FIG. 2B) Northern blot for 17S termini. Equal amounts of total RNA from MC4100 and the ΔybeY mutant were probed for 17S rRNA 5′ and 3′ termini. The locations of the probes are shown in red in the diagram above the blots. Approximate 17S rRNA maturation cleavage sites are indicated by arrows. (FIGS. 2C and 2D) Primer extension and site-specific RNase H cleavage assays to map the 5′ and 3′ termini of 16S, 23S and 5S rRNA from MC4100 and the ΔybeY mutant respectively. “P” and “M” indicate precursor and mature forms of the rRNA respectively. Total RNA was prepared from MC4100 and the ΔybeY mutant strains as described in the Methods.

FIGS. 3A-3H show YbeY RNase activity. (FIG. 3A) YbeY activity against total E. coli rRNA. The position of 16S and 23S rRNA are indicated. (FIG. 3B) A time course for YbeY activity against purified 70S ribosomes from the ΔybeY mutant. (FIGS. 3C-3H) Time course of YbeY and MBP-C21orf57 activity against short RNA substrates. A schematic and respective cleavage pattern image for each RNA substrate from FIGS. 3C-3H are shown in FIGS. 3I-3N, where FIG. 3I corresponds to FIG. 3C; FIG. 3J corresponds to FIG. 3D; FIG. 3K corresponds to FIG. 3E; FIG. 3L corresponds to FIG. 3F; FIG. 3M corresponds to FIG. 3G; and FIG. 3N corresponds to FIG. 3H. The positions of major (▾) and minor (▾) cut sites are indicated above each substrate schematic. The length of each cleavage product is indicated in nucleotides on the left side of each image. The position of each product was determined using the alkaline hydrolysis ladder (OH—) of the respective substrate and a synthesized marker.

FIGS. 4A-4H show comparison of rRNA from the ΔybeY mutant and seven well characterized E. coli RNase mutants. The relevant genotype from which the rRNA was extracted is indicated under each lane. The parental strain MC4100 rRNA is shown in each case as a control. (FIGS. 4A-4B) Total rRNA from single and double RNase mutants. The position of 23S, 18S, 17S and 16S* are indicated. (FIGS. 4C-4H) Primer extension and site-specific RNase H cleavage assays to map the 5′ and 3′ termini of 16S, 23S and 5S rRNA from single and double RNase mutants. (FIG. 4C) 16S rRNA 5′ terminus mapping. (FIG. 4D) 16S rRNA 3′ terminus mapping. (FIG. 4E) 23S rRNA 5′ terminus mapping. (FIG. 4F) 23S rRNA 3′ terminus mapping. (FIG. 4G) 5S rRNA 5′ terminus mapping. (FIG. 4H) 5S rRNA 3′ terminus mapping. “P” and “M” indicated the precursor and mature form for each rRNA.

FIGS. 5A-5D show time course monitoring the maturation of 16S rRNA 5′ and 3′ termini in the ΔybeY mutant following induction of ybeY from an arabinose inducible vector. “vector” denoted the control strain carrying an empty vector and “pybeY” denoted the strain carrying ybeY under arabinose control. “P” and “M” indicated the precursor and mature form for each rRNA. A YbeY-FLAG expression constructed was used in this assay. Induction of YbeY-FLAG with arabinose is shown in Figure S7A. (FIG. 5C) Plot showing the growth of MC4100, the ΔybeY mutant and several ΔybeY mutant double mutants in rich media at 37° C. Most ΔybeY mutant double mutants did not show a growth defect and the ΔybeY ΔcafA double mutant is shown as an example. In contrast the ΔybeY Arm and ΔybeY Δrnr mutants showed a significant decrease in growth rate. (FIG. 5D) FLAG-tagged YbeY was immunoprecipitated from an MC4100 whole cell lysate. Immunopreciptates were separated by SDS PAGE. Proteins in bands 1 and 2 were identified by MALDI-TOF mass spectrometry. Band 1 (b1) contained ribosomal proteins S7, S11 and L6. Band 2 (b2) contained subunit b of ATP synthase. An MC4100 strain carrying a non FLAG-tagged YbeY was used as the control. Size markers are shown in kD.

FIGS. 6A-6H show characterization of the human UPF0054 homolog C21orf57. (FIG. 6A) C21orf57 complements cefotaxim sensitivity of the ΔybeY mutant. MC4100+vector only (▪), ΔybeY+vector only (), ΔybeY+pC21orf57 (A). (FIGS. 6B-6G) HEK293T cells were transfected with control siRNA (FIGS. 6B-6D) or C21orf57 siRNA (FIGS. 6E-6G), stained with Hoechst dye (FIGS. 6B and 6E), MitoTracker Green (FIGS. 6C and 6F) or MitoSox Red (FIGS. 6D and 6G) and imaged using fluorescence microscopy. MitoTracker Green specifically stains mitochondria. MitoSox Red localizes to the mitochondria and reacts with superoxide to fluoresce red. (FIGS. 6H and 6I) Electron micrographs of mitochondria from HEK293T cells transfected with control siRNA (FIG. 6H) or C21orf57 siRNA (FIG. 6I). Mitochondria are indicated by black arrows.

FIG. 7 shows a genetic pathway identifying known RNases required for normal rRNA maturation. It has been demonstrated herein that ybeY is required genetically for maturation of all rRNA termini. The ΔybeY mutant accumulates significant amounts of immature 16S rRNA 3′ termini, immature 23S rRNA 5′ termini and immature 5S rRNA termini.

FIG. 8 shows sequence alignment of UPF0054 homologs from bacteria and eukaryotes. Alignments were performed using T-coffee. The red bar underlines the conserved domain that was used to classify members of this family.

TABLE 1
Bacterial strains and plasmids used in this study.

| Strain/ | | |
|---|---|---|
| plasmid | Relevant genotype and property | Source |
| Strain | | |
| MC4100 | F⁻ araD139 ΔlacU169 ΔrelA1 rpsL150 | Laboratory stock |
| | thi mot flb5301 deoC7 ptsF25 rbsR | |
| MG1655 | Wild type | Laboratory stock |
| delta-ybeY | ybeY deletion in MC4100 | This study |
| BWD10 | MC4100 carrying pBR322 | This study |
| BWD11 | delta-ybeY carrying pBR322 | This study |
| BWD12 | delta-ybeY carrying pBWD1 | This study |
| BWD13 | delta-ybeY carrying pBWD2 | This study |
| BWD14 | delta-ybeY carrying pBWD3 | This study |
| BWD15 | delta-ybeY carrying pBWD4 | This study |
| Rm1021 | SU47 Sm^{R} | |
| BWD16 | Rm1021 carrying pMSO3 | This study |
| GWBD12 | Rm1021 SMc01113::mTn5 transduced | |
| BWD17 | GWBD12 carrying pMSO3 | This study |
| BWD18 | GWBD12 carrying pBWD5 | This study |
| BL21DE3 | | Stratagene |
| CA244I⁻II⁻ | CA244, Δrnb201::tet^{R} | |
| CA244D⁻ | CA244, rnd⁻ | |
| CA244T⁻ | CA244, rnt⁻, kan^{R} | |
| CA244PH⁻ | CA244, rph⁻, kan^{R} | |
| BWD19 | MC4100 Δrnb201::tet^{R} | This study |
| BWD20 | MC4100 Δrnd::kan^{R} | This study |
| BWD21 | MC4100 rph⁻, kan^{R} | This study |
| BWD22 | MC4100 rnt⁻, kan^{R} | This study |
| BWD23 | MC4100 Δrnc::cat^{R} | This study |
| BWD24 | MC4100 ΔcafA::kan^{R} | This study |
| BWD25 | MC4100 Δpnp::kan^{R} | This study |
| BWD26 | MC4100 Δrnr::kan^{R} | This study |
| BWD27 | ΔybeYΔrnr::kan^{R} | This study |
| BWD28 | MC4100 carrying pSG25 | This study |
| BWD29 | MC4100 carrying pSG163 | This study |
| BWD30 | MC4100 carrying pSG853 | This study |
| BWD31 | MC4100 carrying pSG3/4 | This study |
| BWD32 | MC4100 carrying plac7 | This study |
| BWD33 | MC4100 carrying plac10 | This study |
| BWD34 | ΔybeY carrying pSG25 | This study |
| BWD35 | ΔybeY carrying pSG163 | This study |
| BWD36 | ΔybeY carrying pSG853 | This study |
| BWD37 | ΔybeY carrying pSG3/4 | This study |
| BWD38 | ΔybeY carrying plac7 | This study |
| BWD40 | ΔybeY carrying plac10 | This study |
| Plasmid | | |
| pBR322 | Ap^{R}, Tc^{R} | This study |
| pBWD1 | pBR322 expressing ybeY | This study |
| pBWD2 | pBR322 expressing SMc01113 | This study |
| pBWD3 | pBR322 expressing yqfG | This study |
| pBWD4 | pBR322 expressing C21orf57 | This study |
| pMS03 | Sp^{R} | |
| pBWD5 | pMSO3 carrying ybeY | This study |
| pSG25 | lacZ | |
| pSG163 | lacZ carrying UAG interruption | |
| pSG853 | lacZ carrying UAA interruption | |
| pSG3/4 | lacZ carrying UGA interruption | |
| plac7 | lacZ carrying +1 frameshift | |
| plac10 | lacZ carrying −1 frameshift | |
| pET28A | T7 promoter, MBP tag, kan^{R} | Lab stock |

TABLE 2
Complementation of the S. meliloti SMc01113::mTn5 mutant symbiotic
phenotype by ybeY. M. sativa seedling were inoculated with the indicated
strains below. After 4 weeks of growth, plant height and nodule
distribution Ire determined. The decrease in plant height and increase
in white nodules in the SMc01113::mTn5 mutant are indicative of a failed
symbiosis. “p” indicates that the gene indicated is expressed from
a plasmid. “+ vector” indicates the strain carried the vector
only as a control.

| | | White Nodules | Pink Nodules |
|---|---|---|---|
| Strain | Plant Height (cm) | (%/plant) | (%/plant) |
| Rm1021 + vector | 11.1 ± 2.2 | 1.0 ± 0.9 | 8.2 ± 2.1 |
| SMc01113::mTn5 + | 2.1 ± 1.2 | 17.8 ± 6.7 | 0 |
| EV | | | |
| SMc01113::mTn5 + | 10.5 ± 3.0 | 1.0 ± 1.3 | 8.2 ± 2.4 |
| pybeY | | | |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1-20. (canceled)

21. A composition, comprising:
an isolated UPF0054 superfamily protein contained in a solution having less than about 5 mM of Mg²⁺.

22. The composition of claim 21, wherein the isolated UPF0054 superfamily protein is YbeY.

23-37. (canceled)

38. A method, comprising:
providing a first cell and a second cell each having a growth rate in the presence of an antibiotic, the first cell being able to express a first UPF0054 superfamily protein and the second cell being able to express a second UPF0054 superfamily protein distinguishable from the first protein;
exposing the first and second cells to an agent; and
determining growth rates of the first and second cells after exposure to the agent.

39. The method of claim 38, wherein the antibiotic comprises cefotaxime.

40. The method of claim 38, wherein the antibiotic comprises kasugamycin.

41. The method of claim 38, wherein the first cell and the second cell are able to express a fluorescent protein, and wherein the growth rates are determined by determining a change in the fluorescence emitted from the first cell and the second cell as a function of time.

42. The method of claim 38, wherein the first cell and the second cell are co-cultured.

43. The method of claim 38, wherein the first UPF0054 superfamily protein is YbeY and the second UPF0054 superfamily protein is C21orf57.

44-51. (canceled)

52. A method, comprising:
administering, to a subject, an inhibitor of a member of the UPF0054 protein superfamily; and
administering an antibiotic to the subject.

53. The method of claim 52, wherein the inhibitor is an inhibitor of YbeY.

54. The method of claim 52, wherein the inhibitor inhibits UPF0054 protein activity.

55. The method of claim 52, wherein the inhibitor inhibits a UPF0054 protein-dependent cellular pathway.

56. (canceled)

57. The method of claim 52, wherein the inhibitor inhibits transcription of the UPF0054 protein.

58. The method of claim 52, wherein the inhibitor inhibits translation of the UPF0054 protein.

59. The method of claim 52, wherein the inhibitor inhibits a conformation of the UPF0054 protein.

60. The method of claim 52, wherein the inhibitor comprises a sequence that is antisense to a sequence selected from the group consisting of SEQ ID NOs: 1-95.

61. The method of claim 52, wherein the inhibitor has a molecular weight of less than about 1000 Da.

62. The method of claim 52, wherein the inhibitor is a peptide.

63. The method of claim 52, wherein the inhibitor is a polynucleotide.

64. The method of claim 52, wherein the inhibitor has a formula selected from the group consisting of:

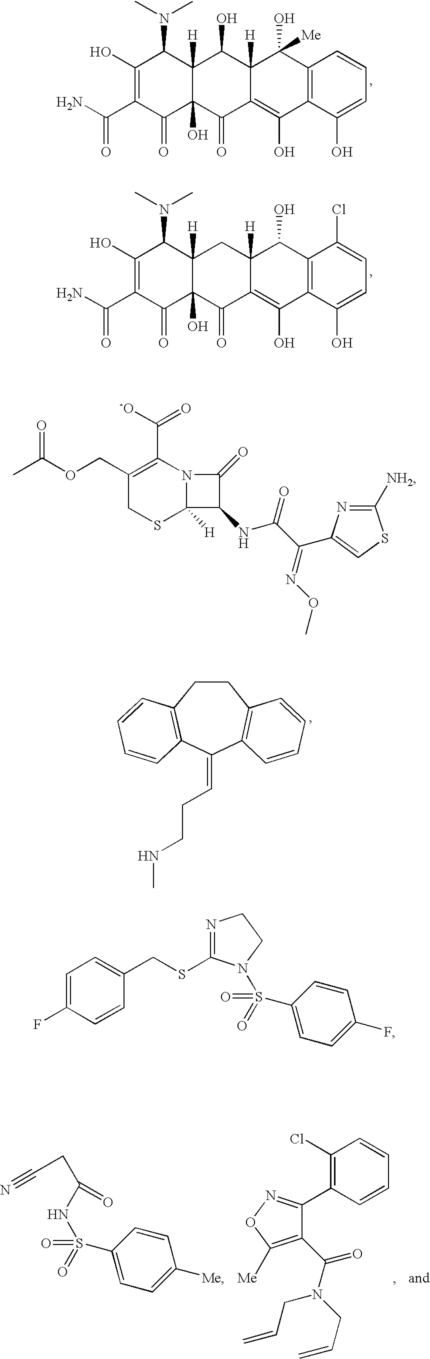

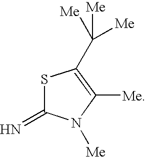

65-71. (canceled)
